(19) United States
(12) Patent Application Publication
(10) Pub. No.: US 2019/0134190 A1
(43) Pub. Date: May 9, 2019

(54) COMBINATION THERAPY WITH CPG TLR9 LIGAND

(71) Applicants: Transgene SA, Illkirch Graffenstaden (FR); Oligovax, Paris (FR)

(72) Inventors: Karola Rittner, Strasbourg (FR); Roland Kratzer, Lyon (FR); Antoine Carpentier, Paris (FR)

(73) Assignees: Transgene SA, Illkirch Graffenstaden (FR); Oligovax, Paris (FR)

(21) Appl. No.: 16/098,613

(22) PCT Filed: May 3, 2017
(86) PCT No.: PCT/EP2017/060444

§ 371 Date: Nov. 2, 2018

(30) Foreign Application Priority Data
May 4, 2016 (EP) 16305523.9

(51) Int. Cl. A61K 39/39; C12N 15/117; A61K 9/00; A61K 39/00; A61K 39/29; A61P 35/00; A61P 31/20

(52) U.S. Cl. CPC A61K 39/39; C12N 2730/10134; A61K 9/0019; A61K 39/0011; A61K 39/292; A61P 35/00; A61P 31/20; C12N 2310/315; A61K 2039/54; A61K 2039/545; A61K 2039/5256; A61K 2039/55533; A61K 2039/55561; C12N 2310/17; C12N 2320/31; C12N 2710/24143; C12N 2710/10343; C12N 15/117

(57) ABSTRACT

The present invention generally relates to an immunostimulatory combination comprising a first composition comprising a therapeutic vaccine and a second composition comprising one or more TLR9 ligand(s) such as CpG-containing oligonucleotide(s) as well as the use of such a first composition in combination with said second composition for treating a subject in need thereof. A specific embodiment is directed to the combination of a vectorized therapeutic vaccine encoding antigen(s) and a CpG-containing oligonucleotide such as Litenimod. Embodiments also include kits comprising such compositions as well as methods for treating, preventing or inhibiting diseases, in particular proliferative diseases or infectious diseases comprising administration of such first and second compositions. The invention is of very special interest in the field of immunotherapy, specifically for enhancing host's innate immune response, modifying local cytokine and chemokine profile and leukocyte populations at or around the treatment site and/or at or around the site of infection.

### TECHNICAL FIELD OF THE INVENTION

The present invention generally relates to an immunostimulatory combination comprising a first composition comprising a therapeutic vaccine and a second composition comprising one or more TLR9 ligand CpG oligonucleotide(s) as well as the use of said first composition in combination with said second composition for treating a subject in need thereof. A specific embodiment is directed to the combination of a vectorized therapeutic vaccine encoding antigen(s) and a CpG-containing oligonucleotide such as Litenimod. Embodiments also include kits comprising such compositions as well as methods for treating, preventing or inhibiting diseases, in particular, proliferative and infectious diseases comprising administration of such first and second compositions. The invention is of very special interest in the field of immunotherapy, specifically for enhancing host's innate immune response, modifying local and/or systemic cytokine and chemokine profile and leukocyte populations at or around the treatment site and/or at or around the site of infection.

### BACKGROUND

Immunotherapy seeks to boost the host's immune system to help the body to eradicate pathogens and abnormal cells. Widely used in traditional vaccination, immunotherapy is also being actively investigated as a potential modality for treating severe, chronic or life-threatening diseases in an attempt to stimulate specific and innate immune responses. A vast number of immunotherapeutics have been described in the literature for decades. In particular, several viral and non-viral vectors have now emerged, all of them having relative advantages and limits making them more appropriate to certain indications (see for example Harrop and Carroll, 2006, Front Biosci., 11, 804-817; Inchauspé et al., 2009, Int Rev Immunol 28(1): 7-19; Torresi et al., 2011, J. Hepatol. 54(6): 1273-85). For example, viral vectors such as adenovirus (Ad) (Martin et al., 2015, Gut. 64(12):1961-71) and vaccinia virus (Fournillier et al., 2007, Vaccine 25(42): 7339-53) among many others have now entered clinical development both in the cancer and infectious diseases fields. Recombinant MVA vectors generated from the attenuated non-replicative Vaccinia virus Ankara strain (MVA) are attractive candidates for their excellent safety profile and their capacity to combine robust cellular antigen-specific immune responses with a generalized stimulation of the innate immune system. TG4010 (or MVATG9931 with its research name) is a therapeutic cancer vaccine based on a modified vaccinia virus Ankara (MVA) coding for MUC1 tumor-associated antigen and human interleukin 2 (IL-2). TG4010, in combination with first-line standard of care chemotherapy in advanced metastatic non-small-cell lung cancer (NSCLC), demonstrated efficacy in two different randomized and controlled phase 2b clinical trials (Quoix et al., 2011, The Lancet Oncology 12(12): 1125-33).

However, there are limits on the immune system's ability to fight chronic diseases and cancers for several reasons. Importantly, diseased cells have evolved potent immunosuppressive mechanisms for eluding the immune system, posing a major obstacle to effective immunotherapy. Regulatory T (Treg) cell-mediated immune suppression at tumor site is now well documented (Antony and Restifo et al., 2005, J. Immunother. 28(2): 120-8; Wang et al., 2006, Cancer Res; 66(10): 4987-90). Hence, overcoming such immune blocking mechanisms may be key to successful development of more effective immunotherapeutics in cancer and infectious disease fields.

It has been suggested that suppressive Treg activity can be reversed through human Toll-like receptors (TLRs) and their ligands. Toll- like receptors (TLRs) constitute a large family of membrane-spanning receptors usually expressed in immune cells that recognize structurally conserved molecules derived from microbes once they have passed through physical barriers such as the skin or intestinal tract mucosa, and activate immune cell responses. They are believed to play a key role in the innate immune system. Most mammalian species have between ten and fifteen types of toll-like receptors that mediate host's response to different pathogens. In particular, TLR9 (Accession Number: AAF78037; Chuang, et al., 2000, Eur. Cytokine Netw. 11: 372-378) is mainly expressed by plasmacytoid dendritic cells (pDC) and B cells and recognizes specific unmethylated Cytidine-phosphate-Guanosine (CpG) motifs prevalent in microbial but not vertebrate genomic DNA (Krieg et al, 1995, Nature 374: 546-549). The biological activity of these microbial DNA elements can be mimicked by chemically synthesized oligo(deoxy)nucleotides containing such unmethylated CpG motifs (CpG-ODN) with the aim of stimulating immune effector cells.

In the infectious diseases field, in particular in the context of chronic HBV infection, it has been shown that TLR9 is important for the induction of interferons, especially interferon-a by plasmacytoid dendritic cells, and signalling through TLR9 contributes to the formation of specific structures called iMates (intrahepatic myeloid-cell aggregates for T cell population expansion) which would then favor proliferation of T cells (Huang et al., 2013, Nature Immunol, 14(6): 574-585).

Agonists of TLR9 such as CpG ODN have demonstrated potential for the treatment of cancers and infectious diseases (Hossain et al., 2015, Clinical cancer Res 21(16):3771-82; Huang et al., 2013, Nature Immunol, 14(6): 574-585). For example, Litenimod a 26 mer oligonucleotide comprising 3 CpG motifs (also called Li28 or CpG-28; developed by OligoVax, Paris, France) demonstrated in phase I, a good safety profile after intratumoral infusions in glioblastoma (GBM) patients at doses up to 20 mg (Carpentier et al., 2006, Neuro Oncol. 8: 60-66). A phase II was conducted in 31 patients with recurrent glioblastoma receiving local administration of CpG-28 into the tumor mass. Good tolerance was confirmed but a modest activity on the 6-month progression free survival (PFS) was reported although the occurrence of a few long-term surviving patients. These results could suggest a benefit of CpG-28 monotherapy for some glioblastoma patients (Carpentier et al., 2010, Neuro oncol. 12: 401-408).

Combination treatment with such TLR ligands has been explored with the goal of boosting the host's immune system and further improving vaccine efficacy (Sheiermann and Klinman, 2014, Vaccine 32(48): 6377-89; Bode et al., 2011, Expert Rev Vaccines 10(4): 499-501). Enhanced protective efficacy was reported for combinatorial approaches involving CpG ODNs and conventional preventive vaccines including Engerix (recombinantly-produced hepatitis B surface antigen); influenza Fluarix vaccine (GlaxoSmithKline Biologicals), Anthax Vaccine Adsorbed (AVA) and ISA51-adjuvanted subunit malaria vaccine (Kumar et al., 2004, Infect Immun. 72: 949-57).

A vast number of preclinical and clinical studies were conducted to evaluate the utility of adding TLR ligands to anti-tumor treatments (chemotherapy, radiotherapy, tumor antigens, monoclonal antibodies or dendritic cells, etc.). In preclinical cancer models, better survival and tumor rejection were reported for the MUC1-encoding TG4010 vector combined with a TLR3 ligand made of the double-stranded RNA from yeast viruses stabilized by the cationic lipid Lipofectin (NAB2+Lipofectin), or with the murine CpG B-type TLR9 ligand ODN1826. More specifically, the combination with locally applied NAB2+Lipofectin increased the percentage of NK cells and activated pDCs close to the tumor implantation site (Claudepierre et al., 2014, J. Virol. 88(10): 5242-55). In an orthotopic RenCa-MUC1 kidney tumor model, intravenous injection of MVA-MUC1 and the mouse-specific CpG type B TLR9 ligand ODN1826 improved the therapeutic effect of the viral vector (Fend et al., 2014, Cancer Immunol. Res. 2, 1163-74). The vaccination with TG4010 led to detectable MUC1-specific immune response and the role of the TLR9 ligand ODN1826 was the induction of a more inflammatory gene expression profile in the tumor environment. Oncolytic adenovirus engineered to express CpG ODNs were shown to increase anti-tumor effect by combining the effect of oncolysis with TLR-9-mediated CpG stimulation (Cerullo et al., 2012, Molecular Therapy 20(11): 2076-86).

However, overall, a number of factors impacted the resultant immune response and protective effects, including the specific nature of the oligonucleotide sequences (Weiner et al., 1997, Proc. Natl. Acad. Sci. USA 94: 10833-7; Hartmann et al., 2000, J. Immunol. 164: 1617-24), tumor models (Sommariva et al., 2013, doi 10.1186/1479-5876-11-25; Carpentier 2003, Frontiers in Bioscience 8, e115-127; Balsari et al., 2004, Eur. J. Cancer 40: 1275-81; Meng et al., 2005, Inst J. Cancer 116(6): 992-7), tumor burden (Weigel et al., 2003, Clin. Cancer Res. 9: 3105-14), CpG ODN formulations and administration routes (De Cesare et al., 2008, Clin Cancer Res. 14: 5512-8; EP855184).

While numerous combination therapies including CpG ODN have been proposed in the art to counteract life-threatening diseases, however their therapeutic efficacy greatly varies as discussed above. The description of prior art clearly illustrates that designing effective therapies is a difficult task due to the numerous mechanisms set up by the diseased cells and organisms to escape host's immune effector cells. Hence, there is a strong need for new combinatorial approaches permitting to improve vaccine efficacy, boost the host's immune system, in particular both specific and innate responses.

### SUMMARY OF THE INVENTION

Immunostimulatory combinations, compositions and methods disclosed herein are directed to the combined use of a therapeutic vaccine and a CpG B-type TLR9 ligand such as Litenimod 28 (also called CpG 28) to treat, prevent or inhibit a vast variety of diseases or disorders, especially those treatable by or improving with a functional immunity.

The inventors surprisingly found that administrations of a model TLR9 ligand agonist (CpG-28 also designated Li-28) in combination with a model vector (a MVA encoding a tumor-associated antigen (MUC-1) and IL-2) are surprisingly effective to reduce the volume of tumors implanted in a human cancer animal model. The combined treatment is accompanied by a significant increase of animal's survival, especially when the oligonucleotide and the viral vector are sequentially administered, with the oligonucleotide administration following the viral vector administration by 6 to 24 hours. Surprisingly, it has now been found that the combined use of the MVA vector and Li 28 generates superior immune responses characterized by a strong increase of the percentage of macrophages and activated CD69⁺ NK cells as well as by the secretion of IL-18 and IL-1 beta cytokines around the injection site. The ability of such immunostimulatory combination to provide antitumor effects together with a generalized stimulation of the innate immune system further to the antigen-specific response is a good indication that this approach could be applied to provide treatment and/or protection against a disease in a human subject, such as a proliferative disease particularly in a context of immune suppression or immunocompromised function, for example, in transplanted and cancer patients.

Accordingly, in a first aspect, the present invention relates to an immunostimulatory combination comprising at least, essentially consisting of or consisting of (a) a first composition comprising a therapeutically or an immunologically effective amount of a therapeutic vaccine and (b) a second composition comprising a therapeutically or an immunologically effective amount of an oligonucleotide having at least 21 nucleotides in length and comprising at least three hexameric motifs represented as RRCGYY (“purine-purine-C-G-pyrimidine-pyrimidine”, SEQ ID NO:13) or RYCGYY (“purine-pyrimidine-C-G- pyrimidine-pyrimidine”, SEQ ID NO:14), wherein each R occurrence is a purine nucleotide or a purine nucleotide derivative (i.e. independently A or G, wherein A is an adenosine nucleotide or an adenosine nucleotide derivative and G is a guanosine nucleotide or a guanosine nucleotide derivative); C is a cytosine nucleotide or a cytosine nucleotide derivative; G is a guanosine nucleotide or a guanosine nucleotide derivative; Y is a pyrimidine nucleotide or a pyrimidine nucleotide derivative (independently C or T wherein C is as above and T is a thymidine nucleotide or a thymidine nucleotide derivative). In one embodiment, the oligonucleotide comprises the nucleotide sequence shown in SEQ ID NO: 1 (RN₃CGYY), with N₃ being a purine (A or G) or a pyrimidine (C or T) nucleotide or a nucleotide derivative thereof, and optionally one or two additional nucleotides in 5′ (N₁N₂) and/or one or two additional nucleotides in 3′ (N₄N₅), with each of N₁, N₂, N₄, and N₅ being independently a purine (A or G) or a pyrimidine (C or T) nucleotide or a nucleotide derivative thereof. In this case, the oligonucleotide comprises one of the nucleotide sequences shown in:
SEQ ID NO: 1 (RN₃CGYY), with N₃ being a purine (A or G) or a pyrimidine (C or T) nucleotide or a nucleotide derivative thereof,
SEQ ID NO:2 (N₂RN₃CGYY), with each of N₂ and N₃ being independently a purine (A or G) or a pyrimidine (C or T) nucleotide or a nucleotide derivative thereof,
SEQ ID NO:3 (N₁N₂RN₃CGYY), with each of N₁, N₂ and N₃ being independently a purine (A or G) or a pyrimidine (C or T) nucleotide or a nucleotide derivative thereof,
SEQ ID NO:4 (RN₃CGYYN₄), with each of N₃ and N₄ being independently a purine (A or G) or a pyrimidine (C or T) nucleotide or a nucleotide derivative thereof,
SEQ ID NO:5 (RN₃CGYYN₄N₅), with each of N₃, N₄ and N₅ being independently a purine (A or G) or a pyrimidine (C or T) nucleotide or a nucleotide derivative thereof,
SEQ ID NO:6 (N₂RN₃CGYYN₄), with each of N₂, N₃ and N₄ being independently a purine (A or G) or a pyrimidine (C or T) nucleotide or a nucleotide derivative thereof,
SEQ ID NO:7 (N₂RN₃CGYYN₄N₅), with each of N₂, N₃, N₄ and N₅ being independently a purine (A or G) or a pyrimidine (C or T) nucleotide or a nucleotide derivative thereof,
SEQ ID NO:8 (N₁N₂RN₃CGYYN₄), with each of N₁, N₂, N₃ and N₄ being independently a purine (A or G) or a pyrimidine (C or T) nucleotide or a nucleotide derivative thereof, and
SEQ ID NO:9 (N₁N₂RN₃CGYYN₄N₅), with each of N₁, N₂, N₃, N₄ and N₅ being independently a purine (A or G) or a pyrimidine (C or T) nucleotide or a nucleotide derivative thereof.

In a second aspect, the present invention provides a first composition comprising a therapeutically or an immunologically effective amount of a therapeutic vaccine for use in the treatment of a disease in combination with a second composition comprising a therapeutically or an immunologically effective amount of an oligonucleotide; wherein said oligonucleotide has at least 21 nucleotides in length and comprises at least three hexameric motifs represented as RRCGYY (SEQ ID NO: 13) or RYCGYY (SEQ ID NO: 14) wherein each occurrence is as defined above.

Further aspects relate to a method for treating or preventing a disease and a method for inducing or stimulating an immune response comprising administering to a subject a combination of therapeutically effective amounts of (a) and (b) as described herein. Said induction or stimulation of the immune response is notably correlated by at least one the following properties e.g. an increased in the number of macrophages and/or an increase in the number of activated CD69+ NK cells and/or an increase in the number of KLRG1+ CD3+ CD8+ lymphocytes and/or an increase of the concentration of IL-18 and/or an increase of the concentration of IL-1 beta and/or a decrease of CD163 marker at the surface of human macrophages which is indicative of a differentiation towards M1 instead of M2 phenotype.

In one embodiment, the at least 3 hexameric motifs represented as RRCGYY (SEQ ID NO: 13) are preferably AACGTT (SEQ ID NO: 15) and those represented as RYCGYY (SEQ ID NO: 14) are preferably GTCGTT (SEQ ID NO: 16). In a more preferred embodiment, the CpG oligonucleotide comprises a nucleotide sequence as shown in SEQ ID NO: 10 (5′-TAAACGTTATAACGTTATGACGTCAT-3′) or a nucleotide sequence as shown in SEQ ID NO: 11 (5′-TCGTCGTTTTGTCGTTTTGTCGTT-3′).

In one embodiment, the therapeutic vaccine is a plasmid or a viral vector and desirably a recombinant viral vector encoding one or more polypeptide(s) of therapeutic interest selected from the group consisting of a suicide gene product, a cytokine and an antigenic polypeptide. In a preferred embodiment, the therapeutic vaccine is a replication-defective viral vector encoding an antigen with a preference for a MVA vector encoding a tumor-associated antigen. In another preferred embodiment, the therapeutic vaccine is a replication-defective adenoviral vector encoding an antigen with a preference for an adenoviral vector encoding one or more HBV antigen(s).

In one embodiment, the therapeutic vaccine and the CpG ODN are delivered to the subject sequentially with a preference for a sequential administration starting with the therapeutic vaccine followed by the CpG ODN at least at 1 hour interval. Several cycles can be envisaged.

### DESCRIPTION OF THE DRAWINGS

FIG. 1 illustrates the beneficial effect of sequential administration schedule of MVATG9931 and the CpG type B TLR9 ligand Li28 in the prophylactic RMA-MUC1 tumor model: MVATG9931 was injected sc three times (D1, 7 and 14) at the suboptimal dose of 1×10³ pfu. Ten μg of Li28 was injected sc at the same time (Oh) as MVATG9931, or 6 h or 24 h later. MUC1⁺ RMA-MUC1 tumor cells were implanted day 21 at the same flank (ipsilateral). Twelve mice per group were injected.

FIG. 2 illustrates the survival (A) and tumor rejection (B) in the prophylactic RMA-MUC1 tumor model upon the combined use of MVATG9931 and the Li28 compared to monotherapy (same experimental protocol as above except the variation of time interval between the MVA vector and Li28 injections). Li28 was injected with a delay of 24 h or 48 h in the same flank and site as the MVA vector. Controls with the empty control vector MVATGN33.1 MVA vector in monotherapy and in combination with Li28 (24 h and 48 h) were also included as well as treatment with buffer (negative control).

FIG. 3 illustrates the effect of tumor implantation either contralateral (A) or ipsilateral (B) to the MVA and/or Li28 injection sites. MVATG9931 was injected three times (D1, 7 and 14) at the suboptimal dose of 1×10³ pfu. Ten μg of Li28 was injected sc at the same site 24 h after (+24 h) or before (−24 h) MVATG9931, and either contralateral (contra) or ipsilateral (ipsi) to the MVATG9931 injection site. MUC1⁺RMA-MUC1 tumor cells were implanted day 21 in the opposed “contralateral” flank (A) or at the same “ipsilateral” flank (B).

FIG. 4 illustrates the effect of the number of injection cycles of MVATG9931 with and without Li28 in the prophylactic RMA-MUC1 tumor model. FIG. 4A: one injection cycle with MVATG9931 at 1×10³ pfu (DO) and Li28 (D1) was compared to three injection cycles of MVATG9931 (D0-D7-D14) with Li28 (D1-D8-D15) or without. FIG. 4B: two injection cycles with both components (MVATG9931 D0-D7+Li28 D1-D8) were compared to three injection cycles of MVATG9931 (D0-D7-D14) with Li28 (D1-D8-D15) or without.

FIG. 5: Analysis of cell populations around the MVA injection site: 5×10⁵ pfu of MVATG9931 were s.c. injected once or twice (D1 and D7). Twenty-four hours after the last injection (D2 or D8), mice were sacrificed, shaved skin samples comprising the injection sites were cut out and mechanically dissociated. Two skin samples per mouse from five to eight mice per group were pooled. A) provides the percentage of CD45⁺ leukocytes in the skin after one or two injection cycles (N=18). B) shows the fold induction of percentages of various cell populations after one or two injections of MVA compared to buffer-injected control groups (N=2). Cell suspensions were stained for flow cytometry analysis: pDCs were identified as a Ly6C⁺mPDCA-1⁺CD45R⁺CD11b⁻ subpopulation within living CD45⁺CD3⁻CD19⁻NKp46⁻ cells. Within the same sub-population, CD11c⁻CD11b⁺ cells were identified as Ly6G⁻Ly6C⁺F4/80⁺macrophages or Ly6G⁺Ly6C⁺7/4⁺ neutrophils. Within the CD45⁺CD3⁻ CD19⁻NKp46⁻ population, CD11c⁺ cells were divided in cDCs (CD11b⁺) and dermal DCs (Langerin⁻). Within the CD45⁺CD11c⁻CD11b⁻ cell population, NK cells were identified as CD3⁻ and NKp46⁺, and B lymphocytes as CD3⁻ and CD19⁺ cells; CD8⁺ and CD4⁺ T lymphocytes were identified within the CD19⁻ CD3⁺ cell population. The percentage of these various cell types within the total cell population was calculated, and the results were expressed as the fold induction on the basis of the values obtained with the buffer-injected control group.

FIG. 6: analysis of the cell populations around the injection site (skin) and in the draining lymph nodes (DLN) after two treatment cycles with MVATG9931 (5×10⁵ pfu) and Li28 (10 μg): A) macrophages, (N=4, 5 mice per group); B) activated CD69⁺ NK cells (N=3, 5 mice per group); C) activated CD86⁺ cDCs and dermal DCs (N=3, 5 mice per group); D) DLN absolute number of CD86⁺ cDCs and a population of CD86⁺ CD8⁻ DC (N=3, 5 mice per group). E) Lymphocytes extracted from the 10 vaccination site were tested for CD8, CD3, KLRG1 and CD127 expression. Two experiments are shown.

FIG. 7: Local cytokine/chemokine profile after two cycles of combination treatment with MVATG9931 and Li28 in C57BL/6 mice (5 mice /group). Skin samples were taken 16 hours after the last injection and cytokine expression was performed by multiplex analysis; respectively A) IL-18, B) IL-1beta, C) IL-4, D) IL-5 and E) IL-13.

FIG. 8: Effect of depletion of macrophages by Clodronate liposomes around the injection site in a tumor control experiment: Injection of 1×10³ pfu of MVATG9931 day 1 and 6, followed by 10 μg Li28 in the morning of day 2 and 7, followed by injection of 60 μl Clodronate liposomes or control liposomes in the evening of day 2 and 7. Survival rates obtained were followed in each group.

FIG. 9: Infection of murine bone marrow derived macrophages (m-CSF). C57BL/6 mice were sacrificed, bone marrow cells were isolated and differentiated to murine bone marrow derived macrophages during 8 days in the presence of m-CSF (100 μg/ml) in RPMI 10% FBS. 5×10⁵ murine macrophages were plated in 500 μl RPMI in 24 well plates and infected with either a MVA vector expressing GFP (MVA-GFP) or with a TK- and RR-oncolytic Vaccinia virus of Western Reserve strain expressing GFP (WR-GFP) at MOI of 0.1, 0.3 or 1. Two hours later, 10 μg Li28 was added and the percentage of GFP-positive cells was determined (N=2). As an alternative immune-modulator, NAB2+Lipofectin was tested.

FIG. 10: comparison of combinatorial treatment of MVATG9931 with various CpG oligonucleotides in the prophylactic RMA-MUC1 tumor model. MVATG9931 was injected three times sc (D1, 7 and 14) at the suboptimal dose of 1×10³ pfu. Ten μg of Li28, ODN2336 (human type A CpG), ODN2006 (human type B CpG), ODN2395 (human/murine type C CpG), ODN1585 (murine type A CpG) or ODN1826 (murine type B CpG) (all obtained from Invitrogen) were injected sc at the same site as MVATG9931 24 h later. MUC1⁺ RMA-MUC1 tumor cells were implanted day 21 at the same flank. Thirteen mice per group were injected.

FIG. 11: Evolution of HBsAg levels depending on time expressed (A) in ng/mL or (B) as delta log compared to baseline in different groups of AAV-HBV transduced mice (median values).

FIG. 12: Detection of IFNγ producing cells by IFNγ Elispot assay in presence of medium alone (negative control) of an Adenovirus-specific peptide (FAL) and of an HBV polymerase-specific peptide VSA. Individual mice are represented as well as mean value for each group.

### DETAILED DESCRIPTION OF THE INVENTION

### Definitions

Unless defined otherwise, all technical and scientific terms used herein have the same meaning as commonly understood by one of ordinary skill in the art to which the invention pertains.

The term “a” and “an” refers to “one” or to “more than one” of the grammatical object of the article (i.e., at least one including 2, 3, 4, 5, etc.) unless the context clearly dictates otherwise. By way of example, the term “a therapeutic vaccine” includes one therapeutic vaccine or a plurality of therapeutic vaccines, including mixtures thereof.

The term “and/or” wherever used herein includes the meaning of “and”, “or” and “all or any other combination of the elements connected by said term”.

The term “about” or “approximately” as used herein means that the exact value or range is not critical and can vary within 10%, preferably within 8%, and more preferably within 5% of the given value or range.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a composition “comprises” the recited components when such components might be part of the final composition. “Consisting essentially of” means excluding other components or steps of any essential significance. Thus, a composition consisting essentially of the recited components would not exclude trace contaminants and pharmaceutically acceptable carriers. “Consisting of” means excluding more than trace elements of other components or steps.

The terms “polypeptide”, “peptide” and “protein” are used interchangeably to refer to polymers of amino acid residues comprising at least nine amino acids covalently linked by peptide bonds. The polymer can be linear, branched or cyclic and may comprise naturally occurring and/or amino acid analogs and it may be interrupted by non-amino acids. No limitation is placed on the maximum number of amino acids comprised in a polypeptide. As a general indication, the term refers to both short polymers (typically designated in the art as peptide) and to longer polymers (typically designated in the art as polypeptide or protein). This term encompasses native polypeptides, modified polypeptides (also designated derivatives, analogs, variants or mutants), polypeptide fragments, polypeptide multimers (e.g. dimers), recombinant polypeptides, fusion polypeptides among others.

Within the context of the present invention, the terms “nucleic acid”, “nucleic acid molecule”, “polynucleotide”, “nucleic acid sequence”, and “nucleotide sequence” are used interchangeably and define a polymer of at least 5 nucleotide residues (also called “nucleotides”) in either deoxyribonucleic acid (DNA) or ribonucleic acid (RNA) or mixed polyribo-polydeoxyribonucleotides.

These terms encompass single or double-stranded, linear or circular, natural or synthetic, unmodified or modified versions thereof (e.g. genetically modified polynucleotides; optimized polynucleotides), sense or antisense polynucleotides, chimeric mixture (e.g. RNA-DNA hybrids). Moreover, a polynucleotide may comprise non-naturally occurring nucleotides and may be interrupted by non-nucleotide components. Exemplary DNA nucleic acids include without limitations, complementary DNA (cDNA), genomic DNA, plasmid DNA, DNA vector, viral DNA (e.g. viral genomes, viral vectors), oligonucleotides, probes, primers, satellite DNA, microsatellite DNA, coding DNA, non-coding DNA, antisense DNA, and any mixture thereof. Exemplary RNA nucleic acids include, without limitations, messenger RNA (mRNA), precursor messenger RNA (pre-mRNA), small interfering RNA (siRNA), short hairpin RNA (shRNA), microRNA (miRNA), RNA vector, viral RNA, guide RNA (gRNA), antisense RNA, coding RNA, non-coding RNA, antisense RNA, satellite RNA, small cytoplasmic RNA, small nuclear RNA. Polynucleotides described herein may be synthesized by standard methods known in the art, e.g., by use of an automated DNA synthesizer (such as those that are commercially available from Biosearch, Applied Biosystems, etc.) or obtained from a naturally occurring source (e.g. a genome, cDNA, etc.) or an artificial source (such as a commercially available library, a plasmid, etc.) using molecular biology techniques well known in the art (e.g. cloning, PCR, etc.).

The term “oligonucleotide” as used herein refers to a polynucleotide (RNA or DNA) subset comprising no more than 200 nucleotide units. In a preferred embodiment, the “oligonucleotide” is an oligodeoxynucleotide. In the context of the present invention, each nucleotide unit can independently contain chemical modifications and substitutions as compared to a wild-type nucleotide. The oligonucleotide can be modified at the base moiety, sugar moiety, or phosphate backbone, for example, to improve its stability, its biological half-life, its affinity its hybridization parameters, and/or its production, etc. A modified base is a base that is not guanine, cytosine, adenine, thymine or uracil. Exemplary modified bases include for example fluoro, bromo, thio acetyl, methyl, dimethyl derivatives. A modified sugar is any sugar that is not ribose or 2′ deoxyribose. Exemplary backbone modifications include for example phosphodiester, phosphorothioate, phosphorodithioate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleotide linkages as well as phosphodiester-phosphorothioate mixed backbone. Examples of chemical modifications are known to the person skilled in the art (e.g. Uhlmann et al., 1990, Chem. Rev. 90: 543); in “Protocols for Oligonucleotides and Analogs; Synthesis and Properties”, 1993, Ed S. Agrawal, Humana Press, Totowa, N.J.); and Crooke et al., 1996, Ann. Rev. Pharm. Tox. 36: 107-129). For illustrative purposes, phosphorothioate oligonucleotides may be synthesized by the method of Stein et al. (1988, Nucl. Acids Res., 16, 3209) or Crooke (1991, Anti-Cancer Drug Design 6: 609-46). Optionally, the oligonucleotide can be conjugated to a non-nucleotide compound (e.g. a functional group or a labeling compound). Various sites of conjugation are possible such as the heterocyclic base, the sugar or the phosphate linkage.

In the context of the present invention, nucleic base components or their respective abbreviated designations can be used to specify nucleotide sequences. According to the context, “A” may refer to adenine, “C” refers to cytosine, “G” refers to guanine, “T” refers to thymine and “U” refers to uracil. As used herein, the term “pyrimidine” refers to a nucleoside or nucleotide having a base component selected from the group consisting of cytosine (C) or thymine (T) or Uracil (U) whereas, the term “purine” refers to a nucleoside or nucleotide having a base component which is adenine (A) or guanine (G).

The term “CpG” as used herein refers to a dinucleotide comprising a cytosine or a cytosine analog and a guanine or a guanine analog. The oligonucleotide in use herein is characterized by comprising at least three of such CpG dinucleotides in a particular sequence context.

The term “5′” as used herein, generally refers to a region or position in a polynucleotide or oligonucleotide upstream (5′) from another region or position in the same polynucleotide or oligonucleotide.

The term “3′” as used herein generally refers to a region or position in a polynucleotide or oligonucleotide downstream (3′) from another region or position in the same polynucleotide or oligonucleotide.

The term “analog”, “mutant”, “derivative” or “variant” can be used interchangeably to generally refer to a component (polypeptide, polynucleotide, oligonucleotide, nucleoside, nucleotide, vector, etc.) exhibiting one or more modification(s) with respect to a reference component (e.g. the wild-type component as found in nature). A nucleotide or nucleoside analog can have a modified base and/or a modified sugar and/or a modified linkage. With respect to polypeptide and polynucleotide, any modification(s) can be envisaged, including substitution, insertion and/or deletion of one or more nucleotide/amino acid residue(s). When several mutations are contemplated, they can concern consecutive residues and/or non-consecutive residues. Mutation(s) can be generated by a number of ways known to those skilled in the art, such as site-directed mutagenesis, PCR mutagenesis, DNA shuffling and chemical synthetic techniques (e.g. resulting in a synthetic nucleic acid molecule). Preferred are analogs that retain a degree of sequence identity of at least 80% with the reference component. For illustrative purposes, “at least 80% identity” means 80%, 81%, 82%, 83%, 84%, 85%, 86%, 87%, 88%, 89%, 90%, 91%, 92%, 93%, 94%, 95%, 96%, 97%, 98% or 99%. In certain embodiment, at least 80% identity also encompasses 100% identity.

In a general manner, the term “identity” refers to an amino acid to amino acid or nucleotide to nucleotide correspondence between two polypeptide or nucleic acid sequences. The percentage of identity between two sequences is a function of the number of matching (e.g. identical) positions shared by the sequences, taking into account the number of gaps which need to be introduced for optimal alignment and the length of each gap. Various computer programs and mathematical algorithms are available in the art to determine the percentage of identity between amino acid sequences, such as for example the Blast program available at NCBI or ALIGN in Atlas of Protein Sequence and Structure (Dayhoffed, 1981, Suppl., 3: 482-9). Programs for determining identity between nucleotide sequences are also available in specialized data base (e.g. Genbank, the Wisconsin Sequence Analysis Package, BESTFIT, FASTA and GAP programs).

As used herein, the term “isolated” refers to a component (e.g. a polypeptide, polynucleotide, vector, etc.), that is removed from its natural environment (i.e. separated from at least one other component(s) with which it is naturally associated or found in nature). An isolated component refers to a component that is maintained in a heterologous context or purified (partially or substantially). For example, a nucleic acid molecule is isolated when it is separated of sequences normally associated with it in nature (e.g. dissociated from a chromosome or a genome) but it can be associated with heterologous sequences (e.g. within a recombinant vector). A synthetic component is isolated by nature.

The term “obtained from”, “originating from” or “derived from” is used to identify the original source of a component but is not meant to limit the method by which the component is made which can be, for example, by chemical synthesis or recombinant means.

The term “subject” generally refers to a living organism for whom any product and method of the invention is needed or may be beneficial. In the context of the invention, the subject is preferably a mammal, particularly a mammal selected from the group consisting of domestic animals, farm animals, sport animals, and primates. Preferably, the subject is a human who have been diagnosed as being or at risk of having a pathological condition such as a proliferative disease (e.g. cancer) or an infectious disease (e.g. a chronic B hepatitis caused by an HBV infection). The terms “subject” and “patients” may be used interchangeably when referring to a human organism and encompasses male and female as well as newborn, infant, young adult, adult and elderly.

As used herein, the term “host cell” should be understood broadly without any limitation concerning particular organization in tissue, organ, or isolated cells. Such cells may be of a unique type of cells or a group of different types of cells such as cultured cell lines, primary cells and dividing cells. In the context of the invention, the term “host cells” include prokaryotic cells, lower eukaryotic cells such as yeast, and other eukaryotic cells such as insect cells, plant and mammalian (e.g. human or non-human) cells as well as producer cells capable of producing the plasmid or virus-based therapeutic vaccine. This term also includes cells which can be or has been the recipient of the immunostimulatory combination described herein as well as progeny of such cells.

“Immunostimulatory combination” as used herein refers to the ability of the combined entities to enhance or potentiate the immune activity of an antigen and/or the immune protective effect in a subject exposed to the combined entities—whether specific or non-specific; humoral or cellular. Typically, the immune response observed with the immunostimulatory combination is greater or intensified in any way (duration, magnitude, intensity, etc.) when compared to the same immune response measured with each entity alone under the same conditions.

The term “ligand” generally refers to a substance that binds to a receptor of a cell and induces a biological signal.

“Treatment” as used herein refers to prophylaxis and/or therapy.

The term “therapeutic vaccine” as used herein refers to any component or group of components which is expected to cause a biological response when delivered appropriately to a subject through the presence or expression of one or more biological substance(s) (e.g. a polypeptide such as an antigen, an enzyme, a cytokine, a SiRNA, etc.).

A “therapeutically effective amount” corresponds to the amount of each active entity that is sufficient for producing a beneficial result whereas an “immunologically effective amount” corresponds to the amount of each active entity that is sufficient for producing a detectable immune response.

### Therapeutic Vaccine

Any type of therapeutic vaccines can be used in the context of the invention including, but not limited to, cell-based vaccines, peptide or polypeptide-based vaccines, microorganism-based vaccines and vector-based vaccines. Cell based vaccines typically rely on cells (e.g. cancer cells, immune cells and stem cells) obtained from a patient which are in vitro treated and then re-introduced in vivo (e.g. in the same patient or a group of patients). For example, specialized cells such as immune cells (e.g. Tumor Infiltrating Lymphocytes (TIL) or dendritic cells (DC)) or cancer cells can be collected from a subject, optionally treated in vitro (e.g. irradiated cancer cells) and reprogramed in vitro to be more amenable to the host's immune system before being reinfused into a patient's bloodstream. Representative examples include but are not limited to the vaccine developed by Immunocellular Therapeutics targeting six tumor-associated antigens (TAA) involved in glioblastoma, and the DC-based Provenge® vaccine (sipuleucel-T) approved for treating advanced prostate cancer.

Polypeptide-based vaccines can be generated by recombinant or synthetic means. Exemplary polypeptide-based vaccines suitable in the context of the invention include, without limitation, the liposomal vaccine Stimuvax® which incorporates lipopeptides generated from the mucin 1 (MUC1) glycoprotein and showed some beneficial effects in some subgroups of patients with advanced non-small cell lung cancer (NSCLC); Newax E75 developed by Galena and Genentech for breast cancer SL-701, a synthetic multipeptide vaccine developed by Stemline Therapeutics for treating glioma brain tumors; and monoclonal antibodies that are now conventionally used in clinics to attack specific types of diseased cells (e.g. the anti-CD20 rituximab approved for treatment of non-Hodgkins lymphomas, trastuzumab for the treatment of breast cancer with HER2/neu overexpression and bevacizumab that target VEGF and can be used as antiangiogenic cancer therapy). Such polypeptide-based vaccines can be used in connection with adjuvants if needed. Adjuvants are known in the art.

Microorganism-based therapeutic vaccines typically employ avirulent or attenuated microorganisms which optionally have been engineered for expressing polypeptides of interest. Well-known examples of suitable microorganisms include without limitation bacterium (e.g. Mycobacterium; Lactobacillus (e.g. Lactococcus lactis); Listeria (e.g. Listeria monocytogenes) Salmonella and Pseudomona) and yeast (e.g. Saccharomyces cerevisiae, Schizosaccharomyces pombe, Pichia pastoris). A suitable bacterium therapeutic vaccine is Mycobacterium bovis (BCG) widely used for treating bladder cancer and a suitable yeast therapeutic vaccine is Tarmogens® developed by Globelmmune made from genetically-modified yeast that express one or more disease-associated antigens.

In a preferred embodiment, the therapeutic vaccine in use in this invention is a vector-based therapeutic vaccine (or vectorized therapeutic vaccine) that typically, comprises a plasmid or a viral vector (live, inactivated, attenuated, killed, oncolytic, etc.). The term “vector” as used herein refers to a vehicle, preferably a polynucleotide (plasmid DNA, viral vector, etc.) or a viral particle that contains the elements necessary to allow delivery, propagation and/or expression of biological substances within a host cell or subject. This term encompasses extrachromosomal vectors (e.g. that remain in the cell cytosol or nucleus) and integration vectors (e.g. designed to integrate into the cell genome) as well as cloning vectors, shuttle vectors (e.g. functioning in both prokaryotic and/or eukaryotic hosts), transfer vectors (e.g. for transferring nucleic acid molecule(s) in a viral genome) and expression vectors for expression in various host cells or organisms. For the purpose of the invention, the vectors may be of naturally occurring genetic sources, synthetic or artificial, or some combination of natural and artificial genetic elements.

A “plasmid” as used herein refers to a replicable DNA construct. Usually plasmid vectors contain selectable marker genes that allow host cells carrying the plasmid vector to be selected for or against in the presence of a corresponding selective drug. A variety of positive and negative selectable marker genes are known in the art. By way of illustration, an antibiotic resistance gene can be used as a positive selectable marker gene that allows selection of the plasmid-containing cells in the presence of the corresponding antibiotic. Suitable plasmid vectors include, without limitation, pREP4, pCEP4 (Invitrogene), pCl (Promega), pCDM8 (Seed, 1987, Nature 329: 840), pMT2PC (Kaufman et al., 1987, EMBO J. 6: 187-95), pVAX (Invitrogen) and pgWiz (Gene Therapy System Inc; Himoudi et al., 2002, J. Virol. 76: 12735-46).

In a more preferred embodiment, the therapeutic vaccine for use in the present invention comprises a viral vector. In the context of the invention, the term “viral vector” as used herein refers to a vector that includes at least one element of a virus genome allowing packaging into a viral particle. This term has to be understood broadly as including nucleic acid vector (RNA or DNA) as well as viral particles generated thereof, and especially infectious viral particles. The term “infectious” refers to the ability of a viral vector to infect and enter into a host cell or subject.

Viral vectors can be replication-competent or selective (e.g. engineered to replicate better or selectively in specific host cells), or can be genetically disabled so as to be replication-defective or replication-impaired. Viral vectors can be engineered from a variety of viruses and in particular from the group of viruses consisting of adenovirus, poxvirus, adenovirus-associated virus (AAV), herpes virus (HSV), measles virus, foamy virus, alphavirus, vesicular stomatis virus, Newcastle disease virus, picorna virus, Sindi virus, etc. One may use either wild-type strains as well as derivatives thereof (i.e. a virus that is modified compared to the wild-type strain, e.g. by truncation, deletion, substitution, and/or insertion of one or more nucleotide(s) contiguous or not within the viral genome).

Modification(s) can be within endogenous viral genes (e.g. coding and/or regulatory sequences) and/or within intergenic regions. Moreover, modification(s) can be silent or not (e.g. resulting in a modified viral gene product). Modification(s) can be made in a number of ways known to those skilled in the art using conventional molecular biology techniques.

Preferably, the modifications encompassed by the present invention affect, for example, virulence, toxicity, pathogenicity or replication of the virus compared to a virus without such modification, but do not completely inhibit infection and production at least in permissive cells. Said modification(s) preferably lead(s) to the synthesis of a defective protein (or lack of synthesis) so as to be unable to ensure the activity of the protein produced under normal conditions by the unmodified gene. Exemplary modifications are disclosed in the literature with a specific preference for those altering viral genes involved in DNA metabolism, host virulence and IFN pathway (see e.g. Guse et al., 2011, Expert Opinion Biol. Ther. 11(5):595-608). Other suitable modifications include the insertion of exogenous gene(s) (e.g. nucleic acid molecule(s) of interest) as described hereinafter.

In a preferred embodiment, the therapeutic vaccine comprised in the combination of the invention is a replication-defective or replication-impaired viral vector which means that it cannot replicate to any significant extent in normal cells, especially in normal human cells. The impairment or defectiveness of replication functions can be evaluated by conventional means, such as by measuring DNA synthesis and/or viral titer in non-permissive cells. The viral vector can be rendered replication-defective by partial or total deletion or inactivation of regions critical to viral replication. Such replication-defective or impaired viral vectors typically require for propagation, permissive host cells which bring up or complement the missing/impaired functions.

In one embodiment, the viral vector for use in the present invention is obtained from a poxvirus. As used herein the term “poxvirus” refers to a virus belonging to the Poxviridae family with a preference for the Chordopoxvirinae subfamily directed to vertebrate host which includes several genus such as Orthopoxvirus, Capripoxvirus, Avipoxvirus, Parapoxvirus, Leporipoxvirus and Suipoxvirus. Orthopoxviruses are preferred in the context of the present invention as well as the Avipoxviruses including Canarypoxvirus (e.g. ALVAC) and Fowlpoxvirus (e.g. the FP9 vector). In a preferred embodiment, the therapeutic vaccine comprises a poxviral vector belonging to the Orthopoxvirus genus and even more preferably to the vaccinia virus (VV) species. Any vaccinia virus strain can be used in the context of the present invention including, without limitation, Western Reserve (WR), Copenhagen (Cop), Lister, LIVP, Wyeth, Tashkent, Tian Tan, Brighton, Ankara, MVA (Modified vaccinia virus Ankara), LC16M8, LC16M0 strains, etc. with a specific preference for WR, Copenhagen, Wyeth and MVA vaccinia virus. Sequences of the genome of various Poxviridae, are available in the art in specialized databanks such as Genbank (e.g. accession numbers NC_006998, M35027, NC_005309, U94848 provide sequences of WR, Copenhagen, Canarypoxvirus and MVA genomes).

The poxvirus for use in this invention can be engineered for various purposes, e.g. improved safety (e.g. attenuation) and/or efficacy (e.g. improved selectivity for cancer cells and/or decreased toxicity in healthy cells). A number of viral genes are suitable for such modifications, such as the thymidine kinase (J2R, Genbank accession number AAA48082), the deoxyuridine triphosphatase (F2L), the viral hemagglutinin (A56R); the small (F4L) and/or the large (I4L) subunit of the ribonucleotide reductase, the serine protease inhibitor (B13R/B14R) and the complement 4b binding protein (C3L). Representative examples of suitable VV for use in this invention include NYVAC (U.S. Pat. No. 5,494,807) as well as TK-defective, TK- and F2L-defective (WO2009/065547) and TK- and I4L-defective VV (WO2009/065546). The gene nomenclature used herein is that of Copenhagen Vaccinia strain. It is also used herein for the homologous genes of other poxviridae unless otherwise indicated. However, gene nomenclature may be different according to the pox strain but correspondence between Copenhagen and other vaccinia strains are generally available in the literature.

A particularly appropriate viral vector for use in the context of the present invention is MVA due to its highly-attenuated phenotype (Mayr et al., 1975, Infection 3: 6-14; Sutter and Moss, 1992, Proc. Natl. Acad. Sci. USA 89: 10847-51), a more pronounced IFN-type 1 response generated upon infection compared to non-attenuated vectors and availability of the sequence of its genome in the literature (Antoine et al., 1998, Virol. 244: 365-96 and Genbank accession number U94848).

In one embodiment, the viral vector for use in the present invention is obtained from a paramyxoviridae and especially from a morbillivirus such as measles. Various attenuated strains are available in the art, such as and without limitation, the Edmonston A and B strains (Griffin et al., 2001, Field's in Virology, 1401-1441), the Schwarz strain (Schwarz A, 1962, Am J Dis Child, 103: 216), the 5-191 or C-47 strains (Zhang et al., 2009, J Med Virol. 81 (8): 1477). One may also use recombinant Newcastle Disease Virus (NDV) (Bukreyev and Collins, 2008, Curr Opin Mol Ther 10: 46-55) with a specific preference for an attenuated strain thereof such as MTH-68 that was already used in cancer patients (Csatary et al., 1999, Anti Cancer Res 19: 635-8) and NDV-HUJ, which showed promising results in glioblastoma patients (isracast.com Mar. 1, 2006).

In one embodiment, the viral vector for use in the present invention is obtained from a herpes simplex virus (HSV). The Herpesviridae are a large family of DNA viruses that all share a common structure and are composed of relatively large double-stranded, linear DNA genomes encoding 100-200 genes encapsided within an icosahedral capsid which is enveloped in a lipid bilayer membrane. Although the oncolytic herpes virus can be derived from different types of HSV, particularly preferred are HSV1 and HSV2. The herpes virus may be genetically modified so as to restrict viral replication in tumors or reduce its cytotoxicity in non-dividing cells. For example, any viral gene involved in nucleic acid metabolism may be inactivated, such as thymidine kinase (Martuza et al., 1991, Science 252: 854-6), ribonucleotide reductase (RR) (Boviatsis et al., 1994, Gene Ther. 1: 323-31; Mineta et al., 1994, Cancer Res. 54: 3363-66), or uracil-N-glycosylase (Pyles et al., 1994, J. Virol. 68: 4963-72). Another aspect involves viral mutants with defects in the function of genes encoding virulence factors such as the ICP34.5 gene (Chambers et al., 1995, Proc. Natl. Acad. Sci. USA 92: 1411-5). Representative examples of oncolytic herpes virus include NV1020 (e.g. Geevarghese et al., 2010, Hum. Gene Ther. 21(9): 1119-28) and T-VEC (Andtbacka et al., 2013, J. Clin. Oncol. 31, abstract number LBA9008).

In one embodiment, the viral vector for use in the present invention is obtained from an adenovirus. The term “adenovirus” (or Ad) refers to a group of viruses belonging to the Adenoviridae family. Generally speaking, adenoviruses are non-enveloped and their genome consists of a single molecule of linear, double stranded DNA that codes for more than 30 proteins including the regulatory early proteins participating in the replication and transcription of the viral DNA which are distributed in 4 regions designated E1 to E4 (E denoting “early”) dispersed in the adenoviral genome and the late (L) structural proteins (see e.g. Evans and Hearing, 2002, in “Adenoviral Vectors for Gene Therapy” pp 39-70, eds. Elsevier Science). E1, E2 and E4 are essential to the viral replication whereas E3 is dispensable and appears to be responsible for inhibition of the host's immune response in the course of adenovirus infection.

Adenoviral vectors for use herein can be obtained from a variety of human or animal adenoviruses (e.g. canine, ovine, simian, etc.) and any serotype can be employed. It can also be a chimeric adenovirus (WO2005/001103). One of skill will recognize that elements derived from multiple serotypes can be combined in a single adenovirus.

Desirably, the adenoviral vector originates from a human Ad, including those of rare serotypes, or from a primate (e.g. chimpanzee, gorilla). Representative examples of human adenoviruses include subgenus C (e.g. Ad2 Ad5 and Ad6), subgenus B (e.g. Ad3, Ad7, Ad11, Ad14, Ad34, Ad35 and Ad50), subgenus D (e.g. Ad19, Ad24, Ad26, Ad48 and Ad49) and subgenus E (Ad4). Representative examples of chimp Ad include without limitation AdCh3 (Peruzzi et al., 2009, Vaccine 27: 1293-300) and AdCh63 (Dudareva et al, 2009, Vaccine 27: 3501-4) and any of those described in the art (see for example, WO2010/086189; WO2009/105084; WO2009/073104; WO2009/073103; WO2005/071093; and WO03/046124). An exemplary genome sequence of human adenovirus type 5 (Ad5) is found in GenBank Accession M73260 and in Chroboczek et al. (1992, Virol. 186: 280-5).

Preferably, the adenovirus employed in this invention is replication-defective, e.g. by total or partial deletion of E1 region. An appropriate E1 deletion extends from approximately positions 459 to 3510 by reference to the sequence of the Ad5 disclosed in the GenBank under the accession number M 73260. The adenoviral genome may comprise additional modification(s) (e.g. deletion of all or part of other essential E2 and/or E4 regions as described in WO94/28152; Lusky et al, 1998, J. Virol 72: 2022). In addition, the non-essential E3 region can also be mutated or deleted.

More preferably, the adenovirus comprised in the therapeutic vaccine of the invention is a human adenovirus of serotype 5 (Ad5), defective for E1 and/or E3 function and comprising a nucleic acid molecule encoding a polypeptide of interest inserted in the E1 region.

The present invention also encompasses therapeutic vaccines complexed to lipids or polymers (e.g. polyethylene glycol) to form particulate structures such as liposomes, lipoplexes or nanoparticles as well as targeted ones modified to allow preferential targeting to a specific host cell. Targeting can be carried out through genetic means (e.g. by genetically inserting a ligand capable of recognizing and binding to a cellular and surface-exposed component into a polypeptide present on the surface of the virus) or by chemical means (e.g. by modifying a viral surface envelope). Examples of suitable ligands include antibodies or fragments thereof directed to cell-specific, tissue-specific and pathogen-associated markers.

### Recombinant Therapeutic Vaccines

In one embodiment, the therapeutic vaccine for use herein is recombinant in the sense that it has been engineered to deliver in situ and thus contains or encodes one or more polypeptide(s) of interest. Such one or more polypeptide(s) of therapeutic interest can compensate for pathological symptoms, e.g. by acting through toxic effects to limit or remove harmful cells from the body (e.g. a suicide gene product) or by acting as target polypeptide for an immune response (e.g. an antigen) or by improving the host's immune system (e.g. a cytokine). Such polypeptides can be obtained from a natural source—of mammal origin (e.g. human) or not (e.g. from a pathogen)—or be altered in lab (so as to include suitable sequence modification(s)) and can be produced by synthetic means or by a biological process (e.g. recombinantly produced). As mentioned above, the present invention encompasses the use/expression of native polypeptide(s) as well as fragments and analogs thereof.

### Suicide Gene Products

The term “suicide gene” refers to a nucleic acid molecule coding for a protein (e.g. enzyme) able to convert a precursor of a drug into a cytotoxic compound. Appropriate suicide genes for use in this invention are disclosed in the following Table with the corresponding prodrug (or drug precursor) and the active (cytotoxic) drug.

TABLE 1

| Enzyme | Prodrug | Active Drug |
|---|---|---|
| Thymidine phosphorylase | 5-FU | 5-FdUMP |
| | 5′-DFUR | 5-FU |
| Deoxycitidine kinase | Gemcitabine | Gemcitabine monophosphate |
| Cytidine deaminase | 5′-DFCR | 5′-DFUR |
| Cytosine deaminase | 5-FC | 5-FU |
| Uracil | 5-FU | 5-FUMP |
| phosphoribosyltransferase | | |
| Thymidine phosphorylase | 5-FU | 5-FdUMP |
| Thymidine kinase (HSV) | Ganciclovir | Ganciclovir-triphosphate |
| | | nucleotide |
| Nitroreductase | CB1954 | 5-(Aziridin-1-yl)-4-hydroxyl- |
| | | amino-2-nitro-benzamide |
| Cytochrome P450 | Ifosfamide | Isophosphoramide mustard |
| | Cyclophosmamide | Phosphoramide mustard |
| Purine-nucleoside | Fludarabine | 2-Fluoroadenine |
| phosphorylase | | |
| Alkaline phosphatase | Etoposide phosphate | Etoposide |
| | Mitomycin C phosphate | Mitomycin C |
| | N-(4-phophonooxy- | Doxorubicin |
| | phenylacetyl)doxorubicin | |
| Carboxypeptidase | Methotrexate-amino acids | Methotrexate |
| Penicillin amidase | N-(phenylacetyl) doxorubicin | Doxorubicin |
| β-Lactamase | C-DOX | Doxorubicin |

Desirably, the therapeutic vaccine comprises or encodes a polypeptide having at least cytosine deaminase (CDase) activity. CDase encoding nucleic acid molecules can be obtained from any prokaryotes and lower eukaryotes such as Saccharomyces cerevisiae (FCY1 gene), Candida Albicans (FCA1 gene) and Escherichia coli (codA gene). Alternatively or in combination, the therapeutic vaccine comprises or encodes a polypeptide having uracil phosphoribosyl transferase (UPRTase) activity. UPRTase-encoding nucleic acid molecules can be obtained from E. coli (Andersen et al., 1992, European J. Biochem. 204: 51-56), Lactococcus lactis (Martinussen et al., 1994, J. Bacteriol. 176: 6457-63), Mycobacterium bovis (Kim et al., 1997, Biochem. Mol. Biol. Internat. 41: 1117-24), Bacillus subtilis (Martinussen et al., 1995, J. Bacteriol. 177: 271-4) and yeast (e.g. S. cerevisiae FUR1 disclosed by Kern et al., 1990, Gene 88: 149-57). The nucleotide sequence of such CDase and UPRTase-encoding nucleic acid molecules and amino acids of the encoded enzyme are also available in specialized data banks (SWISSPROT EMBL, Genbank, Medline and the like).

Functional analogues may also be used. It is within the reach of the skilled person to engineer analogs from the published data, and test the enzymatic activity in an acellular or cellular system according to conventional techniques (see e.g. EP998568). For illustrative purposes, suitable functional analogues comprise the N-terminally truncated FUR1 mutant described in EP998568 (with a deletion of the 35 first residues up to the second Met residue present at position 36 in the native protein) which exhibits a higher UPRTase activity than that of the native enzyme as well as the FCY1::FUR1 fusions named FCU1 (amino acid sequence represented in the sequence identifier SEQ ID NO: 1 of OW2009/065546) and FCU1-8 described in WO96/16183, EP998568 and WO2005/07857.

### Cytokines

Typically, a cytokine works by signal transduction to control the immune system and its effector cells. Examples of suitable cytokines include without limitation interleukins (e.g. IL-2, IL-6, IL-7, IL-12, IL-15, IL-24), chemokines (e.g. CXCL10, CXCL9, CXCL11), interferons (e.g. IFNα, IFNβ, IFNγ), tumor necrosis factor (TNF), colony-stimulating factors (e.g. GM-CSF, C-CSF, M-CSF . . . ), APC (for Antigen Presenting Cell)-exposed proteins (e.g. B7.1, B7.2 and the like), growth factors (Transforming Growth Factor TGF, Fibroblast Growth Factor FGF, Vascular Endothelial Growth Factors VEGF, and the like), major histocompatibility complex (MHC) antigens of class I or II, apoptosis inducers or inhibitors (e.g. Bax, BcI2, BcIX . . . ). Preferably, the cytokine is an interleukin or a colony-stimulating factor (e.g. GM-CSF).

### Antigens

In one embodiment, the therapeutic vaccine comprised in the first composition for use herein may comprise or encode any antigen. The term “antigen” generally refers to a substance that is recognized and selectively bound by an antibody or by a T cell antigen receptor, in order to trigger an immune response. It is contemplated that the term antigen encompasses native antigen as well as fragment (e.g. epitopes, immunogenic domains, etc.) and derivative thereof, provided that such fragment or derivative is capable of being the target of an immune response. Suitable antigens include, but not limited to, biological components (e.g. peptides, polypeptides, post translational modified polypeptides and polynucleotides); complex components (e.g. cells, cell mixtures, live or inactivated organisms such as bacteria, viruses, fungi, prions, etc . . . ), and combinations thereof. In a preferred embodiment of the invention, the antigen comprised or expressed by the therapeutic vaccine comprised in the first composition is a polypeptide including one or more B cell epitope(s) or one or more T cell epitope(s) or both B and T cell epitope(s) and capable of raising an immune response, preferably, a humoral or cell response that can be specific for that antigen including a CD4 T cell response (e.g., Th1, Th2 and/or Th17) and/or a CD8+ T cell response (e.g., a CTL response). A vast variety of direct or indirect biological assays are available in the art to evaluate the immunogenic nature of an antigen either in vivo (animal or human being), or in vitro (e.g. in a biological sample) as described herein.

Some embodiments also contemplate the expression from the therapeutic vaccine of fusion polypeptides. The term “fusion” or “fusion protein” as used herein refers to the combination of two or more polypeptides/peptides in a single polypeptide chain. The fusion can be direct (i.e. without any additional amino acid residues in between) or through a linker (e.g. 3 to 30 amino acids long peptide composed of amino acid residues such as glycine, serine, threonine, asparagine, alanine and/or proline). It is within the reach of the skilled person to define accordingly the need and location of the translation-mediating regulatory elements (e.g. the initiator Met and codon STOP). For example, multiepitopes from the same or different antigen(s) may be envisaged as well. Typically, the one or more antigen(s) is selected in connection with the disease to treat. Preferred antigens for use herein are cancer antigens and antigens of pathogens.

In certain embodiments, the antigen(s) contained in or encoded by the therapeutic vaccine is/are cancer antigen(s) (also called tumor-associated antigens). As used herein, the term “cancer antigen” refers to a polypeptide and the like, that is associated with and/or serve as markers for cancers. Cancer antigens encompass various categories of polypeptides, e.g. those which are normally silent (i.e. not expressed) in normal cells, those that are expressed only at low levels or at certain stages of differentiation and those that are temporally expressed such as embryonic and foetal antigens as well as those resulting from mutation of cellular genes, such as oncogenes (e.g. activated ras oncogene), proto-oncogenes (e.g. ErbB family), or proteins resulting from chromosomal translocations. The cancer antigens also encompass antigens encoded by pathogenic organisms (bacteria, viruses, parasites, fungi, viroids or prions) that are capable of inducing a malignant condition in a subject (especially chronically infected subject) such as RNA and DNA tumor viruses (e.g. HPV, HCV, HBV, EBV, etc.) and bacteria (e.g. Helicobacter pilori).

Some non-limiting examples of cancer antigens include, without limitation, MART-1/Melan-A, gp100, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family (e.g. MUC1, MUC16, etc.; see e.g. U.S. Pat. No. 6,054,438; WO98/04727; or WO98/37095), HER2/neu, p21ras, RCAS1, alpha-fetoprotein, E-cadherin, alpha-catenin, beta-catenin and gamma-catenin, p120ctn, gp100.sup.Pmel117, PRAME, NY-ESO-1, cdc27, adenomatous polyposis coli protein (APC), fodrin, Connexin 37, Ig-idiotype, p15, gp75, GM2 and GD2 gangliosides, Smad family of cancer antigens brain glycogen phosphorylase, SSX-1, SSX-2 (HOM-MEL-40), SSX-1, SSX-4, SSX-5, SCP-1 and CT-7, and c-erbB-2 and viral antigens such as the HPV-16 and HPV-18 E6 and E7 antigens and the EBV-encoded nuclear antigen (EBNA)-1.

Alternatively or in combination with the cancer antigens embodiment, the therapeutic vaccine includes or encodes one or more antigen(s) originating from an infectious organism or associated with a disease or condition caused by an infectious organism. Such antigens include, but are not limited to, viral antigens, fungal antigens, bacterial antigens, parasitic antigens and protozoan antigens.

Other antigens suitable for use in this invention are marker antigens (beta-galactosidase, luciferase, green fluorescent proteins, etc.).

The present invention also encompasses therapeutic vaccine comprising/expressing two or more polypeptides of interest as described above, e.g. at least two antigens, at least one antigen and one cytokine, at least two antigens and one cytokine, etc.

A preferred therapeutic vaccine comprised in the immunostimulatory combination of the invention or for use according to this invention comprises or encodes one or more polypeptides of interest selected from the group consisting of:
A mucin antigen (e.g. MUC-1)
HPV antigen(s), in particular non-oncogenic E6 and E7 antigen;
HCV antigen(s) (e.g. the non-structural antigens NS3, NS4 and/or NS5 described in WO2004/111082);
HBV antigen(s) (e.g. the core, polymerase, the X antigen and/or the HBs antigen);
Mycobacterium (Mtb) antigen(s) (e.g. any of those described in WO2014/009438);
The human IL-2;
The human GM-CSF;
The FCU-1 suicide gene; and.
any combination thereof.

When the native polypeptide of interest exerts undesired properties (e.g. oncogenic or 30 transforming properties, cytotoxicity, etc.), it may be advantageous to mutate the polypeptide. For example, to circumvent oncogenicity of HPV E6 and E7 polypeptides, one may use or express non-oncogenic analogs displaying reduced capacity to bind p53 and Rb, respectively. Such non-oncogenic analogs are described in WO99/03885. For illustrative purpose, a non-oncogenic HPV-16 E6 variant may be generated by deletion of residues 118 to 122 (CPEEK) whereas a non-oncogenic HPV-16 E7 variant can be deleted of residues 21 to 26 (DLYCYE) (+1 representing the first methionine residue of the native HPV polypeptide.

Another preferred embodiment of this invention comprises an HBV-targeted therapeutic vaccine encoding one or more antigen(s) originating from a hepatitis B virus, and more preferably from a human hepatitis B virus (HBV). As used herein, “hepatitis B virus” refers to any member of the Hepadnaviridae (see e.g. Ganem and Schneider in Hepadnaviridae (2001) “The viruses and their replication”, pp2923-2969, Knipe DM et al, eds. Fields Virology, 4th ed. Philadelphia, Lippincott Williams & Wilkins or subsequent edition). Typically, Hepadnaviruses are small enveloped hepatotropic DNA viruses having a partially double-stranded, circular DNA of approximately 3,200 nucleotides with a compact gene organization. More specifically, the HBV genome contains 4 overlapping open reading frames (ORFs), C, S, P and X. The C ORF encodes the core protein (or HBc) constitutive of the nucleocapsid, the S ORF the envelop proteins, the P ORF the viral polymerase and the X ORF a protein known as the X protein which is thought to be a transcriptional activator. In accordance with the present invention, the encoded HBV antigen(s) can be independently native (i.e. naturally-occurring) or modified (e.g. analogs or fragments of native HBV antigens). Although the one or more HBV antigens for use herein encoded HBV antigens may originate from distinct HBV, especially from distinct genotypes, it is preferred that they all originate from a genotype D HBV virus, with a specific preference for HBV isolate Y07587 (Genbank accession number Y07587 and Stoll-Becker et al, 1997, J. Virol. 71: 5399). A particularly preferred embodiment is directed to a fusion comprising (i) a core antigen; (ii) a polymerase antigen and (iii) one or more HBsAg immunogenic domain(s) with a specific preference for a fusion comprising at its N-terminus, a C-term truncated core (e.g. positions 1 to 148 of a native HBc with the initiator Met) fused to a pol antigen (without initiator Met) having two env immunogenic domain inserted within pol in place of some residues involved in polymerase activity and some residues involved in RNaseH activity. More preferred is a fusion protein as described in WO2013/007772 and even more preferred an HBV antigen fusion protein comprising an amino acid sequence which exhibits at least 80% of identity with the amino acid sequence shown in SEQ ID NO: 17.

Other suitable structural features may be used with the polypeptide(s) of interest to improve its cloning, synthesis, processing, stability, solubility and/or efficacy. For example, membrane anchorage of the polypeptide(s) of interest may be used to improve MHC class I and/or MHC class II presentation. Membrane presentation can be achieved by incorporating in the polypeptide of interest a membrane-anchoring sequence and a secretory sequence (i.e. a signal peptide) if the native polypeptide lacks it. Briefly, signal peptides usually comprise 15 to 35 essentially hydrophobic amino acids which are then removed by a specific ER (endoplasmic reticulum)-located endopeptidase to give the mature polypeptide. Trans-membrane peptides are also highly hydrophobic in nature and serve to anchor the polypeptides within cell membrane. Appropriate trans-membrane and/or signal peptides are known in the art. They may be obtained from cellular or viral polypeptides such as those of immunoglobulins, tissue plasminogen activator, insulin, rabies glycoprotein, the HIV virus envelope glycoprotein or the measles virus F protein or may be synthetic. Preferably, the secretory sequence is inserted at the N-terminus of the polypeptide downstream of the codon for initiation of translation and the membrane-anchoring sequence at the C-terminus, preferably immediately upstream of the stop codon. A preferred example is illustrated by an HBV fusion protein comprising an amino acid sequence which exhibits at least 80% of identity with the amino acid sequence shown in SEQ ID NO: 18.

### Polypeptide-Encoding Nucleic Acid Molecule and Generation of Vectorised Therapeutic Vaccine

The nucleic acid molecule encoding a polypeptide of interest for use herein can independently be generated by a number of ways known to those skilled in the art (e.g. cloning, PCR amplification, DNA shuffling). For example, the polypeptide-encoding nucleic acid molecule can be isolated independently from any available source (e.g. biologic materials described in the art such as cDNA, genomic libraries, viral genomes or any prior art vector known to include it) using sequence data available to the skilled person and the sequence information provided herein, and then suitably inserted in the vectorised therapeutic vaccine by conventional molecular biology techniques. Alternatively, the polypeptide-encoding nucleic acid molecule can also be generated by chemical synthesis in automatized process (e.g. assembled from overlapping synthetic oligonucleotides or synthetic gene). Preferably, such a nucleic acid molecule of interest is obtained from cDNA and does not comprise intronic sequences. Modification(s) can be generated by a number of ways known to those skilled in the art, such as chemical synthesis, site-directed mutagenesis, PCR mutagenesis, etc.

In particular, it might be advantageous to optimize the nucleic acid sequence for providing high level expression in a particular host cell or subject. It has been indeed observed that, the codon usage patterns of organisms are highly non-random and the use of codons may be markedly different between different hosts. As the polypeptide of interest may be from prokaryote (e.g. bacterial or viral antigen) or lower eukaryote (e.g. the suicide gene) origin, its coding sequence may have an inappropriate codon usage pattern for efficient expression in higher eukaryotic cells (e.g. human). Typically, codon optimization is performed by replacing one or more “native” codon corresponding to a codon infrequently used by one or more codon encoding the same amino acid which is more frequently used in the subject to treat. It is not necessary to replace all native codons corresponding to infrequently used codons since increased expression can be achieved even with partial replacement.

Further to optimization of the codon usage, expression can also be improved through additional modifications of the nucleotide sequence. For example, the nucleic acid sequence can be modified so as to prevent clustering of rare, non-optimal codons being present in concentrated areas and/or to suppress or modify “negative” sequence elements which are expected to negatively influence expression levels. Such negative sequence elements include without limitation the regions having very high (>80%) or very low (<30%) GC content; AT-rich or GC-rich sequence stretches; unstable direct or inverted repeat sequences; and/or internal cryptic regulatory elements such as internal TATA-boxes, chi-sites, ribosome entry sites, and/or splicing donor/acceptor sites.

Moreover, when homologous nucleic acid molecules are to be expressed, such homologous sequences can be degenerated over the full length nucleic acid molecule or portion(s) thereof so as to reduce sequence homology. It is indeed advisable to degenerate the portions of nucleic acid sequences that show a high degree of sequence identity (e.g. the same antigen obtained from various serotypes of a given pathogen) so as to avoid homologous recombination problems during production process and the skilled person is capable of identifying such portions by sequence alignment.

For the purposes of the present invention, the nucleic acid molecule(s) encoding the polypeptide(s) of interest can be inserted or included in the therapeutic vaccine according to the conventional practice in the art. Typically, with regard to viral vectors, the nucleic acid molecule(s) of interest is/are preferably inserted within a viral gene, an intergenic region, in a non-essential gene or region or in place of viral sequences. The general conditions for constructing and producing recombinant poxviruses are well known in the art (see for example WO2010/130753; WO03/008533; U.S. Pat. Nos. 6,998,252; 5,972,597 and 6,440,422). The nucleic acid molecule(s) of interest is/are preferably inserted within the poxviral genome in a non-essential locus. Thymidine kinase gene is particularly appropriate for insertion in Copenhagen vaccinia vectors and deletion II or III for insertion in MVA vector (WO97/02355; Meyer et al., 1991, J. Gen. Virol. 72: 1031-8). The general conditions for constructing and producing recombinant measles viruses are well known in the art. Insertion of the nucleic acid molecule(s) of interest between P and M genes or between H and L genes is particularly appropriate. The general conditions for constructing and producing recombinant adenoviruses are well known in the art (see e.g. Chartier et al., 1996, J. Virol. 70: 4805-10 and WO96/17070). E1 or E3 region is the preferred site of insertion for the nucleic acid molecule(s) to be expressed which can be positioned in sense or antisense orientation relative to the natural transcriptional direction of the region in question.

In one embodiment, the one or more polypeptide(s) of interest are encoded in one or more vector(s) in the same or independent site of insertion, resulting in a single or multi vector first composition.

In a particularly preferred embodiment, the therapeutic vaccine is selected from the group consisting of:
A MVA virus encoding the MUC-1 antigen and human IL-2 as represented by TG4010 described in WO92/07000, U.S. Pat. No. 5,861,381 and Limacher and Quoix (2012, Oncolmmunology 1(5): 791-2);
A MVA virus encoding membrane anchored HPV-16 non-oncogenic E6 and E7 antigens and human IL-2 as represented by TG4001 described in WO99/03885;
A MVA virus encoding the FCU1 gene as represented by TG4023 (WO99/54481);
A vaccinia virus encoding the FCU1 gene as represented by TG6002 (as described in WO2009/0655546);
A MVA virus encoding one or more Mtb antigens (see e.g. WO2014/009438 and WO2015/104380); and
An Ad (e.g. Ad5) virus encoding a fusion of HBV HBc, pol, and one or more env immunogenic domain(s) such as envl and env2 (corresponding to the portions of residues 14-51 and 165-194 of HBsAg), especially a fusion as represented by TG1050 (also named AdTG18201 as described in WO2013/007772).

### Expression of the Nucleic Acid Molecule(s) Encoding the Polypeptide(s) of Interest

In accordance with the present invention, the nucleic acid molecule(s) expressed by the therapeutic vaccine comprised in the first composition is/are operably linked to suitable regulatory elements for expression in the desired host cell or subject.

As used herein, the term “regulatory elements” or “regulatory sequence” refers to any element that allows, contributes or modulates the expression of the nucleic acid molecule(s) in a given host cell or subject, including replication, duplication, transcription, splicing, translation, stability and/or transport of the nucleic acid(s) or its derivative (i.e. m RNA). As used herein, “operably linked” means that the elements being linked are arranged so that they function in concert for their intended purposes. For example, a promoter is operably linked to a nucleic acid molecule if the promoter effects transcription from the transcription initiation to the terminator of said nucleic acid molecule in a permissive host cell. It will be appreciated by those skilled in the art that the choice of the regulatory sequences can depend on factors such as the nucleic acid molecule(s) itself, the vector from which it is expressed, the level of expression desired, etc.

The promoter is of special importance. In the context of the invention, it can be constitutive directing expression of the nucleic acid molecule(s) in many types of cells or specific to certain types of cells or tissues or regulated in response to specific events or exogenous factors (e.g. by temperature, nutrient additive, hormone, etc.) or according to the phase of a viral cycle (e.g. late or early). One may also use promoters that are repressed during the production step in response to specific events or exogenous factors, in order to optimize production of the therapeutic vaccine and circumvent potential toxicity of the expressed polypeptide(s).

Suitable constitutive promoters for expression in recombinant adenovirus and plasmid vectors include, but are not limited to, the cytomegalovirus (CMV) immediate early promoter (U.S. Pat. No. 5,168,062), the RSV promoter, the adenovirus major late promoter, the phosphoglycero kinase (PGK) promoter (Adra et al., 1987, Gene 60: 65-74), the thymidine kinase (TK) promoter of herpes simplex virus (HSV)-1 and the T7 polymerase promoter (WO98/10088). Vaccinia virus promoters are particularly adapted for expression in recombinant poxviruses. Representative examples include without limitation the vaccinia 7.5K, HSR, 11K7.5 (Erbs et al., 2008, Cancer Gene Ther. 15(1): 18-28), TK, pB2R, p28, p11 and KU promoter, as well as synthetic promoters such as those described in Chakrabarti et al. (1997, Biotechniques 23: 1094-7; Hammond et al, 1997, J. Virol Methods 66: 135-8; and Kumar and Boyle, 1990, Virology 179: 151-8) as well as early/late chimeric promoters. Promoters suitable for measles viruses include without limitation any promoter directing expression of measles transcription units (Brandler and Tangy, 2008, CIMID 31: 271).

Those skilled in the art will appreciate that the regulatory elements controlling the expression of the nucleic acid molecule(s) of interest may further comprise additional elements for proper initiation, regulation and/or termination of transcription (e.g. polyA transcription termination sequences), mRNA transport (e.g. nuclear localization signal sequences), processing (e.g. splicing signals), and stability (e.g. introns and non-coding 5′ and 3′ sequences), translation (e.g. an initiator Met, tripartite leader sequences, IRES ribosome binding sites, signal peptides, etc.) and purification steps (e.g. a tag). In a preferred embodiment, the therapeutic vaccine for use in the invention comprises a MVA vector which contains inserted into its genome (preferably in deletion II) a nucleic acid molecule encoding a tumor-associated antigen such as MUC-1 (preferably under the transcriptional control of the early/late vaccinia pH5R promoter) and a nucleic acid molecule encoding a cytokine such as the human IL-2 (preferably under the transcriptional control of the early/late vaccinia p7.5 promoter). More preferably, the encoded MUC1 antigen comprises an amino acid sequence that is at least 90% identical to SEQ ID NO: 12. In another preferred embodiment, the therapeutic vaccine for use in the invention comprises an Ad vector which contains inserted into its genome (preferably in region E1) a nucleic acid molecule encoding a fusion of HBV antigens including HBc (e.g. a C-term truncated version of core, a pol antigen disrupted for polymerase and RNAse H enzymatic activities and two env immunogenic domains, preferably under the transcriptional control of the CMV promoter, with a specific preference for an HBV antigen fusion comprising an amino acid sequence that is at least 80% identical to SEQ ID NO: 17 or SEQ ID NO: 18 (corresponding to SEQ ID NO: 8 and 12 of WO2013/007772) or encoded by a nucleotide sequence comprising a sequence at least 80% identical to SEQ ID NO: 15 of WO2013/007772.

### Production of Virus-Based Therapeutic Vaccine

In a preferred embodiment, the therapeutic vaccine comprised in the first composition for use according to the present invention is a viral vector. Typically, viral vectors are produced into suitable host cells using conventional techniques including a) preparing a producer (e.g. permissive) host cell, b) transfecting or infecting the prepared producer host cells, c) culturing the transfected or infected host cell under suitable conditions so as to allow the production of the vector (e.g. infectious viral particles), d) recovering the produced vector from the culture of said cell and optionally e) purifying said recovered vector.

In step a), suitable producer cells depend on the type of viral vector to be amplified. Replication-defective recombinant adenoviruses are typically propagated and produced in a cell that supplies in trans the adenoviral protein(s) encoded by those genes that have been deleted or inactivated in the replication-defective adenovirus, thus allowing the virus to replicate in the cell. Suitable cell lines for complementing E1-deleted adenoviruses include the HEK-293 cells (Graham et al., 1997, J. Gen. Virol. 36: 59-72), HER-96 and PER-C6 cells (e.g. Fallaux et al., 1998, Human Gene Ther. 9: 1909-1917; WO97/00326) or any derivative of these cell lines. But any other cell line described in the art can also be used in the context of the present invention, especially cell lines approved for producing products for human use. The infectious adenoviral particles may be recovered from the culture supernatant and/or from the cells after lysis. They can be further purified according to standard techniques (ultracentrifugation in a cesium chloride gradient, chromatography, etc. as described for example in WO96/27677, WO98/00524, WO98/22588, WO98/26048, WO00/40702, EP1016711 and WO00/50573).

MVA is strictly host-restricted and is typically amplified on avian cells, either primary avian cells (such as chicken embryo fibroblasts (CEF) prepared from chicken embryos obtained from fertilized eggs) or immortalized avian cell lines. Representative examples of suitable avian cell lines for MVA production include without limitation the Cairina moschata cell lines immortalized with a duck TERT gene (see e.g. WO2007/077256, WO2009/004016, WO2010/130756 and WO2012/001075); avian cell line immortalized with a combination of viral and/or cellular genes (see e.g. WO2005/042728); a spontaneously immortalized cell (e.g. the chicken DF1 cell line disclosed in U.S. Pat. No. 5,879,924); or immortalized cells which derive from embryonic cells by progressive severance from growth factors and feeder layer (e.g. Ebx chicken cell lines disclosed in WO2005/007840 and WO2008/129058).

For other vaccinia virus or other poxvirus strains, in addition to avian primary cells (such as CEF) and avian cell lines, many other non-avian cell lines are available for production, including human cell lines such as HeLa (ATCC-CRM-CCL-2™ or ATCC-CCL-2.2™), MRC-5, HEK-293; hamster cell lines such as BHK-21 (ATCC CCL-10), and Vero cells. In a preferred embodiment, non-MVA vaccinia virus are amplified in HeLa cells (see e.g. WO2010/130753).

Producer cells are preferably cultivated in a medium free of animal-or human-derived products, using a chemically defined medium with no product of animal or human origin. In particular, while growth factors may be present, they are preferably recombinantly produced and not purified from animal material. An appropriate animal-free medium may be easily selected by those skilled in the art depending on selected producer cells. Such media are commercially available. In particular, when CEFs are used as producer cells, they may be cultivated in VP-SFM cell culture medium (Invitrogen). Producer cells are preferably cultivated at a temperature comprised between +30° C. and +38° C. (more preferably at about +37° C.) for between 1 and 8 days (preferably for 1 to 5 days for CEF and 2 to 7 days for immortalized cells) before infection. If needed, several passages of 1 to 8 days may be made in order to increase the total number of cells.

In step b), producer cells are infected by the viral vector under appropriate conditions (in particular using an appropriate multiplicity of infection (MOI) to permit productive infection of producer cells. In particular, when the therapeutic vaccine is based on MVA and is amplified using CEF, it may be seeded in the cell culture vessel containing CEFs at a MOI which is preferably comprised between 0.001 and 1 (more preferably about 0.05). Adenovirus vectors are preferably used at MOI comprised between 0.1 and 100. Infection step is also preferably performed in a medium (which may be the same as or different from the medium used for culture of producer cells) free from animal- or human-derived products, using a chemically defined medium with no product of animal or human origin.

In step c), infected producer cells are then cultured under appropriate conditions well known to those skilled in the art until progeny viral vector (e.g. infectious virus particles) is produced. Culture of infected producer cells is also preferably performed in a medium (which may be the same as or different from the medium used for culture of producer cells and/or for infection step) free of animal- or human-derived products (using a chemically defined medium with no product of animal or human origin) at a temperature between +30° C. and +37° C., for 1 to 5 days.

In step d), the viral vector produced in step c) is collected from the culture supernatant and/or the producer cells. Recovery from producer cells (and optionally also from culture supernatant), may require a step allowing the disruption of the producer cell membrane to allow the liberation of the vector from producer cells. The disruption of the producer cell membrane can be induced by various techniques well known to those skilled in the art, including but not limited to: freeze/thaw, hypotonic lysis, sonication, microfluidization, or high speed homogenization.

Viral vectors may then be further purified, using purification steps well known in the art. Various purification steps can be envisaged, including clarification, enzymatic treatment (e.g. endonuclease, protease, etc.), chromatographic and filtration steps. Appropriate methods are described in the art (e.g. WO2007/147528; WO2008/138533, WO2009/100521, WO2010/130753, WO2013/022764).

### TLR9 Ligand Oligonucleotide—Li28

In one embodiment, the oligonucleotide comprised in the second composition of the invention is a synthetic single-stranded oligodeoxynucleotide containing at least 3 unmethylated CpG motifs which is capable of binding a mammal TLR9 receptor (TLR9 ligand).

The number of nucleotide residues comprised in the oligonucleotide in use herein is not critical, and oligonucleotides having from 21 nucleotide residues to approximately 100 nucleotide residues are more specifically contemplated in the present invention. A preferred oligonucleotide comprises from 21 to 60 nucleotides, advantageously from 22 to 50 nucleotides, desirably from 23 to 40 nucleotides, preferably from 24 to 35 nucleotides, more preferably from 25 to 30 nucleotides and even more preferably 26, 27, 28, 29 or 30 nucleotides with an absolute preference for a 26 mer (i.e. 26 nucleotides long oligonucleotide).

In a preferred embodiment, the oligonucleotide in use in this invention is stabilized against in vivo degradation using chemical means (e.g. modification of the oligonucleotide backbone) or protection by suitable compounds (e.g. polymers, lipids, synthetic compounds). In particular, instead of having a phosphodiester (PO) backbone (as found in genomic bacterial DNA) which is known to be more sensitive to the nucleases present in human cells, the oligonucleotide in use herein possesses a partially or completely chemically stabilized backbone such as a phosphodiester, phosphorothioate (PS), methylphosphonated or phosphorodithioate backbone or combinations of such linkages. Preferably, the oligonucleotide in use in the present invention comprises a phosphorothioated backbone. Alternatively or in combination, the oligonucleotide can also be stabilized by inclusion in a colloidal suspension, such as liposomes, polymers, solid lipid particles, or polyalkylcyanoacrylate nanoparticles (Muller, 2000, Eur. J. Pharm. Biopharm. 50: 167-77; Lambert et al., 2001, Adv. Drug

Deliv. Rev., 47, 99-112; Delie et al., 2001 Int. J. Pharm. 214, 25-30).

The number of unmethylated CpG motifs comprised in the oligonucleotide for use herein is not limited. In one embodiment, it contains from 3 to 20 CpG motifs, from 3 to 19 CpG motifs, from 3 to 18 CpG motifs, from 3 to 17 CpG motifs, from 3 to 16 CpG motifs, from 3 to 15 CpG motifs, from 3 to 14 CpG motifs, from 3 to 13 CpG motifs, from 3 to 12 CpG motifs, from 3 to 11 CpG motifs, from 3 to 10 CpG motifs, from 3 to 9 CpG motifs, from 3 to 8 CpG motifs, from 3 to 7 CpG motifs, from 3 to 6 CpG motifs, from 3 to 5 CpG motifs, 3 or 4 CpG motifs, with a preference for 3 CG motifs.

In one embodiment, the at least 3 CpG motifs comprised in the oligonucleotide in use herein are in a particular sequence context which independently may be represented as the following 6 mer motif:

5′-RRCGYY-3′ (“purine-purine-C-G-pyrimidine-pyrimidine”, SEQ ID NO:13) or 5′-RYCGYY-3′ (“purine-pyrimidine-C-G-pyrimidine-pyrimidine”, SEQ ID NO:14) wherein each R occurrence is a purine nucleotide or a purine nucleotide derivative (i.e. A or G, wherein A is an adenosine nucleotide or an adenosine nucleotide derivative and G is a guanosine nucleotide or a guanosine nucleotide derivative); C is a cytosine nucleotide or a cytosine nucleotide derivative; G is a guanosine nucleotide or a guanosine nucleotide derivative; Y is a pyrimidine nucleotide or a pyrimidine nucleotide derivative (C or T wherein C is as above and T is a thymidine nucleotide or a thymidine nucleotide derivative). Desirably, at least one of said hexameric motifs is palindromic. In a particular embodiment, at least one of the bases of the hexameric motif described above can be modified, in particular, at least one of the cytosines can be replaced with a 5-bromocytosine.

In one embodiment, the oligonucleotide comprises a nucleotide sequence as shown in SEQ ID NO: 1 (RN₃CGYY) with N₃ being a purine (A or G) or a pyrimidine (C or T) nucleotide or a nucleotide derivative thereof, and optionally one or two additional nucleotides in 5′ (N₁N₂) and/or one or two additional nucleotides in 3′ (N₄N₅), with each of N₁, N₂, N₄, and N₅ being a purine (A or G) or a pyrimidine (C or T) nucleotide or a nucleotide derivative thereof. In this case, the oligonucleotide comprises one of the nucleotide sequences shown in:
SEQ ID NO: 1 (RN₃CGYY), with N₃ being a purine (A or G) or a pyrimidine (C or T) nucleotide or a nucleotide derivative thereof,
SEQ ID NO:2 (N₂RN₃CGYY), with each of N₂ and N₃ being independently a purine (A or G) or a pyrimidine (C or T) nucleotide or a nucleotide derivative thereof,
SEQ ID NO:3 (N₁N₂RN₃CGYY), with each of N₁, N₂ and N₃ being independently a purine (A or G) or a pyrimidine (C or T) nucleotide or a nucleotide derivative thereof,
SEQ ID NO:4 (RN₃CGYYN₄), with each of N₃ and N₄ being independently a purine (A or G) or a pyrimidine (C or T) nucleotide or a nucleotide derivative thereof,
SEQ ID NO:5 (RN₃CGYYN₄N₅), with each of N₃, N₄ and N₅ being independently a purine (A or G) or a pyrimidine (C or T) nucleotide or a nucleotide derivative thereof,
SEQ ID NO:6 (N₂RN₃CGYYN₄), with each of N₂, N₃ and N₄ being independently a purine (A or G) or a pyrimidine (C or T) nucleotide or a nucleotide derivative thereof,
SEQ ID NO:7 (N₂RN₃CGYYN₄N₅), with each of N₂, N₃, N₄ and N₅ being independently a purine (A or G) or a pyrimidine (C or T) nucleotide or a nucleotide derivative thereof,
SEQ ID NO:8 (N₁N₂RN₃CGYYN₄), with each of N₁, N₂, N₃ and N₄ being independently a purine (A or G) or a pyrimidine (C or T) nucleotide or a nucleotide derivative thereof, and
SEQ ID NO:9 (N₁N₂RN₃CGYYN₄N₅), with each of N₁, N₂, N₃, N₄ and N₅ being independently a purine (A or G) or a pyrimidine (C or T) nucleotide or a nucleotide derivative thereof.

In a preferred embodiment, the at least 3 hexameric motifs represented as RRCGYY (SEQ ID NO:13) are preferably AACGTT (SEQ ID NO:15) and those represented as RYCGYY (SEQ ID NO:14) are preferably GTCGTT (SEQ ID NO:16).

According to an advantageous arrangement of this embodiment, the at least 3 hexameric motifs comprised in the oligonucleotide for use herein may independently be adjacent (i.e. 0 nucleotide in between) or may have intervening nucleotides located between two motifs. In accordance with the “separated” embodiment, the number of intervening nucleotides between two hexameric motifs may independently varies from 1 to 20 nucleotides (e.g. 1, 2, 3, 4, 5, 6, 7, 8, 9, 10, 11, 12, 13, 14, 15, 16, 17, 18, 19 or 20 nucleotides). A preferred embodiment is directed to 2 nucleotides in between each hexameric motif (preferably AT or TT). On the same line, there may be some (e.g. 2) nucleotides in 5′ of the first hexameric motif and/or some nucleotides in 3′ of the last one with a specific preference for TA or TC before the first hexameric motif and either no nucleotide or AT following the last hexameric motif present in the oligonucleotide.

A preferred embodiment is directed to Litenimod (Li28 or CpG-28) described by Carpentier et al. (Carpentier et al., 2003, Frontiers in Bioscience 8, e115-127; Carpentier et al., 2006, Neuro-Oncology 8(1): 60-6; EP 1 162 982; U.S. Pat. Nos. 7,700,569 and 7,108,844) or derivative thereof (e.g. at least 85% identity and preferably at least 90% identity). A preferred oligonucleotide for use in the combination of the present invention comprises, essentially consists of, consists of a nucleotide sequence as shown in SEQ ID NO: 10 (5′-TAAACGTTATAACGTTATGACGTCAT-3′). Another suitable oligonucleotide comprises, essentially consists of, consists of a nucleotide sequence as shown in SEQ ID NO: 11 (5′-TCGTCGTTTTGTCGTTTTGTCGTT-3′)

The present invention encompasses an immunostimulatory combination comprising one or more type(s) of CpG oligonucleotide. In a particular embodiment, the one or more oligonucleotide(s) for use in this invention can be encoded by the therapeutic vaccine described herein. For example, a double stranded linear oligonucleotide can be generated by chemical synthesis and one or more copy can be inserted in a vector-based therapeutic vaccine (e.g. in an antigen-encoding viral vector). The oligonucleotide and the nucleic acid molecule(s) encoding the polypeptide(s) of interest can be expressed independently using distinct regulatory elements or, alternatively, from an independent vector system such as one of those described herein in connection with the therapeutic vaccine for separate or concomitant administration to the subject in need thereof. Such an embodiment is especially appropriate for non-cytoplasmic vectors such as adenoviruses.

In certain embodiments, the oligonucleotides can advantageously be coupled, via covalent, ionic or weak attachments, to a molecule or a group of molecules which modify its activity, its affinity, its detection and/or its delivery, such as, among other possibilities, detectable labels, cytotoxic compounds, targeting compounds and/or delivery means. Detectable labels can facilitate detection of the oligonucleotide or the immunostimulatory combination within a host cell or a subject. Detection can be made through radioactive, fluorescent or enzymatic compounds, etc. Radioactive isotopes may be used to make the oligonucleotide detectable by radioactive detection means or makes cells comprising the radiolabeled oligonucleotide more sensitive to radiation therapy. Suitable radioactive compounds include, but are not limited to, metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuhdine (IUdR), bromodeoxycytidine, fluorodeoxyuridine (FUdR), hydroxyurea and cisplatin. Generally, fluorescent labels use photochromic compounds having the ability to display different colors according to their absorbance in different wavelengths of light. Enzymatic labels are able to catalyze chemical modification of a substrate compound which becomes detectable. “Cytotoxic compounds” may be directly toxic to cells, preventing their reproduction or growth such as toxins (e. g. an enzymatically active toxin of bacterial, fungal, plant or animal origin, or fragments thereof). Targeting can confer specific binding to a particular target and allow for uptake in a cell bearing said target. Targeting may be performed through complexation to peptides, antibodies or fragments thereof for targeting specific cells (e.g. cells expressing a tumor antigen) cell types (e.g. hepatic cells) or specific molecules (e.g. receptors on the surface of tumor cells).

In certain embodiments, the oligonucleotides disclosed herein can be delivered to the subject upon association with liposomes, nanoparticles, etc. (e.g. U.S. Pat. No. 8,680,045).

### Combination Therapy

The term “combination” as used herein refers to any arrangement possible of at least the two entities that are subject of the present invention (i.e. the first composition comprising the therapeutic vaccine and the second composition comprising the oligonucleotide described herein). Preferably, the combination is synergistic providing higher efficacy (e.g. improved immune response, survival, antiviral effect, etc.) than each entity alone. “Combination therapy” and any variation such as “combined use” refers to the action of delivering to the same subject such entities.

In one embodiment, the first and second compositions may be placed together in a common container before being administered to the subject.

In another embodiment, the first and the second compositions are not mixed together meaning that they are into separate containers (individual entities) for administration to the subject in conjunction with one another, either concomitantly, sequentially or in an interspersed manner.

Exemplary immunostimulatory combinations include, but are not limited to, combination of polypeptide-based therapeutic vaccine (e.g. in the form of recombinant protein or adjuvanted peptides) or nucleic acid-based therapeutic vaccine (e.g. a vectorized therapeutic vaccine) with one or more oligonucleotide(s) described herein such as Litenimod. The present invention encompasses combinations comprising equal molar concentrations of each entity as well as combinations with very different concentrations of the different entities. It is appreciated that optimal concentration of each entity can be determined by the artisan skilled in the art.

### Compositions

In one embodiment, the first composition comprises a therapeutically or immunologically effective amount of a therapeutic vaccine described herein and the second composition comprises a therapeutically or immunologically effective amount of a one or more oligonucleotide(s) described herein. Such a therapeutically or immunologically effective amount may vary as a function of various parameters such as the composition itself (kind of therapeutic vaccine and oligonucleotide), the disease to be treated (e.g. nature and severity of symptoms, kind of concurrent treatment, the need for prevention or therapy, etc.), the subject (age, weight, its ability to respond to the treatment), and/or the mode of administration; etc.

The preparation of compositions is well known in the art. In one embodiment, each of the first (therapeutic vaccine) and the second (oligonucleotide) compositions may comprise a pharmaceutically acceptable vehicle which can be the same or different. The term “pharmaceutically acceptable vehicle” is intended to include any and all carriers, solvents, diluents, excipients, adjuvants, dispersion media, coatings, antibacterial and antifungal agents, absorption agents and the like compatible for human use.

Various formulations can be envisaged in the context of the invention for each of the first and second compositions, either liquid or freeze-dried form to ensure stability under the conditions of manufacture and long-term storage (i.e. for at least 6 months) at freezing (e.g. −70° C., −20° C.), refrigerated (e.g. 4° C.) or ambient (e.g. 20-25° C.) temperature.

Liquid compositions generally include a liquid vehicle such as physiological saline solution, Ringer's solution, Hank's solution, saccharide solution (e.g. glucose, trehalose, saccharose, dextrose, etc.) and other aqueous physiologically balanced salt solutions (see for example the most current edition of Remington: The Science and Practice of Pharmacy, A. Gennaro, Lippincott, Williams&Wilkins). Animal or vegetable oils, mineral or synthetic oils are also suitable.

In one embodiment, the first composition (therapeutic vaccine) is preferably formulated for storage at freezing or refrigerated temperature and the second composition (oligonucleotide) is formulated in lyophilized form that is then diluted in physiological saline (0.9% of sodium chloride) before use.

If needed, the first and/or second composition(s) may also include a cryoprotectant so as to protect the therapeutic vaccine and/or the one or more oligonucleotide(s) at low storage temperature. Suitable cryoprotectants include without limitation sucrose (or saccharose), trehalose, maltose, lactose, mannitol, sorbitol and glycerol, preferably in a concentration of 0.5 to 20% (weight in g/volume in L, referred to as w/v). For example, sucrose is preferably present in a concentration of 5 to 15% (w/v), with a specific preference for about 10%. The presence of high molecular weight polymers such as dextran or polyvinylpyrrolidone (PVP) is particularly suited for lyophilized formulations to protect the biological product during the vacuum drying and freeze-drying steps (see e.g. WO03/053463; WO2006/0850082; WO2007/056847; WO2008/114021) and the presence of these polymers assists in the formation of the cake during freeze-drying (see EP1418942 and WO2014/053571).

The composition(s) (especially liquid compositions) may further comprise a pharmaceutically acceptable chelating agent, and in particular an agent chelating dications for improving stability. The pharmaceutically acceptable chelating agent may notably be selected from ethylenediaminetetraacetic acid (EDTA), 1,2-bis(o-aminophenoxy)ethane-N,N,N′,N′-tetraacetic acid (BAPTA), ethylene glycol tetraacetic acid (EGTA), dimercaptosuccinic acid (DMSA), diethylene triamine pentaacetic acid (DTPA), and 2,3-Dimercapto-1-propanesulfonic acid (DMPS). The pharmaceutically acceptable chelating agent is preferably present in a concentration of at least 50 μM with a specific preference for a concentration of 50 to 1000 μM. Preferably, said pharmaceutically acceptable chelating agent is EDTA present in a concentration close to 150 μM.

It might also be beneficial to also include a monovalent salt so as to ensure an appropriate osmotic pressure. Said monovalent salt may notably be selected from NaCl and KCl, preferably said monovalent salt is NaCl, preferably in a concentration of 10 to 500 mM.

In one embodiment, the first and/or the second compositions can be suitably buffered, preferably at physiological or slightly basic pH (e.g. from approximately pH 7 to approximately pH 9 with a specific preference for a pH comprised between 7 and 8 and more particularly close to 7.5) for human use. Suitable buffers include without limitation TRIS (tris(hydroxymethyl)methylamine), TRIS-HCl (tris(hydroxymethyl)methylamine-HCl), HEPES (4-2-hydroxyethyl-1-piperazineethanesulfonic acid), phosphate buffer (e.g. PBS), ACES (N-(2-Acetamido)-aminoethanesulfonic acid), PIPES (Piperazine-N,N′-bis(2-ethanesulfonic acid)), MOPSO (3-(N-Morpholino)-2-hydroxypropanesulfonic acid), MOPS (3-(N-morpholino)propanesulfonic acid), TES (2-{[tris(hydroxymethyl)methyl]amino}ethanesulfonic acid), DIPSO (3-[bis(2-hydroxyethyl)amino]-2-hydroxypropane-1-sulfonic acid), MOBS (4-(N-morpholino)butanesulfonic acid), TAPSO (3-[N-Tris(hydroxymethypmethylamino]-2-hydroxypropanesulfonic Acid), HEPPSO (4-(2-Hydroxyethyl)-piperazine-1-(2-hydroxy)-propanesulfonic acid), POPSO (2-hydroxy-3-[4-(2-hydroxy-3-sulfopropyl)piperazin-1-yl]propane-1-sulfonic acid), TEA (triethanolamine), EPPS (N-(2-Hydroxyethyl)-piperazine-N′-3-propanesulfonic acid), and TRICINE (N-[Tris(hydroxymethyl)-methyl]-glycine). TRIS-HCl, TRIS, Tricine, HEPES and phosphate buffer comprising a mixture of Na₂HPO₄ and KH₂PO₄ or a mixture of Na₂HPO₄ and NaH₂PO₄ are preferred in the context of the invention. For illustrative purposes, a buffer concentration of 10 to 50 mM (in particular for TRIS-HCl) is appropriate.

Additional compounds may further be present to increase stability of the formulated therapeutic vaccine and/or oligonucleotide composition(s). Such additional compounds include, without limitation, C₂-C₃ alcohol (desirably in a concentration of 0.05 to 5% (volume/volume or v/v)), sodium glutamate (desirably in a concentration lower than 10 mM), non-ionic surfactant (Evans et al. 2004, J Pharm Sci. 93:2458-75, Shi et al., 2005, J Pharm Sci. 94:1538-51, U.S. Pat. No. 7,456,009, US2007/0161085) such as Tween 80 (also known as polysorbate 80) at low concentration below 0.1%. Divalent salts such as MgCl₂ or CaCl₂ have been found to induce stabilization of various biological products in the liquid state (see Evans et al. 2004, J Pharm Sci. 93:2458-75 and U.S. Pat. No. 7,456,009). Amino acids, and in particular histidine, arginine or methionine, have been found to induce stabilization of various viruses in the liquid state (see Evans et al., 2004, J Pharm Sci. 93:2458-75, U.S. Pat. No. 7,456,009, US2007/0161085, U.S. Pat. No. 7,914,979, WO2014/029702 and WO2014/053571).

In one embodiment, the first and/or the second compositions may be adjuvanted to further enhance immunity. Representative examples of suitable adjuvants include, without limitation, alum, mineral oil emulsion such as, Freunds complete and incomplete (IFA), lipopolysaccharides (Ribi et al., 1986, Immunology and Immunopharmacology of Bacterial Endotoxins, Plenum Publ. Corp., NY, p407-419), saponins such as ISCOMATRIX, AbISCO, QS21 (Sumino et al., 1998, J. Virol. 72: 4931; WO98/56415), imidazo-quinoline compounds such as Imiquimod (Suader, 2000, J. Am Acad Dermatol. 43: S6), S-27609 (Smorlesi, 2005, Gene Ther. 12: 1324) and related compounds such as those described in WO2007/147529; cationic peptides such as IC-31 (Kritsch et al., 2005, J. Chromatogr Anal. Technol. Biomed. Life Sci. 822: 263-70), polysaccharides such as Adjuvax, squalenes such as MF59 and RIG-I-like agonists such as SB9200.

The formulation of the first and/or second compositions can also be adapted to the mode of administration to ensure proper distribution or delayed release in vivo. For example, gastro-resistant capsules and granules are particularly appropriate for oral administration, suppositories for rectal or vaginal administration, eventually in combination with absorption enhancers useful to increase the pore size of the mucosal membranes. Such absorption enhancers are typically substances having structural similarities to the phospholipid domains of the mucosal membranes (such as sodium deoxycholate, sodium glycocholate, dimethyl-beta-cyclodextrin, lauryl-1-lysophosphatidylcholine).

### Administration and Doses

Any of the conventional administration routes is applicable in the context of the invention including parenteral, topical or mucosal routes. Parenteral routes are intended for administration as an injection or infusion and encompass systemic as well as local routes and include without limitation intravenous (into a vein), intravascular (into a blood vessel), intra-arterial (into an artery), intradermal (into the dermis), transcutaneous, subcutaneous (under the skin), intramuscular (into muscle), intraperitoneal (into the peritoneum), intracerebral (into the brain), intranodal (e.g. into a lymph node) and intratumoral (into a tumor or its close vicinity) routes as well as scarification. Infusions typically are given by intravenous route or intratumoral (in a large tumor). Mucosal administrations include without limitation oral/alimentary, intranasal, intratracheal, nasopharyngeal, intrapulmonary, intravaginal or intra-rectal route. Although administration routes may vary for delivering each of the first and second compositions, preferred routes of administration for both of them include intravenous, intramuscular, subcutaneous and intratumoral. More specifically, the therapeutic vaccine and the oligonucleotide compositions are preferably administered by subcutaneous, intramuscular, intraperitoneal, intravenous or intratumoral injections either at the same site, at close proximity or at different sites allowing the target of the infected organ and the priming in periphery of T cells. Of course, the routes of administration for each of the first and second compositions can be adapted to the therapeutic vaccine, the oligonucleotide composition and the targeted indication. For illustrative but non limitating purposes, an oncolytic virus-based therapeutic vaccine can be injected intravenously or intratumorally as the oligonucleotide composition whereas a MVA-based composition is preferably administered by subcutaneous or transcutaneous route. On the other hand, a therapeutic vaccine targeting an infectious disease such as HBV (e.g. the AdTG18201 illustrated in the Example section) will preferably be injected by subcutaneous or intramuscular route to prime immune cells whereas the oligonucleotide composition can be injected by a route suitable to reach the site of infection (e.g. intravenously to target the liver).

Administrations may use standard needles and syringes or any device available in the art capable of facilitating or improving delivery including for example catheters, electric syringe, Quadrafuse injection needles, needle-free injection devices (e.g. Biojector TM device), infusion pumps etc. Electroporation may also be implemented to facilitate intramuscular administration. Topical administration can also be performed using transdermal means (e.g. patch and the like). Systems are being developed using solid, hollow, coated or dissolvable microneedles (see e.g., Van der Maaden et al., 2012, J. Control release 161: 645-55) and preferred are silicon and sucrose microneedle patches (see, e.g., Carrey et al., 2014, Sci Rep 4: 6154 doi 10.1038; and Carrey et al., 2011, PLoS ONE, 6(7) e22442).

The actual amount of the first and the second compositions to administer to the subject may be routinely made by a practitioner in the light of the relevant circumstances (age, body weight, symptoms, clinical state, route of administration, duration of the treatment, etc. as mentioned above) Further refinement of the calculations can be necessary to adapt the appropriate dosage for a subject or a group of subjects.

For illustrative purposes, suitable dosage of the second composition especially for parenteral administration varies from about 1 μg to 200 mg, advantageously from about 0.01 mg to about 100 mg, desirably from about 0.05 mg to about 50 mg, preferably from about 0.1 mg to about 40 mg, more preferably from about 0.25 mg to about 25 mg, and more specifically from about 0.5 mg to about 20 mg, with a specific preference for doses of 0.5 mg, 1 mg, 2 mg, 5 mg, 10 mg or 15 mg. However, lower doses may be envisaged for localized administration.

Suitable dosage for a virus-based first composition varies from approximately 10⁴ to approximately 10¹³ vp (viral particles), iu (infectious unit) or pfu (plaque-forming units) of a viral vector depending on the viral vector and quantitative technique used. As a general guidance, adenovirus doses from approximately 10⁵ to approximately 5×10¹² vp are suitable, preferably from approximately 10⁶ vp to approximately 10¹² vp, more preferably from approximately 10⁷ vp to approximately 5×10¹¹ vp; doses of approximately 10⁸ vp to approximately 10¹¹ vp being particularly preferred especially for parenteral delivery. Individual doses which are suitable for vaccinia virus-based therapeutic vaccine comprise from approximately 10⁴ to approximately 10¹³ pfu. More specifically, suitable doses of replication-defective vaccinia-based composition such as MVA comprises from approximately 10⁴ to approximately 10¹² pfu, preferably from approximately 10⁵ pfu to approximately 10¹¹ pfu, more preferably from approximately 10⁶ pfu to approximately 10¹⁰ pfu;

doses of approximately 10⁷ pfu to approximately 10⁹ pfu being particularly preferred especially for human use. Individual doses which are suitable for oncolytic Vaccinia-based therapeutic vaccine comprise from approximately 10⁵ to approximately 10¹³ pfu, preferably from approximately 10⁶ pfu to approximately 10¹¹ pfu, more preferably from approximately 10⁷ pfu to approximately 10¹⁰ pfu; doses of approximately 10⁸ pfu to approximately 5×10⁹ pfu being particularly preferred especially for human use. The quantity of virus present in a sample can be determined by routine titration techniques, e.g. by counting the number of plaques following infection of permissive cells (e.g. 293 or PERC6 or HER96 for Ad, BHK-21 or CEF for MVA, HeLa for VV), by measuring the A260 absorbance (vp titers), or still by quantitative immunofluorescence, e.g. using anti-virus antibodies (iu titers). Suitable dosage for a plasmid-based therapeutic vaccine varies from 10 μg to 20 mg, advantageously from 100 μg to 10 mg and preferably from approximately 0.5 mg to approximately 5 mg.

### Time Course Administration

The immunostimulatory combination of the invention is suitable for a single administration or a series of administrations which can be concomitant (e.g. mixture of first and second compositions or administration of the first and second compositions at approximately the same time), sequential (in either order) or interspersed (intermixed administrations at various time intervals). Moreover, the various administrations may be performed by the same or different routes at the same site or at alternative sites with the same or different dosages and the sequence of the multiple administrations and intervals in between may vary. The doses can vary for each administration within the range described above. Intervals between the various administrations (e.g. between the therapeutic vaccine administrations, between the oligonucleotide administrations and/or between the therapeutic vaccine and oligonucleotide administrations can be regular or irregular (e.g. dependent on measurements specific to the targeted disease). One may also proceed via sequential cycles of administrations that are repeated after a rest period.

In one embodiment, the first and the second compositions are administered sequentially, with a specific preference for the administration of therapeutic vaccine being initiated before the administration of the oligonucleotide. “Sequential” as used herein means a time interval of at least one hour to approximately a week between at least one administration of the therapeutic vaccine and one administration of the oligonucleotide. Advantageously, such time interval is from approximately 2 hours to approximately 4 days, preferably from approximately 6 hours to approximately 3 days and even more preferably from approximately 6 hours to approximately 48 hours (e.g. 6, 7, 8, 9, 10, 12, 14, 18, 20, 24, 28, 32, 36, 40, 44 or 48 h) with a specific preference for about 24 hours.

In a preferred embodiment, the immunostimulatory combination of the present invention is administered to the subject at least twice (e.g. 2, 3, 4, 5, 6, 7, 8, 9, 10, 11, 12, 13, 14, 15, 16, etc) and, preferably, comprises from 2 to 10 sequential administrations of the first and the second compositions. More preferably, the at least twice injections of the first composition (therapeutic vaccine) is followed 6 h to 48 h later by an injection of the second composition (oligonucleotide), preferably at the same site or at its close proximity or at a site around a site of infection.

In one exemplary regimen, the subject received 2 to 10 administrations of the first composition followed by 2 to 10 administrations of the second composition at a 6 to 48 h interval (e.g. 24 h). For example, a MVA-based composition is administered 2 to 10 times (e.g. subcutaneously or intratumoral) at weekly intervals at a dose of about 10⁷ to 10⁹ pfu, and each MVA injection is followed 24 h later by an injection (e.g. subcutaneous or intratumoral) of the oligonucleotide composition at the same site or at its close proximity. Several cycles of such administration regimen can be envisaged after a rest period (e.g. 1 week to 6 months). In one embodiment, the first composition comprises a MVA encoding MUC-1 (and optionally IL-2) and the second composition comprises litenimod.

However, the present invention also encompasses other regimens as long as the immunostimulatory combination comprises at least one administration of the first composition followed by (e.g. 6 h-48 h later) one administration of the second composition. An exemplary regimen may include further administrations of the first and/or second composition carried out before and/or after the sequential administration(s) of the first and the second compositions. For illustrative purpose, a suitable regimen comprises 3 weekly administrations (D0, D7 and D14) of about 10⁷ to 5×10¹¹ vp of an Ad-based composition, and 3 weekly administrations (D9, D16 and D23) of the oligonucleotide composition, in order that the two sequential administrations of the Ad vector and the CpG oligonucleotides (at 48 h intervals) are preceded by one administration of the therapeutic vaccine (D0) and followed by one administration of the oligonucleotide (D23). In one embodiment, the first composition comprises an adenovirus encoding HBV antigens (e.g. as described in WO2013/007772) and the second composition comprises litenimod.

### Therapeutic Indications

In the context of the invention, the immunostimulatory combination of the present invention can be used as a medicament for prophylaxis (e.g. to reduce the risk of having a given disease or pathological condition) and/or therapy (e.g. in a subject diagnosed as having a given disease or pathological condition). When “prophylactic” use is concerned, the immunostimulatory combination is administered at a dose sufficient to prevent or to delay the onset and/or establishment and/or relapse of a pathologic condition, especially in a subject at risk. For “therapeutic” use, the first and second compositions are both administered to a subject diagnosed as having a disease or pathological condition with the goal of treating it, eventually in association with one or more conventional therapeutic modalities. Therapeutic use is preferred in the context of the present invention.

Because of its ability to enhance immune response, the immunostimulatory combination of the invention is/are particularly useful as a medicament, especially for treating or preventing diseases or pathologic condition, such as proliferative diseases involving abnormal proliferation of cells (e.g. cancer) and infectious diseases (e.g. chronic viral infections). Such diseases (and any form of disease such as “disorder” or “pathological condition”) are typically characterized by identifiable symptoms. Administration of the immunostimulatory combination of the invention can be carried out at dosages and for periods of time effective to reduce symptoms or surrogate markers of the disease.

As used herein, the term “proliferative disease” encompasses any disease or condition resulting from uncontrolled cell growth and spread including cancers as well as diseases associated to an increased osteoclast activity (e.g. rheumatoid arthritis, osteoporosis, etc.) and cardiovascular diseases (restenosis that results from the proliferation of the smooth muscle cells of the blood vessel wall, etc). The term “cancer” may be used interchangeably with any of the terms “tumor”, “malignancy”, “neoplasm”, etc. These terms are meant to include any type of tissue, organ or cell, any stage of malignancy (e.g. from a prelesion to stage IV) encompassing solid tumors and blood borne tumors and primary and metastatic cancers whatever their nature and their degree of anaplasia. Representative examples of cancers that may be treated using the immunostimulatory combination and methods of the invention include, without limitation, carcinoma, lymphoma, blastoma, sarcoma, and leukemia and more particularly bone cancer, gastrointestinal cancer, liver cancer, pancreatic cancer, gastric cancer, colorectal cancer, esophageal cancer, oro-pharyngeal cancer, laryngeal cancer, salivary gland carcinoma, thyroid cancer, lung cancer, cancer of the head or neck, skin cancer, squamous cell cancer, melanoma, uterine cancer, cervical cancer, endometrial carcinoma, vulvar cancer, ovarian cancer, breast cancer, prostate cancer, cancer of the endocrine system, sarcoma of soft tissue, bladder cancer, renal cancer, kidney cancer and cancers of the central and peripheral nervous systems, including astrocytomas, glioblastomas, medulloblastomas and neuroblastomas. The present invention is particularly useful for the treatment of renal cancer (e.g. clear cell carcinoma), bladder cancer, prostate cancer (e.g. hormone refractory prostate adenocarcinoma), breast cancer (e.g. metastatic breast cancer), colorectal cancer, lung cancer (e.g. non-small cell lung cancer), liver cancer (e.g. hepatocarcinoma), gastric cancer, pancreatic cancer, melanoma, ovarian cancer and glioblastoma, and especially metastatic ones. In certain embodiments, a combination comprising a MUC-1 encoding vector (e.g. TG4010) and an oligonucleotide such as Li28 is particularly appropriate for the treatment of cancers that overexpress MUC-1 (especially hypoglycosylated form thereof) such as renal, lung and breast cancers.

As used herein, infectious diseases result from an infection with a pathogenic organism (e.g. bacteria, parasite, virus, fungus, etc.). It may be particularly useful for treating HBV infection, especially a chronic one, relying on the administration of (a) a therapeutic vaccine comprising a vector (e.g. an adenovirus) encoding HBV antigen(s) and (b) one or more CpG oligonucleotide(s) in an amount sufficient to treat or prevent in a subject in need thereof or alleviate one or more symptoms related to HBV-associated diseases and pathologic conditions, according to the modalities described herein. In certain embodiments, a combination comprising a vector encoding HBV antigens (e.g. TG1050) and an oligonucleotide such as Li28 is particularly appropriate for the treatment of chronic hepatitis B. The infecting HBV can be from the same genotype, strain or isolate as any HBV from which originates the HBV antigens in use in the present invention (e.g. genotype D) or it can be from a different genotype (e.g. genotype B, C, A or E).

Treatment of inflammatory diseases such as Alzheimer, arthritis (e.g. rheumatoid arthritis), asthma, atherosclerosis, Crohn disease, irritable bowel syndrome, systemic lupus erythematous, nephritis, Parkinson disease and ulcerative colitis can also be envisaged in the context of the present invention.

In a further aspect, the present invention also encompasses an immunostimulatory combination of the invention or a first composition for use according to the invention for inducing or stimulating an immune response according to the modalities described herein.

### Methods of Treatment

In another aspect, the present invention also relates to a method of treatment comprising administering to the subject (a) a first composition comprising a therapeutic vaccine as described herein and (b) a second composition comprising one or more oligonucleotide(s) as described herein in an amount sufficient to treat or prevent a disease or a pathologic condition in a subject in need thereof according to the modalities described herein. Preferably, said a) and b) steps are conducted sequentially with a specific preference for a) being 6-48 h (e.g. 24 h) before b).

In one embodiment, the disease or pathologic condition to be treated is a proliferative disease. Accordingly, the present invention also concerns a method for the treatment of a proliferative disease such as a cancer and a method for inhibiting tumor growth comprising administering at least (a) and (b) to a subject in need thereof. In another embodiment, the disease or pathologic condition to be treated is an infectious disease. Accordingly, the present invention also concerns a method for the treatment of an infectious disease such as hepatitis B caused by HBV infection and a method for treating a chronic HBV infection comprising administering at least (a) and (b) to a subject in need thereof.

In the context of the invention, the methods and use according to the invention aim at slowing down, curing, ameliorating or controlling the occurrence or the progression of the targeted disease or pathologic condition or alleviating one or more symptoms related to or associated with said disease or condition. Typically, upon administration according to the modalities described herein, the immunostimulatory combination or methods of the invention provide a therapeutic benefit to the treated subject which can be evidenced by an observable improvement of the clinical status over the baseline status or over the expected status if not treated with the combination described herein. An improvement of the clinical status can be easily assessed by any relevant clinical measurement typically used by physicians or other skilled healthcare staff. In the context of the invention, the therapeutic benefit can be transient (for one or a couple of months after cessation of administration) or sustained (for several months or years). As the natural course of clinical status which may vary considerably from a subject to another, it is not required that the therapeutic benefit be observed in each subject treated but in a significant number of subjects (e.g. statistically significant differences between two groups can be determined by any statistical test known in the art, such as a Tukey parametric test, the Kruskal-Wallis test the U test according to Mann and Whitney, the Student's t-test, the Wilcoxon test, etc.).

In a particular embodiment, when the method is aimed at treating a proliferative disease, in particular cancer, such a method of treatment can be correlated with an increase of the survival rate, a reduction in the tumor number; a reduction of the tumor size, a reduction in the number or extent of metastases, an increase in the length of remission, a stabilization (i.e. not worsening) of the state of disease, a delay or slowing of disease progression or severity, a prolonged survival, a better response to the standard treatment, an improvement of quality of life, a reduced mortality, etc., in the group of patients treated with the immunostimulatory combination of the present invention with respect to those non treated or treated with only one entity of the combination.

When the method aims at treating an infectious disease, a therapeutic benefit can be evidenced by, for instance, a decrease of the amount of the infecting pathogenic organism quantified in blood, plasma, or sera of a treated subject, and/or a stabilized (not worsening) state of the infectious disease (e.g. stabilization of inflammatory status), and/or the reduction of the level of specific serum markers (e.g. decrease of alanine aminotransferase (ALT) and/or aspartate aminotransferase (AST) associated with liver poor condition usually observed in chronic hepatitis B), decrease in the level of any antigen associated with the occurrence of an infectious disease and/or the appearance or the modification of the level of antibodies to the pathogenic organism and/or the release of signals by immune cells (e.g. cytokines) and/or an improved response of the treated subject to conventional therapies (e.g. antibiotics, nucleoside analogs, etc.) and/or a survival extension as compared to expected survival if not receiving the combination treatment.

The appropriate measurements such as blood tests, analysis of biological fluids and biopsies as well as medical imaging techniques can be used to assess a clinical benefit. They can be performed before the administration (baseline) and at various time points during treatment and after cessation of the treatment. For general guidance, such measurements are evaluated routinely in medical laboratories and hospitals and a large number of kits are available commercially (e.g. immunoassays, quantitative PCR assays). For example, the levels of HBV seromarker can be evaluated routinely in medical laboratories and hospitals and a large number of kits is available commercially (e.g. immunoassays developed by Abbott Laboratories, Organon Technika). In a specific embodiment, the method of the present invention permits to decrease the serum HBsAg level in a chronically infected patient by at least 0.5 log₁₀ and preferably by at least 0.7 log₁₀ (e.g. at least one log) for a suitable period of time (e.g. at least 2 months) as compared to before combi treatment. The present invention also relates to a method for decreasing HBV viral load in the serum of a subject diagnosed as having an HBV infection comprising administering the combination of the invention. For general guidance, the HBV viral load can be determined using a quantitative PCR assay or any other methodology accepted in the art (e.g. Roche Ampli Prep/Cobas taqman assay v2.0, Abbott real-time hepatitis B virus performance assay). In a specific embodiment, the method of the present invention permits to decrease the serum HBV DNA level in a chronically infected patient by at least 0.5 log₁₀ and preferably by at least 0.7 log₁₀ (e.g. for at least 2 months) as compared to before combi treatment.

### Method for Inducing an Immune Response

In a further aspect, the present invention also encompasses a method of inducing or stimulating an immune response comprising a) administering to a subject a first composition comprising an immunologically effective amount of a therapeutic vaccine as described herein and (b) administering to the subject a second composition comprising an immunologically effective amount of one or more oligonucleotide(s) as described herein. Preferably, said a) and b) are conducted sequentially with a specific preference for a) being 6-48 h (e.g. 24 h) before b).

In one embodiment, the induced or stimulated immune response can be specific (i.e. directed to epitopes/antigens) and/or non-specific (innate), humoral and/or cellular. In the context of the invention, the immune response is preferably a T cell response CD4+ or CD8+-mediated or both, directed to polypeptide(s)/epitope(s), in particular associated with a tumor.

The ability of the immunostimulatory combination and methods described herein to induce or stimulate an immune response can be evaluated either in vitro (e.g. using biological samples collected from the subject) or in vivo using a variety of direct or indirect assays which are standard in the art. For a general description of techniques available to evaluate the onset and activation of an immune response, see for example Coligan et al. (1992 and 1994, Current Protocols in Immunology; ed J Wiley & Sons Inc, National Institute of Health or subsequent editions). Several assays can be used to detect immune responses including, e.g. ELISA (enzyme-linked immunosorbent assay), ELISpot (enzyme-linked immunospot) and ICS (intracellular cytokine staining), multiparameters flow cytometry. The ability to stimulate a humoral response may be determined by antibody binding and/or competition in binding (see for example Harlow, 1989, Antibodies, Cold Spring Harbor Press). One may also use various available antibodies so as to evaluate the representativity and/or the level of activation of different immune cell populations involved in immune response, such as cytotoxic T cells, natural killer cells, macrophages, dendritic cells, etc. using surface markers detection. Evaluation of cellular immunity can be performed for example by quantification of cytokine(s) produced by activated T cells including those derived from CD4+ and CD8+ T-cells. Cytokine profile analysis can also be performed, e.g. by multiplex technologies or ELISA; proliferative capacity of T cells can be determined by e.g. by [³H] thymidine incorporation assay; cytotoxic capacity for antigen-specific T lymphocytes can be assayed in a sensitized subject or by immunization of appropriate animal models.

In a particular embodiment, the immunostimulatory combination and method(s) of the invention may be employed according to the modalities described herein to induce or enhance the innate immune response. Said induction or enhancement of the innate immune response is preferably correlated with an increase of immune effector cells and/or a change in the cytokine environment, especially at or at close proximity of the injection site. Said induction or enhancement of the innate immune response is preferably correlated with at least one (preferably 2 or 3) of the following properties:
An increase in the number of macrophages at or at close proximity of the injection site (e.g. at least 1.5-fold increase, preferably at least 2-fold increase; more preferably at least 2.5-fold increase and even more preferably at least 2.8-fold increase at least 24 h after injection of the immunostimulatory combination);
An increase in the number of activated CD69+ NK (natural killer) cells at or at close proximity of the injection site (e.g. an increase in the percentage of activated CD69+ NK cells by a factor of at least 1.5, advantageously at least 2, desirably at least 3, preferably at least 4, more preferably at least 5, and even more preferably at least 6, at least 24 h after injection of the immunostimulatory combination);
An increase in the number of KLRG1 (killer cell lectin receptor) positive CD3+ CD8+ lymphocytes at or at close proximity of the injection site (e.g. an increase of at least 10% in the percentage of KLRG1+ CD3+ CD8+ lymphocytes, at least 24 h after injection of the immunostimulatory combination);
An increase in the number of activated DC (dendritic cells) in the lymph node draining the injection site (e.g. an increase of a factor of at least 1.5 in the number of activated DCs at least 24 h after injection of the immunostimulatory combination);
An increase of the concentration of IL-18 at or at close proximity of the injection site (e.g. an increase of at least a factor 1.5, advantageously at least 2, desirably at least 3, preferably at least 4, more preferably at least 5, and even more preferably at least 10 in the concentration of IL-18, at least 24 h after injection of the immunostimulatory combination); and/or
An increase of the concentration of IL-1β at or at close proximity of the injection site (e.g. an increase of at least a factor 1.5, preferably at least 2, in the concentration of IL-1β, at least 24h after injection of the immunostimulatory combination); or
Any combination of two or more such properties.

In any of the methods according to this aspect of the invention, the immunostimulatory combination of the present invention can be administered in association with any conventional therapeutic modalities which are available for treating or preventing the targeted disease or pathological condition. Such conventional therapy may be administered to the subject concomitantly, prior to or subsequent to the immunostimulatory combination or method according to the invention.

Representative examples of conventional therapy include, without limitation, chemotherapy conventionally used for treating cancers, antibiotics, antimetabolites, antimitotics, antivirals, cytokines, chemokines, monoclonal antibodies, cytotoxic agents as well as siRNA and antisense polynucleotides (to inhibit expression of cellular genes associated with the targeted disease). According to an advantageous embodiment, especially when the therapeutic vaccine is armed with a suicide gene, the immunostimulatory combination or methods of the present invention may be used in association with the corresponding prodrug (see Table 1). The prodrug is administered in accordance with standard practice (e.g. per os, systematically, etc.).

Alternatively or in combination, the immunostimulatory combination or method of the invention can also be used in association with radiotherapy. Those skilled in the art can readily formulate appropriate radiation therapy protocols and parameters (see for example Perez and Brady, 1992, Principles and Practice of Radiation Oncology, 2nd Ed. JB Lippincott Co; using appropriate adaptations and modifications as will be readily apparent to those skilled in the field). The types of radiation that may be used in cancer treatment are well known in the art and include electron beams, high-energy photons from a linear accelerator or from radioactive sources such as cobalt or cesium, protons, and neutrons.

According to an advantageous embodiment, especially when the therapeutic vaccine encodes HBV antigens, the combination and methods of the present invention may be used in association with a standard of care. Representative examples of such standard of care include without limitation cytokines (e.g. IFNalpha, pegylated IFNa2a or 2b such as Pegasys (Roche), Pegintron (Schering Plough) or IntronA (Schering Plough)) and nucleos(t)ide analogs (NUCs) such as lamivudine, entecavir, telbivudine, adefovir, adefovir dipivoxil or tenofovir. The treatment with NUCs is only partially effective (infection resolution is observed in only 3-5% of subjects after 1 year of treatment) and needs long term therapy (may be life-long). It is expected that association with the immunostimulatory combination of the invention brings an immune dimension that would permit to complement NUC's action on viral replication, thus resulting in an improvement of such treatment (e.g. by decreasing doses of NUCs or length of NUC treatment required to achieve a therapeutic benefit) or an increase of the percentage of infection resolution (e.g., greater than 5%).

In another aspect, the present invention also provides a kit of parts comprising a) the first composition and b) the second composition comprised in the immunostimulatory combination of the invention together with instructions for use. In one embodiment, a kit includes at least the first composition (therapeutic vaccine) as discussed herein in one container and the second composition (one or more oligonucleotide(s)) as described herein in another container. Such containers are preferably sterile glass or plastic vial. A preferred kit comprises a MVA-based therapeutic vaccine (e.g. a MVA virus expressing the tumor-associated MUC1 antigen and the human IL-2) and Litenimod oligonucleotide. Another preferred kit comprises an Ad-based therapeutic vaccine (e.g. an Ad5 virus expressing HBV antigens such as the one described in WO2013/007772) and Litenimod oligonucleotide. Optionally, the kit can include suitable devices for performing the administration of each of the active agents and/or a package insert including information concerning the individual components and dosage.

In a further aspect, the present invention provides a method for treating a chronic infectious disease, such as a chronic hepatitis B, comprising one or more administration of a composition comprising a therapeutically or an immunologically effective amount of an oligonucleotide having at least 21 nucleotides in length and comprising at least three hexameric motifs represented as RRCGYY (SEQ ID NO:13) or RYCGYY (SEQ ID NO:14), wherein each R occurrence is a purine nucleotide or a purine nucleotide derivative; C is a cytosine nucleotide or a cytosine nucleotide derivative; G is a guanosine nucleotide or a guanosine nucleotide derivative; and Y is a pyrimidine nucleotide or a pyrimidine nucleotide derivative. Therefore, the present invention also relates to such a oligonucleotide composition for use for treating or preventing an infectious disease, especially a chronic infection disease such as a chronic hepatitis B. In a preferred embodiment, said oligonucleotide comprises a nucleotide sequence as shown in SEQ ID NO: 10 or a nucleotide sequence as shown in SEQ ID NO: 11.

All of the above cited disclosures of patents, publications and database entries are specifically incorporated herein by reference in their entirety. Other features, objects, and advantages of the invention will be apparent from the description and drawings, and from the claims.

### EXAMPLES

TG4010, MVATG9931 with its research name, is a therapeutic cancer vaccine based on a modified vaccinia virus Ankara (MVA), coding for MUC1 tumor-associated antigen and human interleukin 2 (IL-2). TG4010, in combination with first-line standard of care chemotherapy in advanced metastatic non-small-cell lung cancer (NSCLC), demonstrated efficacy in two different randomized and controlled phase 2b clinical trials (Quoix et al., 2011, The Lancet Oncol 12(12): 1125-33).

In the present study, we combined MVATG9931 with the synthetic CpG type B TLR9 ligand called Litenimod (Li28 or CpG-28). This molecule was successful in the treatment of intracranial gliomas in rats (Carpentier et al., 2000, Clinical cancer Res; 6(6): 2469-2473) and was clinically tested by intracerebral administration in patients with recurrent glioblastoma (Carpentier et al., 2010, Neuro-oncology 12(4):401-408).

The combination of MVATG9931 and Li28 in the prophylactic RMA-MUC1 model markedly increased survival in the subcutaneous RMA-MUC1 tumor model compared to the treatment with MVATG9931 or Li28 alone. We analyzed local cytokine and chemokine profiles and leukocyte populations around the injection site to identify features correlating with the observed anti-tumor effects. Besides the antigen-specific response provided by MVATG9931, local factors seemed of great importance for the observed effect. We observed a strong increase of the percentage of macrophages, the secretion of IL-18 and IL-1 beta and an increase of the percentage of activated CD69⁺ NK cells around the injection site. In vivo depletion of macrophages around the injection site by Clodronate liposomes reduced local IL-18 levels and diminished survival rates significantly. CD8⁺ T cells, accumulating at the MVA injection site, showed higher percentage of KLRG1⁺ cells upon combination treatment with Li28. Thus, MVATG9931 and Li28 together create adaptive and innate responses around the injection site superior to single component. Moreover, the efficacy of MVATG9931 and Li28 combinations were also compared to MVATG9931 combination involving either a TLR3 ligand consisting of the double-stranded RNA from yeast viruses, stabilized by the cationic lipid Lipofectin (NAB2+Lipofectin) (Claudepierre et al., 2014, J. Virol. 88(10): 5242-55), or the murine CpG B-type TLR9 ligand ODN1826 (Fend et al., 2014, Cancer Immunol. Res. 2(12): 1163-74) for which better survival and tumor rejection were observed in the RMA-MUC1 tumor model. In these two combination treatments, the major role of MVA was to promote the infiltration of CD8⁺ T cells in virus infected tissues including the tumor (Preville et al., 2015, Oncoimmunol. 4(5): e1003013). The role of TLR3 or RIG-I ligands was the modulation of the tumor environment into an immune-supportive tissue as reviewed in Van der Boorn and Hartmann (2013, Immunity 39(1): 27-37) and Gajewski et al. (2013, Nature Immunology 14(10): 1014-22).

### Materials and Methods

### Reagents

MVATG9931 and the non-recombinant (empty) MVA MVATG33.1 are described in Claudepierre et al. (2014, J. Virol. 88(10): 5242-55). Litenimod (Li28) is a synthetic B-type CpG oligonucleotide with a phosphorothioate backbone and three CpG motifs (TAAACGTTATAACGTTATGACGTCAT; SEQ ID NO: 10). This TLR9 ligand was selected for its optimal efficacy both in mice and humans (Carpentier et al., 2010, Neuro-oncology 12(4): 401-8). Li28 was chemically synthesized and provided in clinical purity at a concentration of 10 mg/ml in saline solution (0.9% NaCl) by Oligovax Inc. (Paris, France).

### Mice and RMA-MUC1 Tumor Model

Murine RMA-MUC1 tumor cells are derived from C57BL/6 lymphoma cells RMA (Karre et al., 1986, Nature 319: 675-78) transfected with an expression plasmid for the human MUC1 gene (Graham et al., 1996, Intern. J. Cancer 65(5): 664-70). C57BL/6 mice were obtained from Charles River (L'Arbresle, Les Oncins, France). Animals were used between 6 and 10 weeks' age. Mice were vaccinated by up to three weekly subcutaneous injections of MVATG9931 and of Li28 (10 μg). One week after the last injection, mice received 5×10⁵ RMA-MUC1 tumour cells by subcutaneous injection. During the following 60 to 80 days, tumour rejection and animal survival was monitored.

Tumor growth was monitored with a caliper twice per week and estimated according to the formula: 4/3×π (length/2×width/2×thickness/2) and expressed in mm³. Tumor rejection and mouse 20 survival were recorded. Mice were sacrificed for ethical reasons when the tumor volume was superior to 2000 mm³. This study was conducted in compliance with EU directive 2010/63/EU for animal experiments.

### Cell Infiltration Studies and Detection of Local Cytokines and Chemokines

The flanks of C57BL/6 mice were shaved and subcutaneously injected with test compounds. Mice were sacrificed and 1 cm² of skin was excised around the injection site. For infiltration studies, up to 4 skin samples were cut into small pieces, transferred into PBS-containing C-type tubes (Miltenyi Biotec), mechanically dissociated (GentleMACS; Miltenyi Biotec) and filtered (70 μm). Axillary and inguinal lymph nodes draining the injection sites were isolated and crushed passing them through 70 μm filters. Cell suspensions were washed twice in PBS, living cells were identified using LIVE/DEAD Near IR or Aqua (Invitrogen) staining. Fc receptors were blocked with mouse anti-CD16/CD32 (clone 93), and cells were stained for 15 minutes at 4° C. with mouse antibodies against F4/80 (BM8), 7/4 (ab53453), Langerin (929F3.01), CD11c (N418 or HL3), mPDCA-1 (JF05-1C2.4.1), CD4 (clone RM4-5), CD86 (clone GL1), CD3e (145-2C11), CD8a (53-6.7), CD19 (ID3), CD45 (30-F11), CD45R (RA3-6B2), Ly6C (AL-21), Ly6G (1A8), NKp46 (29A1.4), CD103 (M290), CD69 (H1.2F3), CD11b (M1/70), and KLRG1 (2F1) provided by Abcam, BD Biosciences, BioLegend, Miltenyi Biotec, or Dendritics. Cells were analyzed on FACS® Canto A, FACS® Aria III (Becton Dickinson), Navios cytometer (Beckman Coulter) or MacsQuant (Miltenyi). Analyses were performed with DIVA (Becton Dickinson) or Kaluza® (Beckman Coulter) softwares.

For local cytokine and chemokine detection, two skin samples per mouse were cut into small pieces in 500 μl PBS in C-type tubes (Miltenyi Biotec), and mechanically dissociated (GentleMACS; Miltenyi Biotec). After centrifugation at 300 g, turbid supernatants were transferred in Eppendorf tubes and centrifuged at 18000 g in the cold, cleared supernatant was analyzed with Procartaplex mouse chemokine and cytokine multiplex kits using a MagPix device according to the manufacturer's recommendations.

### Depletion of Macrophages Using Liposomal Clodronate

Local macrophages were depleted using Clodronate Liposomes optimized for immediate phagocytosis (Buiting and Von Rooijen, 1994, Journal of Drug Targeting 2(5): 357-62). Five mg/ml Clodronate containing liposomes (Clodrosome, Encapsula NanoSciences LLC) were subcutaneously injected at the vaccination site, PBS liposomes (Encapsome, Encapsula NanoSciences LLC) with the same lipid composition (18.8 mg/ml L-α-Phosphatidylcholine and 4,2 mg/ml Cholesterol) served as control. The recommended volume for sc injection to deplete skin macrophage was 100 μl (Stratis et al., 2006, J. Clin. Invest. 116(8): 2094-2104).

### Immunohistochemistry

Skin samples containing the injection sites were cut out and fixed in 4% formaldehyde, dehydrated and embedded in paraffin. Five μm thick sections were rehydrated and stained with Hematoxylin and Eosin. Additional sections were stained with Rat IgG2a F4/80 antibody (CalTag, MF48000) or Rat IgG2a isotype control (BD Pharmingen, 559073), goat to rabbit-HRP and revealed with TSA-Cy3. Stained sections were scanned using NanoZoomer slide scanner and Calopix software.

### Statistical Analysis

Mouse survival was analyzed in a Log-rank test using Statistica software (StatSoft). Hazard ratio calculations were carried out to identify significant differences between groups. Mann-Whitney tests were performed for individual comparisons of two independent groups, and Kruskall-Wallis when comparing more than two groups. Wilcoxon's tests were performed for individual comparisons of paired groups. Statistical analysis was performed with Graph pad Prism 5. P-Values<0.05 were considered significant.

### EXEMPLE 1: Combination of MVATG9931 and the CpG Type B TLR9 Ligand Li28 in the prophylactic RMA-MUC1 Tumor Model

### A. Sequential Administration

In previous work, we have shown that three subcutaneous (s.c.) injections with MVATG9931 day 1, 7 and 14, followed by s.c. implantation of RMA-MUC cells one week later, led to a reduction of tumor growth and an increase in survival. Highest survival rates of around 60% were obtained with a viral dose of 5×10⁵ pfu (Claudepierre et al., 2014, J. Virol. 88(10): 5242-55). Subsequently, we could demonstrate that tumor control was dependent on in vivo MUC1 expression. UV-inactivated MVATG9931, unable to allow for MUC1 gene expression, had no effect on survival rates (data not shown). In Vivo Imaging System (IVIS) studies with a luciferase-encoding MVA demonstrated that gene expression at the injection site was transient: expression was highest between 6 and 12 hours after injection, and was undetectable after 2 days (data not shown). The depletion of CD8⁺ or CD4⁺ cells before tumor implantation abolished all positive effects on survival rates underlining the importance of these cell types for the observed vaccine effect (data not shown).

We combined the MVATG9931 vector with the TLR9 ligand Li28 to evaluate the impact of the combined treatment on the MVATG9931-induced antigen-specific response and the immune environment around the injection site. We used the prophylactic RMA-MUC1 tumor model injecting a sub-optimal dose of MVATG9931 (1×10³ pfu), 10 μg of Li28 were either co-injected or applied at the MVA injection site with a delay of 6 or 24 hours.

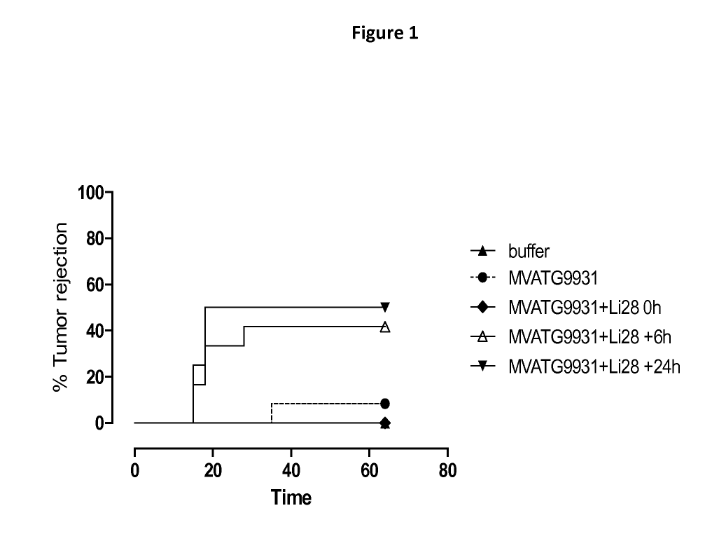

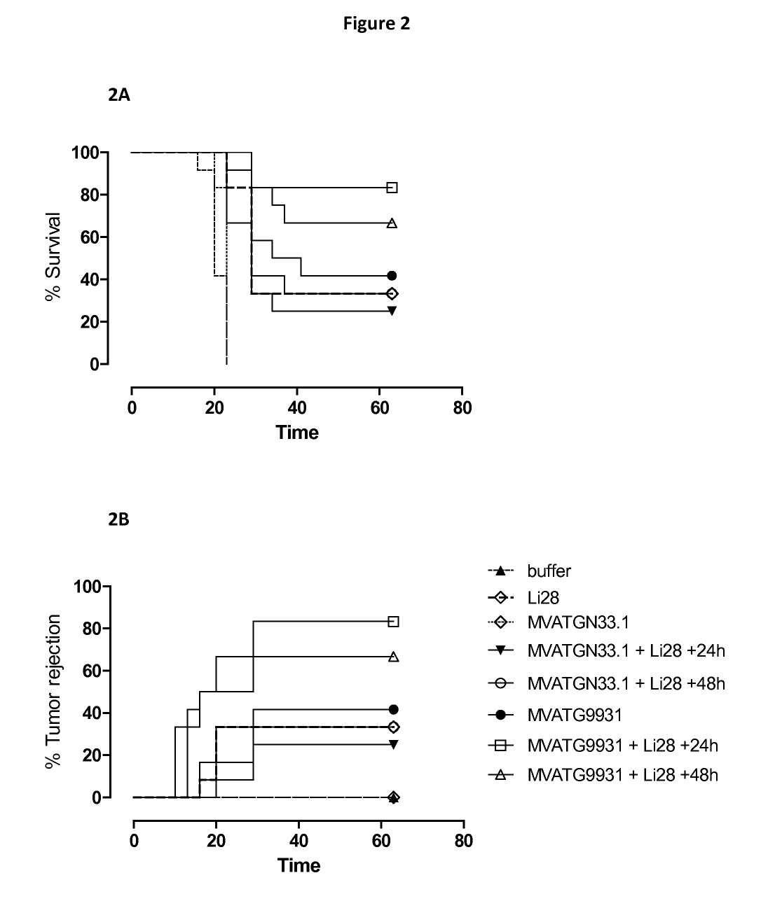

As illustrated in FIG. 1, co-injection did not improve tumor rejection rates whereas the sequential injections of MVA vector followed by Li28 showed beneficial effect. Indeed, the injection of Li28 either 6 h or 24 h after MVATG9931 significantly improved tumor rejection reaching levels of 50 and 40% respectively whereas no or very few tumor rejection was seen with the negative control (buffer), with MVATG9931 alone and with the co-injection of MVATG9931 and Li28. As illustrated in FIGS. 2, the beneficial effect of sequential injection was confirmed even when increasing the time interval between MVA and Li28 administrations. The increase in survival (FIG. 2A) and tumor regression (FIG. 2B) upon injection of Li28 24 hours after MVATG9931 was significant compared to MVATG9931, Li28, or the combination of Li28 with the empty MVA vector MVAN33.1. Injecting Li28 48 hours after MVATG9931 seemed also efficient but to a lesser degree. More specifically, survival of animals treated with the sequential combination reached about 85% (24 h time interval) and 70% (48 h time interval) whereas treatment with MVATG9931 alone gave 45% survival and 35% with the empty MVA control (N33). Sequential administration of MVATGN33.1 and Li28 (24 h and 48 h) did not provide any improvement as compared to MVATGN33.1 administration alone both in terms of survival and tumor regression and were less efficient than MVATG9931 alone (FIG. 2A and B). None of the mice treated with the buffer or Li28 survive.

### B. Administration Sites

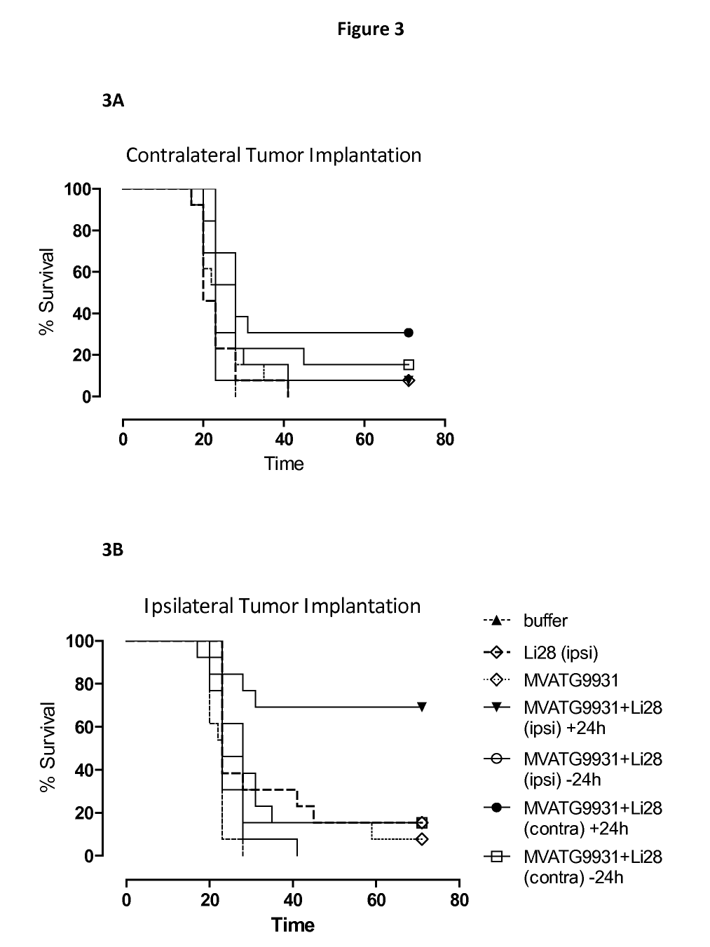

The importance of the administration site was also evaluated by comparing contralateral and ipsilateral tumor implantation (FIG. 3). When tumor cells were injected contralateral to the treatment with MVATG9931 and Li28, survival rates did not increase significantly (FIG. 3A). In marked contrast, highest effects of more than 80% survival rates were observed when MVATG9931, Li28 and the tumor cells were all injected in the same flank (ipsilateral) (FIG. 3B). Moreover, significant improvement of survival was obtained when Li28 was injected 24 h after MVATG9931 (+24 h) at the same site (ipsi). No effect was seen when injecting Li28 24 h before MVATG9931 (−24 h) whatever the site of injection (ipsi or contra) (FIG. 3B).

### C. Number of Injection cycles

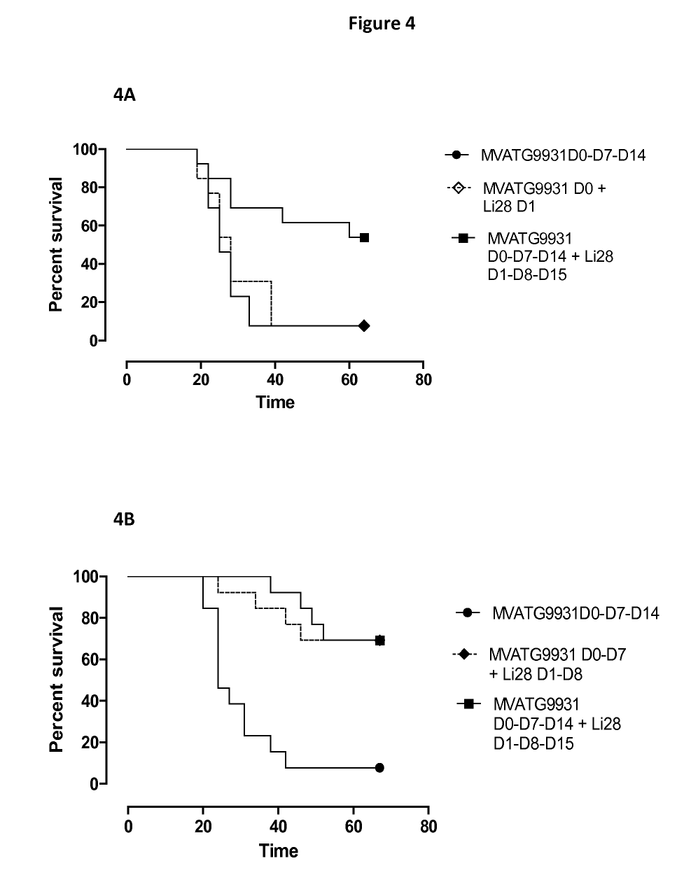

The injection schedule was evaluated further as illustrated in FIG. 4. Three injection cycles with MVATG9931 with (24 h after MVA injection) or without Li28 were compared to one or two injection cycles in the prophylactic RMA-MUC1 model. As shown in FIG. 4A, one injection cycle with both components (MVATG9931 D0+Li28 D1) resulted in survival rates observed with MVATG9931 alone, injected three times at low dose (MVATG9931 D0, D7 D14). Two injection cycles with MVATG9931 at 1×10³ pfu and Li28 (MVATG9931 D0-D7+Li28 D1-D8) were comparable to three injection cycles with both components (MVATG9931 D0-D7-D14+Li28 D1-D8-D15) (FIG. 4B).

The results indicate that the combination of MVATG9931 and Li28 strongly increased tumor control and survival rates. Besides the need for MUC1 expression, prerequisites for best effects were i) at least two vaccination cycles ii) injection of Li28 6-48 h (preference for 24 h) after MVATG9931 at the same site iii) and tumor implantation in vicinity (same flank) to the vaccination site.

### EXEMPLE 2: analysis of local cytokine, chemokine and leukocyte profile at injection site

### A. Local Characteristics of MVATG9931 Injection

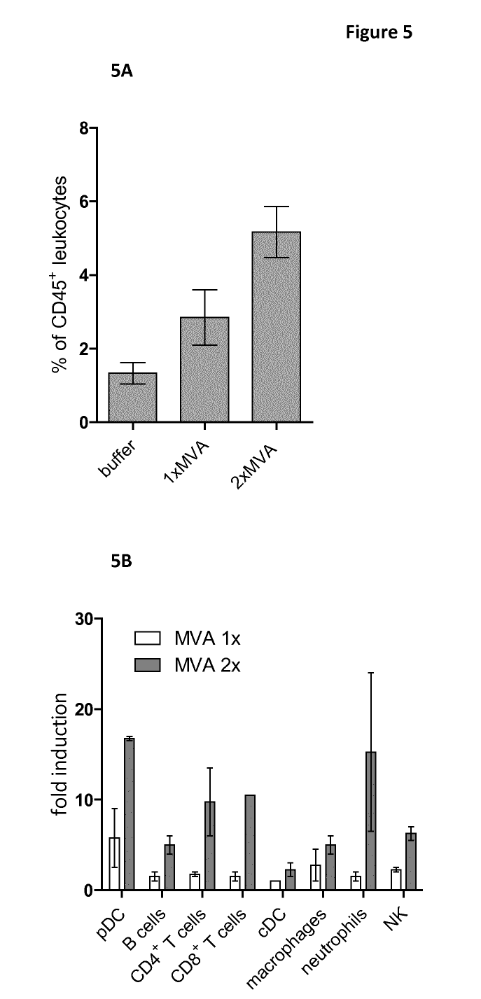

Various CD45⁺ cell populations were quantified at the injection site, 24 h after the first and second MVA injections. 5×10⁵ pfu of MVATG9931 were s.c. injected once or twice (D1 and D7). Twenty-four hours after the last injection (D2 or D8), mice were sacrificed, shaved skin samples comprising the injection sites were cut out and mechanically dissociated. Two skin samples per mouse from five to eight mice per group were pooled. Cell suspensions were stained for flow cytometry analysis: pDCs were identified as a Ly6C⁺mPDCA-1⁺CD45R⁺ CD11b⁻ subpopulation within living CD45⁺CD3⁻CD19⁻NKp46⁻ cells. Within the same sub-population, CD11c⁻CD11b⁺ cells were identified as Ly6G⁻ Ly6C⁺ F4/80⁺ macrophages or Ly6G⁺ Ly6C⁺ 7/4⁺ neutrophils. Within the CD45⁺CD3⁻CD19⁻NKp46⁻ population, CD11c⁺ cells were divided in cDCs (CD11b⁺) and dermal DCs (Langerin⁻). Within the CD45⁺CD11c⁻CD11b⁻ cell population, NK cells were identified as CD3⁻ and NKp46⁺, and B lymphocytes as CD3⁻ and CD19⁺ cells; CD8⁺ and CD4⁺ T lymphocytes were identified within the CD19⁻CD3′ cell population. The percentage of these various cell types within the total cell population was calculated, and the results were expressed as the fold induction on the basis of the values obtained with the buffer-injected control group. As shown in FIG. 5A, the percentage of CD45⁺ leukocytes in the skin after two injection cycles increased significantly by a factor of 3.8-fold (N=18). FIG. 5B illustrates the fold induction of percentages of various cell populations after one or two injections of MVA compared to buffer-injected control groups (n=2). Compared to the buffer-injected control, one single MVA injection increased the proportions of macrophages and NK cells 2 to 3-fold, and of pDCs 5-fold. Similarly, in comparison to buffer-injected control, 24h after the second MVA injection, proportions of CD4⁺ and CD8⁺ T lymphocytes, macrophages and NK cells increased 5 to 10-fold, while that of neutrophils and pDCs increased around 15-fold. In summary, after one single injection, pDCs, macrophages and NK cells augment. After the second MVA injection, all tested cell types except for cDCs and macrophages increase further compared to the first injection. The increase of percentages of the indicated cell population in the skin after two MVA injections compared to buffer control are significant (n=18) except for conventional DCs.

A time course for the percentages of CD45⁺ cells present around the injection site was established. Skin samples were taken day 8 after one single injection and 4 h, 24 h and 48 h after the second injection of MVATG9931 (5×10⁵ pfu). Eight days after the first injection, all cell populations were at baseline compared to the buffer control. After the second injection, the percentage of NK and CD8⁺ T cells increased over time up to 48 hours after injection. In contrast, a clear peak of infiltration was observed after 24 hours for neutrophils, pDCs, macrophages, B cells and CD4⁺ T cells (data not shown). The infiltration profile was not dependent on MUC-1 since similar leukocyte profiles were observed after s.c. injection of MVAs encoding GFP or luciferase (data not shown). In conclusion, we have observed pronounced leukocyte infiltration after the second injection of a MVA vector, with proportions of neutrophils, macrophages, B cells and pDC culminating 24 h after the second MVA injection.

### B. Local Characteristics of Combination Treatment with MVATG9931 and Li28 In Vivo

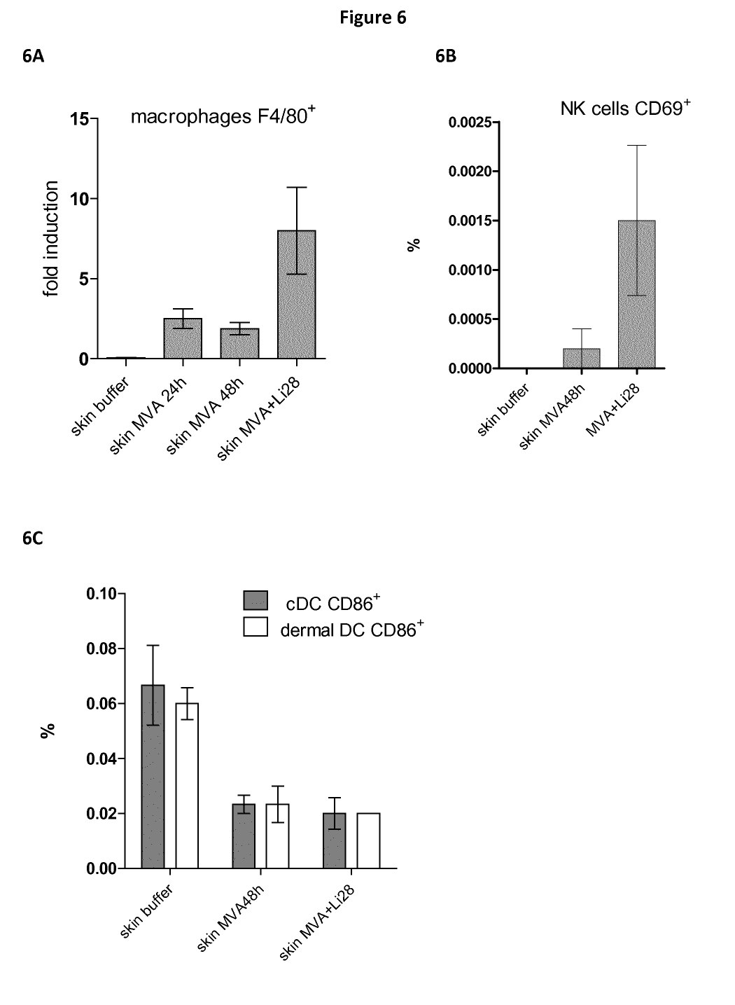

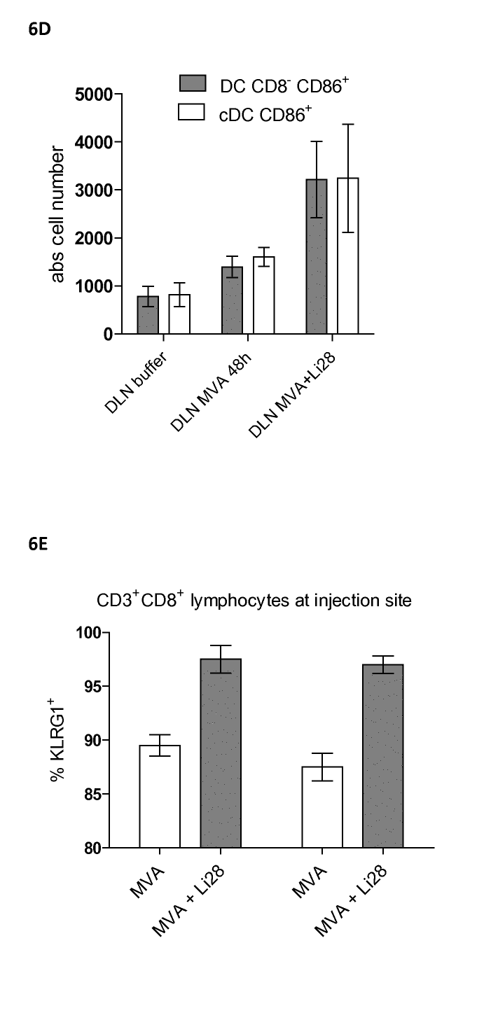

We investigated the effects of Li28 on MVA-induced cell infiltration profiles and the cell activation status at the injection site. After two treatment cycles with MVATG9931 (5×10⁵ pfu) and Li28 (10 μg), the cell populations around the injection site (skin) and in the draining lymph nodes (DLN) were isolated and characterized. Mice received two MVATG9931 s.c. injections (D1 and D7) and 24 h later two s.c. injections of 10 μg of Li28 (D2 and D8) at the same site. Mice were sacrificed 24 h after the second injection cycle (D9). Skin and draining lymph nodes were taken, single cell suspensions were generated and characterized by flow cytometry. A significant increase (fold induction of percentages) of macrophages (FIG. 6A) and activated CD69⁺ NK cells (FIG. 6B) at or close to the injection site (skin) was observed after MVATG9931 and Li28 treatment compared to treatment with MVATG9931 alone harvested after 24 h or 48 hours. The fold induction of macrophage percentages is about 3 whereas the percentage of activated CD69⁺ NK cells increased by a factor of about 7 after combinatorial treatment.

Further, within the CD45³⁰ CD3⁻CD19⁻NKp46⁻ population, CD11c⁺ cells were divided in conventional CD11b⁺ DCs (cDCs), and dermal CD11b^{-/low} DCs (Guilliams et al., 2010, European Journal of Immunology 40(8): 2089-94). Treatment with MVA alone or MVA+Li28 led to a decrease of activated CD86⁺ cDCs and CD86⁺ dermal DCs around the injection site (FIG. 6C) whereas, in the draining lymph nodes, the absolute number of CD86⁺ cDCs and a population of CD86⁺ CD8⁻ DCs increased in the MVA+Li28 treated group compared to the group treated only with MVA (FIG. 6D). In addition, lymphocytes extracted from the vaccination site were tested for CD8, CD3, KLRG1 and

CD127 expression. Compared to MVA treated control groups, Li28 treatment increased the percentage of KLRG1⁺ CD3⁺CD8⁺ lymphocytes at the MVA injection site (FIG. 6E).

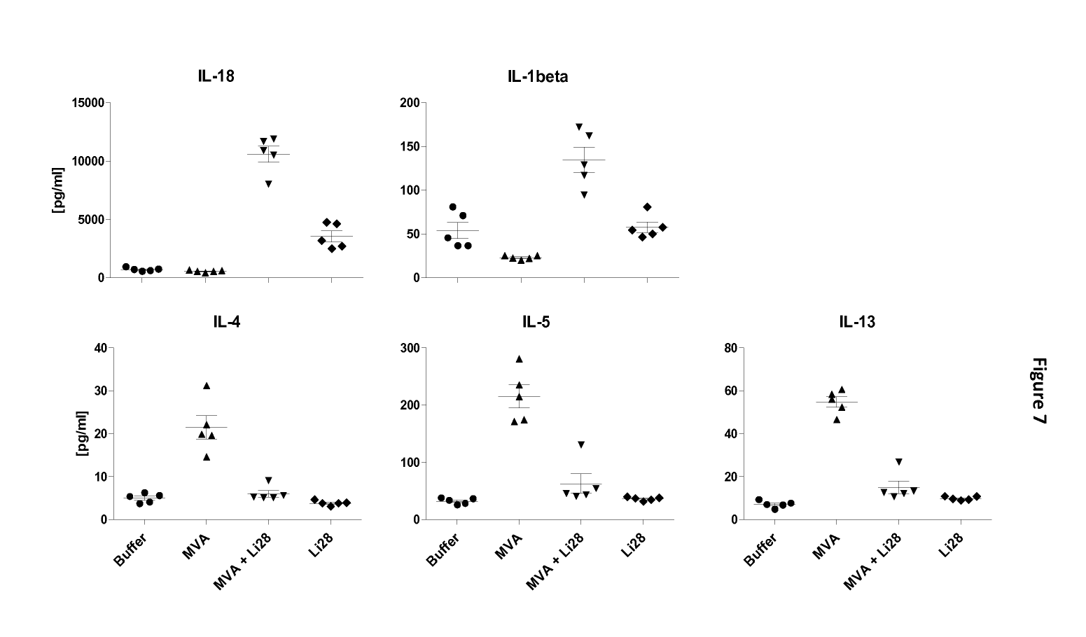

The analysis of local cytokine profiles showed that IL-18 and IL1-beta, not detectable or present at low levels after treatment with MVA or Li28 alone, increased significantly after two injection cycles with MVATG9931 and Li28 (FIGS. 7A and 7B). In contrast, treatment with MVA alone led to the secretion of IL-4, IL-5 and IL-13, undetectable after treatment with Li28 alone or after combination treatment (FIGS. 7C, D and E).

In conclusion, combinatorial treatment of MVATG9931 and Li28 increased the amount of macrophages and activated CD69⁺ NK cells at the injection site, increased the number of activated DCs in the draining lymph nodes, and increased the percentage of KLRG1-positive CD8⁺ cells accumulating at the MVA injection site. The cytokine profile after combination treatment changed and was characterized by IL-18 and IL-1b secretion around the injection site.

### C. Clodronate Treatment Eliminates Local Macrophages, Reduces Local IL-18 and Abolishes Anti-Tumor Effect

The effect of depletion of macrophages around the injection site was studied. To do this, Clodronate liposomes or the control PBS liposomes were injected 8 hours after the injection of 5×10⁵ pfu MVA9931+Li28 (+24h) at the same site, this injection cycle was repeated after one week. The day after the last injection, mice were sacrificed and the injection sites prepared for immunohistochemical studies. The isolated injection sites were fixed in 4% formaldehyde, dehydrated and embedded in paraffin, cut in 5 μm-thick sections. Skin structure was analysed with Hematoxylin and Eosin staining and macrophage staining with anti F4/80 (CalTag, MF48000). Immune-histochemical analyses showed that Clodronate liposomes had completely eliminated F4/80 macrophages, which had been readily detectable after combination treatment.

Looking at local cytokine profile around the injection site, we observed that Clodronate treatment reduced IL-18 levels and restored detectable levels of IL-4 and IL-5.

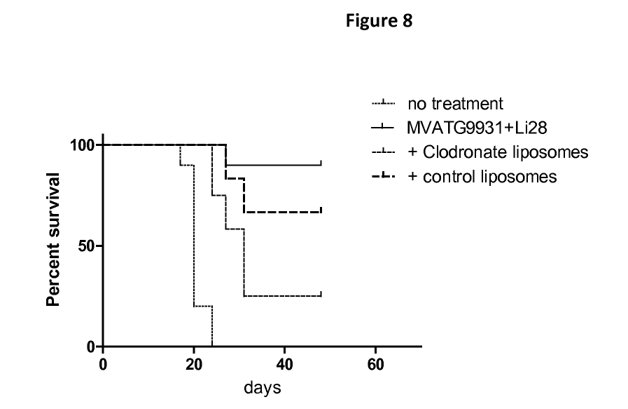

Depletion of macrophages by Clodronate liposomes around vaccination site was also evaluated in a tumor control experiment. Injection of 1×10³ pfu of MVATG9931 day 1 and 6, followed by 10 μg Li28 in the morning of day 2 and 7, followed by injection of 60 μl Clodronate liposomes or control liposomes in the evening of day 2 and 7. Survival rates obtained were followed in each group. As shown in FIG. 8, survival rates obtained with MVATG9931 and Li28 were significantly reduced after Clodronate treatment.

Altogether, these data confirm the importance of macrophages for the combinatorial treatment with MVATG9931 and Li28.

### EXEMPLE 3: Combination of Western Reserve Vaccinia Virus and Li28 in Murine Bone Marrow Derived Macrophages (m-CSF)

C57BL/6 mice were sacrificed, bone marrow cells were isolated and differentiated to murine bone marrow derived macrophages during 8 days in the presence of m-CSF (100 μg/ml) in RPMI 10% fetal calf serum. 5×10⁵ murine macrophages were plated in 500 μl RPMI in 24 well plates and infected with either a MVA vector expressing GFP (MVA-GFP) or with a TK- and RR-oncolytic Vaccinia virus of Western Reserve strain (WR-GFP) at MOI of 0.1, 0.3 or 1. Two hours later Li28 was added and the percentage of GFP positive cells were determined (N=2) by flow cytometry. As an alternative, immune-modulator, NAB2+Lipofectin (Claudepierre et al., 2014, J. Virol. 88(10): 5242-55) was also tested.

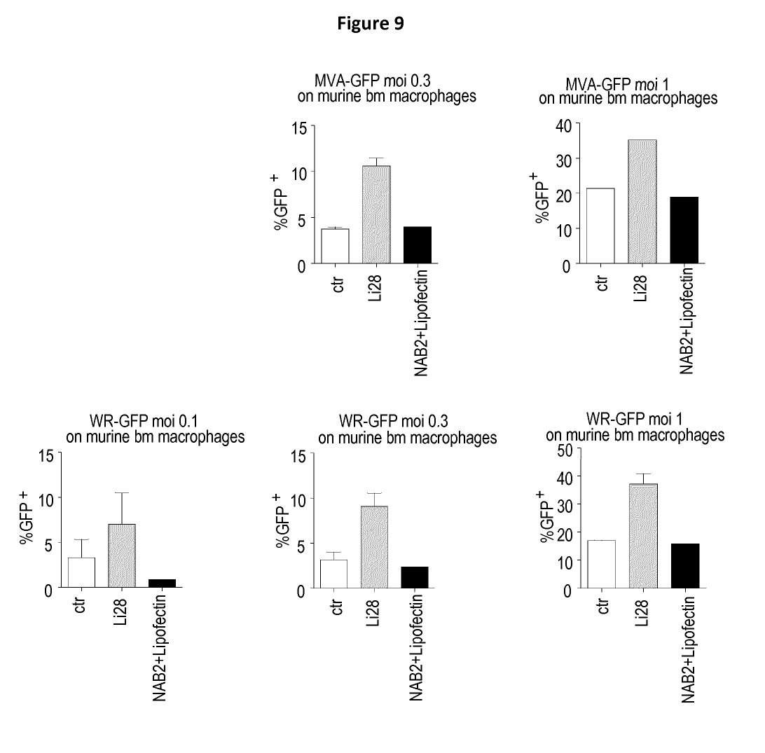

As shown in FIG. 9, the combination of Li28 increases infection rates of macrophages with MVA-GFP as well as with WR-GFP at MOI 0.3. A similar increase in the percentage of GFP positive murine macrophages was also seen at MOI of 0.1 and 1.

### EXEMPLE 4: Comparison with Other CpG Oligonucleotides and TLR Ligand

As shown in FIG. 9, in vitro infection of murine bone-marrow derived macrophages with the combinatorial approach involving MVA-GFP or WR-GFP with Li28 showed higher infection rates than with the TLR3 ligand NAB2 described in Claudepierre et al. (2014, J. Virol. 88(10): 5242-55).

The anti-tumor protection provided by various CpG TLR9 agonist oligonucleotides (available from Invivogen) in combination with MVATG9931 was evaluated in the prophylactic RMA-MUC1 tumor model. More specifically, ODN1585 and ODN2336 are Class A-type TLR9 ligands, ODN1826 and ODN2006 are Class B-type TLR9 ligands whereas ODN2395 is Class C.

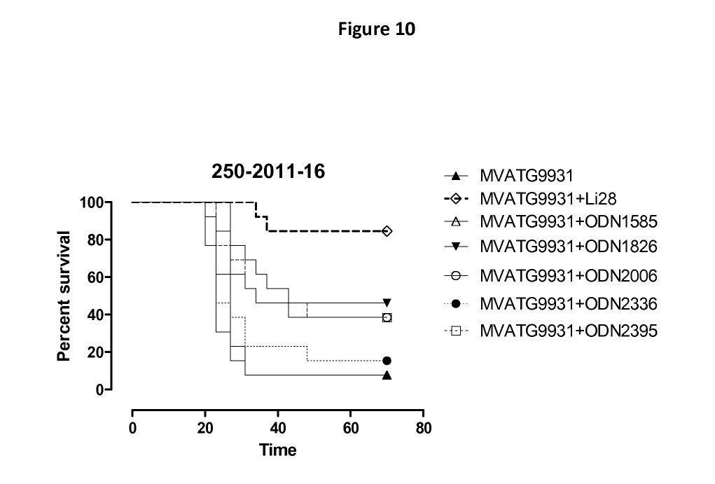

As illustrated in FIG. 10, combinatorial approaches with Li28 showed higher tumor protection than the other TLR9 ligands with about 85% survival as compared to 15% protection in combination with ODN2336, between 40-50% with ODN2395, ODN2006 and ODN1826 and 8% with MVATG9931 alone or in combination with ODN1585.

### Discussion

We have shown that the combination of an MVATG9931 with Li28 changes the environment around the injection site to an antigen-specific tumor-hostile environment. Combination treatment was defined by the higher frequency of macrophages at the injection site. Macrophage infiltration is a hallmark of chronic treatment with CpG-oligonucleotides (Mathes et al., 2015, Experimental Dermatology 24(2): 133-9). We have achieved strong macrophage infiltration with comparatively low doses of Li28 when associated with MVA. Local MVA infection induces chemo-attraction of macrophages, B cell, pDCs and neutrophils. IL-18 observed after combination treatment in vivo seems to stem mainly from macrophages since their depletion reduced the local level of this cytokine. Even though we had observed macrophages after injection of Li28 alone, we did not detect IL-18. This suggest that the macrophage phenotype might be altered by the combination treatment. We suggest that IL-18 activates NK cells, for example with the intermediate of DCs (Brandstadter et al., 2014, Eur. J. Immunol. 44(9) 2659-66). Further, TLR9 stimulation of pDCs contributes to macrophage attraction and stimulation of NK cells (Guillerey et al., 2012, Blood 120(1): 90-9). Activated NK cells are supposed to play a major role in the control of the nearby implanted tumor cells (for review Pahl and Cerwenka, 2015, Immunobiology).

Antigen-specific tumor control by MVATG9931 in the prophylactic RMA-MUC1 model clearly depends on transient de novo expression of MUC1 and CD8⁺ and CD4⁺ T cells. The combination treatment of MVATG9931 with Li28 was still MUC-1-dependent, however, this response was not systemic since contralateral implanted tumors were not controlled. Dendritic cells as well as 5 macrophages are described to function as antigen presenting cells after MVA infection (Abadie et al., 2009, PLoS One 4(12): e8159). The MVA-induced transient transgene expression coincides with the Li28 treatment, both treatments together might improve the antigen presentation by macrophages. Further, combination treatment increased the number of activated CD86⁺ dendritic cells in the draining lymph nodes. However, we were not able to demonstrate an increase of MUC1⁺ specific responses in an IFN-γ Elispot in splenocytes (data not shown). Nevertheless, the constant level of local RANTES after second MVA infection suggests support for CD8 T cell responses during this “chronic” viral infection (Crawford et al., 2011, PLoS pathogens 7(7): e1002098). So far we could demonstrate a Li28-induced increase of the percentage of KLRG1⁺ CD8 T lymphocytes around the MVA injection site. These cells were CD127-negative, suggesting that Li28 increased locally effector activity of T cells. Intracellular cytokine secretion assays studies are necessary to monitor antigen-specificity of these cells.

In the prophylactic RMA-MUC1 model, we are bringing the tumor cells close to the vaccination site representing an immunotherapeutic microenvironment. In the real life, we will have to induce this type of immunotherapeutic environment at the tumor site. Intratumoral injection of CpG-oligonucleotides in 9 L gliomas induced initial tumor growth inhibition due to an implication of macrophages (Auf et al., 2001, Clinical cancer Res 7(11): 3540-3). To this, high doses of CPG-oligonucleotides (50-100 μg) had to be injected repeatedly intratumorally to observe an effect bearing the risk of severe side effects like macrophage activation syndrome (MAS) (Behrens et al., 2011, J. Clin. Invest. 121(6): 2264-77). We propose that the intratumoral application of MVA or a vaccinia viral vector and Li28 could be beneficial in two ways: Firstly, the virus-induced infiltration of CD8⁺ T cells in the tumoral injection site accompanied by a tumor antigen-specific response to tumor encoded antigens liberated after virus-induced cell-death. Secondly, injection of low amounts of CpGs avoiding the risk of severe side effects due to leakage.

We have dissected local characteristics of the combination treatment using a MVA tumor vaccine and Li28 and showed that together they create a new type of immune microenvironment.

### EXEMPLE 5: Combination of AdTG18201 and the CpG Type B TLR9 Ligand Li28 in an HBV Persistent Mouse Model

### 5.1 Materials and Methods

The construction described below is carried out according to the general genetic engineered and molecular cloning techniques detailed in Maniatis et al. (1989, Laboratory Manual, Cold Spring Harbor Laboratory Press, Cold Spring Harbor N.Y. or subsequent editions) or according to the manufacturer's recommendations when a commercial kit is used. PCR amplification techniques are known to the person skilled in the art (see for example PCR protocols—A guide to methods and applications, 1990, published by Innis, Gelfand, Sninsky and White, Academic Press).

### Vector Construction and Production

TG1050 (or AdTG18201 under its research name) illustrated hereinafter was engineered to express a fusion of a truncated Core polypeptide (aa 1-148) (called Coret) with a mutated polymerase polypeptide (designated Pol*) comprising two internal deletions (from positions 538 to 544 and from positions 710 to 742) and 4 amino acid substitutions (D689H, V769Y, V776Y and D777H respectively) and with two immunogenic Env domains (Env1 and Env2, respectively extending from amino acids 14 to 51 and from amino acids 165 to 194 of the HBs protein) inserted in place of the deleted pol regions (as represented in SEQ ID NO:8 of WO2013/007772). All originate from HBV strain Y07587 which sequence is described in international databases (Gen bank Y07587) and in different publications. It is a genotype D virus of serotype ayw.

More specifically, a synthetic gene encoding a Coret-Pol-Env1-Pol-Env2-Pol fusion protein was synthesized by GEN EART (Regensburg, Germany). This fragment was inserted into the Nhel and Notl restriction sites of an adenoviral shuttle plasmid (pTG13135) containing a CMV-driven expression cassette surrounded by adenoviral sequences (adenoviral nucleotides 1-454 and nucleotides 3513-5781 respectively) to allow further generation of the vector genome by homologous recombination (Chartier et al., 1996, J. Virol. 70:4805). The resulting plasmid was called pTG18188.

An adenoviral vector was then obtained by homologous recombination between pTG18188 digested by Bst1107l and Pacl and pTG15378 (encoding the complete adenoviral genome) linearized by Clal digestion. This final adenoviral vector is E3 (nucleotides 28593-30464) and E1 (nucleotides 455-3512) deleted, with the E1 region replaced by the expression cassette containing, from 5′ to 3′, the CMV immediate-early enhancer/promoter, a chimeric human β-globin/IgG intron (as found in pCl vector available in Promega), the synthetic gene sequence encoding the Coret-Pol-Envl-Pol-Env2-Pol and the SV40 late polyadenylation signal. The resulting adenoviral vector (AdTG18201) was generated by transfecting the Pacl linearized viral genomes into an E1 complementation cell line. Virus propagation, purification and titration was made as described in Erbs et al. (2000, Cancer Res. 60: 3813). AdTG18201 is described in Martin et al. (Gut, 2015, 64(12): 1961-71) and in WO2013/007772).

### Antiviral and Immunological Responses Evaluation in a Mouse Model

### HBV-Persistent Mouse Model

The HBV persistent mice used in the study were described by Dion et al. (2013, J Virol, 87(10):5554-63). The model is based on the introduction in mice of an adeno-associated virus (AAV) encoding for a full-length HBV genome (AAV2/8-HBV) and causing the production of infectious HBV particles in mouse livers. This allows the analysis of HBV-specific viral parameters (HBsAg, HBeAg, HBcAg and viremia) as well as immunological read-outs (ICS, ELISpot or humoral immune responses).

More specifically, in the study described here, C57BL/6J mice were infected with 5×10¹⁰ vg of AAV2/8-HBV in the retro-orbital venous sinus. Blood samples were taken before treatment (at days 14 and 28 after AAV2/8-HBV infection, sera were sampled to allocate mice per group based on their level of HBsAg at those times before treatment start). Blood samples were also taken after treatments for about 3 months (at days 14, 28, 42, 56, 70 and 84 after the 1st TG1050 injection).

### Administration Protocols

Mice were subcutaneously (sc) immunized with 2×10⁹ vp of AdTG18201 (once weekly for 3 weeks, administration at days 0, 7 and 14).

CpG, ODN1826 (Invivogen) or Litenimod (Li28, provided by Oligovax) was administered intraperitoneally (once weekly for three weeks on days 9, 16 and 23, 100 μL (corresponding to 20 μg/injection)). Lyophilized ODN 1826 (200 μg) was diluted to 200 μg/mL with sterile PBS. Li28 was provided by Oligovax as a frozen solution at a concentration of 10 mg/mL of Li28 (in 0.9% sterile Na/Cl).

### Immunological Parameter Monitoring

### Peptides used for ELISpot Assay

Peptides used for cell stimulation ex vivo are short peptides of 9 to 10 amino acids. Peptides corresponding to described H-2^{b}-restricted epitopes of Pol protein VSA (position 419 to 428, VSAAFYHLPL; SEQ ID NO: 19) and DNA binding protein of Adenovirus FAL (FALSNAEDL; SEQ ID NO: 20) were synthesized by Proteogenix SAS (France) and were dissolved in 100% DMSO (Sigma) at a concentration of 10 mM.

### IFNg ELISpot Assay

Splenocytes from mice were collected at day 118 following AAV-HBV injection (corresponding to 84 days after the 1st AdTG18201 injection) and red blood cells were lysed (Sigma). 2×10⁶ cells per well were cultured in triplicate for 40 h in Multiscreen plates (Millipore, MSHA) coated with an anti-mouse IFNγ monoclonal antibody (BD Biosciences; 10 μg/mL) in MEM culture medium (Gibco) supplemented with 10% FCS (JRH, 12003-100M), 80 U/mL penicillin/80 μg/mL streptomycin (PAN), 2 mM L-glutamine (Gibco), 1× non-essential amino acids (Gibco), 10 mM Hepes (Gibco), 1 mM sodium pyruvate (Gibco) and 50 μM β-mercaptoethanol (Gibco) and in presence of 10 units/mL of recombinant murine IL2 (Peprotech), alone as negative control, or with:
10 μM of a selected H-2^{b} restricted peptide present in HBV Polymerase called VSA (SEQ ID NO: 19) or an adenovirus specific peptide (FAL; SEQ ID NO: 20)
5 μg/mL of Concanavalin A (Sigma) for positive control.

IFNg-producing T cells were quantified by cytokine-specific ELISpot (enzyme linked immunospot) assay as previously described (Himoudi et al., 2002, J. Virol. 76: 12735). The number of spots corresponds to the number of IFNg-producing cells. Results are shown as the mean value obtained for triplicate wells for each mouse and mean value per group. An experimental threshold of positivity for observed responses (or experimental cutoff) was determined by calculating a threshold value which corresponds to the mean value of spots observed with medium alone +2 standard deviations, reported to 10⁶ cells. A technical cutoff linked to the CTL ELISpot reader was also defined as being 50 spots/10⁶ cells (which is the value above which the CV (coefficient of variation) of the reader was systematically less than 20%). The highest value between the technical cutoff and the experimental threshold calculated for each experiment was taken into account to define the cutoff value of each experiment.

### Viral Parameter Monitoring

HBsAg levels in mouse serum was assessed using a commercial ELISA kit (Monolisa HBsAg Ultra, Bio-Rad, France) according to the manufacturer's protocol, except that a standard curve has been established, which renders the test quantitative. Serum has been diluted 1/400, 1/2000, 1/10000 and 1/50000. The HBsAg concentration was calculated in ng/mL referring to a standard curve established with 8 known concentrations of rHBsAg (Hytest, subtype adr) giving a range of HBsAg concentrations between 0.2195 ng/mL and 3.75 ng/mL in PBS 1× 0.05% Tween 20.

### 5.2. Results

In this experiment, HBV carrier mice (having received one injection of AAV2/8-HBV) were divided in 6 groups of 8 animals which were treated differently. Group 1 was left untreated. Groups 2, 4 and 6 were immunized with 3 weekly subcutaneous injections of AdTG18201 (at days 0, 7 and 14). Groups 3 and 4 were treated by ODN1826 injected 3 times at day 9, 16 and 23 via intraperitoneal route. Groups 5 and 6 were treated by Li28 injected 3 times at day 9, 16 and 23 via intraperitoneal route. Thus, groups 4 and 6 received combination treatments, associating AdTG18201 with either ODN1826 or Li28.

Ability of individual treatments or combinations of treatments to impact the HBsAg level detected in sera was assessed all along the protocol as well as their ability to induce adenovirus and HBV-specific T cell immune responses detectable at the end of the protocol using mouse splenocytes.

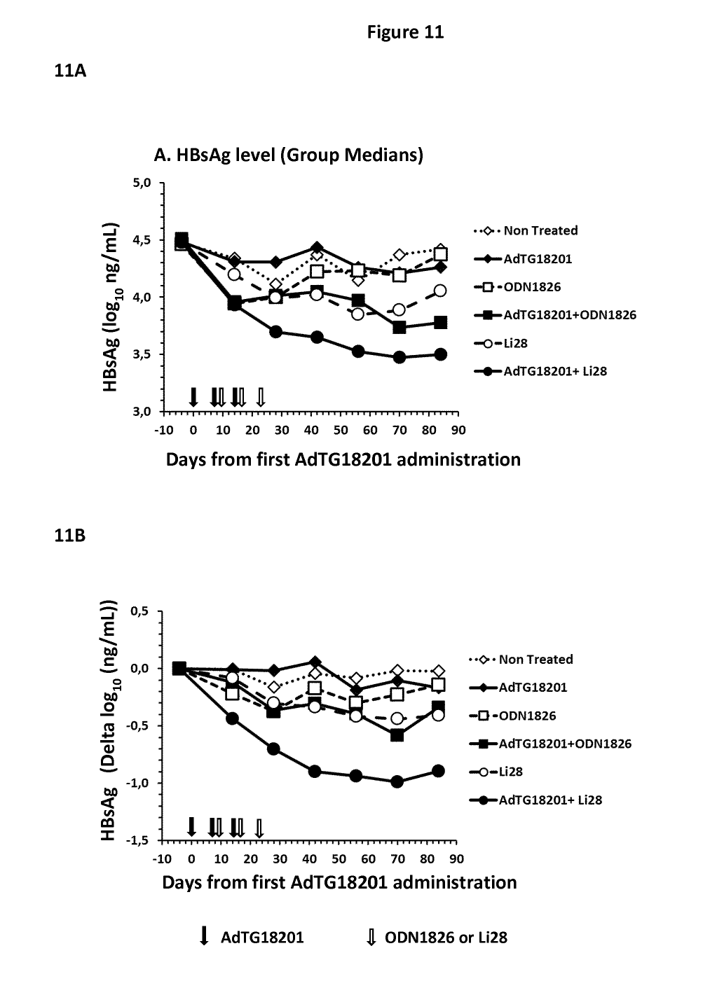

FIG. 11 shows the evolution of HBsAg levels in the sera of mice along time, FIG. 11A showing median values per group in ng/mL (log10) and FIG. 11B showing the median value per group of delta log for each time point compared to baseline. In these experimental conditions, AdTG18201 did not display any impact on HBsAg levels compared to untreated mice. Of note, a slight decrease in HBsAg level can be observed for some of the AdTG18201-treated mice in group 2 (not shown), which is however not reflected by the median value. A slight, very early and transient decrease was observed in mice treated by ODN1826 alone (max decrease of about 0.4 log (median)). For Li28 treated mice (group 5), a similar decrease can be observed (maximum decrease of about 0.5log (median)) and this decrease appears to be more sustained over time. Combination of AdTG18201 with ODN1826 (group 4) induced an HBsAg decrease which is stronger than each individual treatment (maximum decrease of about 0.6 log (median)), showing an interest to combine CpG such as ODN1826 with AdTG18201. The mice treated by the combination AdTG18201 and Li28 displayed the strongest HBsAg decrease with a maximum median value of decrease of about 1 log. This decrease is stronger than the one observed for each individual therapy. Of note, the group treated by AdTG18201+Li28 is the only one displaying 3 out of 8 mice with HBsAg levels below the limit of quantification (LLOQ) of the HBsAg assay at different time points. These results on HBsAg levels clearly demonstrated the higher potential of a combination therapy associating AdTG18201 and Li28.

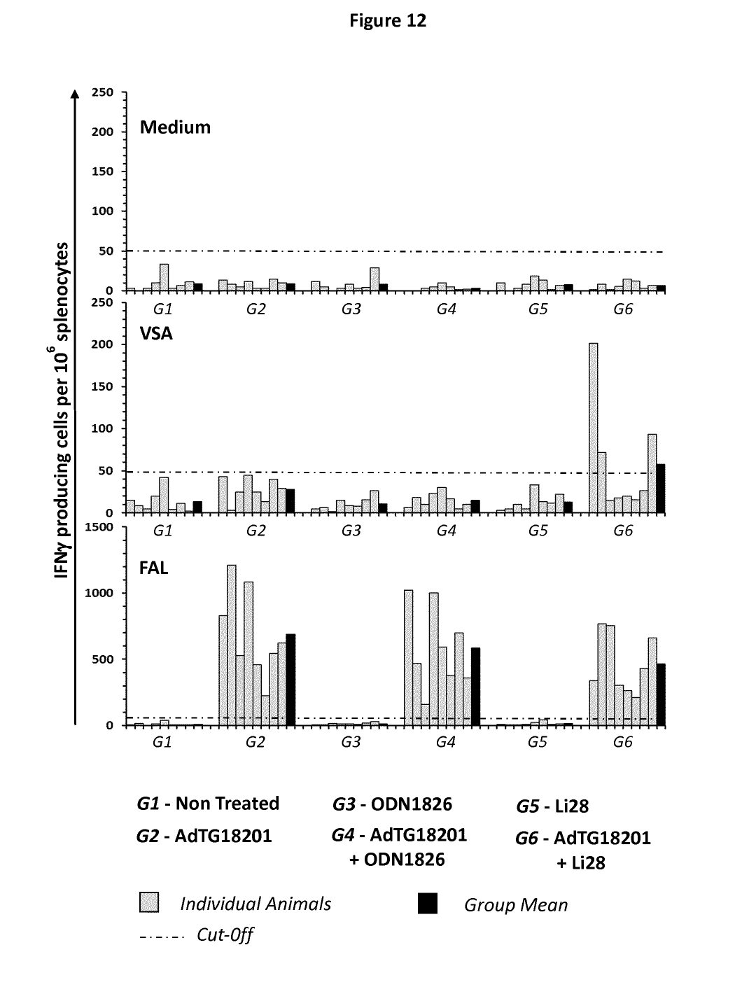

FIG. 12 shows the immune response monitored on spleen cells of mice by an IFNγ ELISPOT assay at the end of the protocol, 3 months after the start of the therapy(ies). IFNγ-producing cells were monitored in presence of medium (negative control), or of the FAL peptide (monitoring of Adenovirus-specific immune response) or of the VSA peptide (monitoring of HBV Polymerase specific immune response) or of Concanavalin A (ConA, positive control). All mice displayed high frequencies of IFNγ-producing cells following stimulation with ConA, this result validates the ability of cells of all mice to mediate an immune response and thus validate the experiment (not shown). No background was observed in the negative control condition with medium for all mice. All mice injected with the AdTG18201 displayed high and comparable frequencies of Adenovirus-specific IFNγ-producing cells (group 2, 4 and 6) whereas groups that did not receive any Adenovirus immunization did not display such responses (as expected). The group 6, treated by the combination of AdTG18201 and Li28 is the only one displaying detectable HBV-specific immune response with 3 mice with detectable frequencies of IFNγ-producing cells specific of the VSA peptide from the HBV polymerase. These 3 mice were the ones which had HBsAg level below the LLOQ at some time points. Detection of an HBV-specific immune response at a late time point on spleen cells only in mice treated with the combination AdTG18201+Li28 highlights the interest of such a combination.

To conclude, this experiment shows the interest of combining AdTG18201 with a TLR9 agonist such as CpG, especially with Li28 for an HBV therapy. The combination of AdTG18201 and Li28 leads to the strongest decrease in HBsAg levels, is the only one allowing to detect HBV-specific T cell responses at the end of the protocol. These data are strengthened by the correlation between the strongest HBsAg decrease (values below LLOQ) and the detection of HBV-specific immune response at the end of the protocol for 3 out of the 8 mice treated by the combination AdTG18201+Li28.

1. An immunostimulatory combination comprising at least (a) a first composition comprising a therapeutically or an immunologically effective amount of a therapeutic vaccine and (b) a second composition comprising a therapeutically or an immunologically effective amount of an oligonucleotide having at least 21 nucleotides in length and comprising at least three hexameric motifs represented as RRCGYY (SEQ ID NO:13) or RYCGYY (SEQ ID NO:14), wherein each R occurrence is a purine nucleotide or a purine nucleotide derivative; C is a cytosine nucleotide or a cytosine nucleotide derivative; G is a guanosine nucleotide or a guanosine nucleotide derivative;
and Y is a pyrimidine nucleotide or a pyrimidine nucleotide derivative.

2. A first composition comprising a therapeutically or an immunologically effective amount of a therapeutic vaccine for use in the treatment of a disease in combination with a second composition comprising a therapeutically or an immunologically effective amount of an oligonucleotide; wherein said oligonucleotide has at least 21 nucleotides in length and comprises at least three hexameric motifs represented as RRCGYY(SEQ ID NO:13) or RYCGYY (SEQ ID NO:14), wherein each R occurrence is a purine nucleotide or a purine nucleotide derivative; C is a cytosine nucleotide or a cytosine nucleotide derivative; G is a guanosine nucleotide or a guanosine nucleotide derivative; and Y is a pyrimidine nucleotide or a pyrimidine nucleotide derivative.

3. The immunostimulatory combination of claim 1 or the first composition for use according to claim 2, wherein said therapeutic vaccine comprises a plasmid or a viral vector.

4. The immunostimulatory combination of claim 3 or the first composition for use according to claim 3, wherein said viral vector is obtained from a poxvirus, and preferably a vaccinia virus selected from the group consisting of the Western Reserve, Copenhagen, Wyeth, Lister and MVA strains.

5. The immunostimulatory combination of claim 3 or the first composition for use according to claim 3, wherein said viral vector is an adenovirus, and preferably an adenovirus selected from the group consisting of human, chimpanzee and gorilla adenoviruses and, more specifically, an E1-defective adenovirus.

6. The immunostimulatory combination of any of claims 1 to 5 or the first composition for use according to any of claims 1 to 5, wherein said therapeutic vaccine contains or encodes one or more polypeptide(s) of therapeutic interest, preferably selected from the group consisting of suicide gene products, cytokines and antigens such as cancer antigens or antigens originating from an infectious organism or associated with a disease or a condition caused by an infectious organism.

7. The immunostimulatory combination of claim 6 or the first composition for use according to claim 6, wherein said one or more polypeptide(s) of therapeutic interest is selected from the group consisting of mucin antigens, HPV antigens, Mtb antigens, HBV antigens, the human IL-2, the human GM-CSF and the FCU-1 suicide gene product.

8. The immunostimulatory combination of claim 7 or the first composition for use according to claim 7, wherein said therapeutic vaccine is selected from the group consisting of i) A MVA virus encoding the MUC-1 antigen and human IL-2; ii) A MVA virus encoding membrane anchored HPV-16 non-oncogenic E6 and E7 antigens and human IL-2; iii) A MVA virus encoding the FCU1 gene; vi) A vaccinia virus encoding the FCU1 gene; vii) an Ad virus encoding a fusion of HBV HBc, pol and one or more env immunogenic domain(s) such as a fusion comprising an amino acid sequence having at least 80% identity with SEQ ID NO: 17 or SEQ ID NO: 18 and viii) a MVA virus encoding one or more Mtb antigens.

9. The immunostimulatory combination of any of claims 1 to 8 or the first composition for use according to any of claims 1 to 8, wherein said oligonucleotide comprises from 21 to 60 nucleotides, advantageously from 22 to 50 nucleotides, desirably from 23 to 40 nucleotides, preferably from 24 to 35 nucleotides, more preferably from 25 to 30 nucleotides and even more preferably 26, 27, 28, 29 or 30 nucleotides with an absolute preference for 26 nucleotides.

10. The immunostimulatory combination of claim 9 or the first composition for use according to claim 9, wherein said oligonucleotide has a phosphorothioate backbone.

11. The immunostimulatory combination of claim 9 or 10 or the first composition for use according to claim 9 or 10, wherein said at least RRCGYY (SEQ ID NO:13) hexameric motifs are AACGTT (SEQ ID NO:15) and wherein said RYCGYY (SEQ ID NO:14) hexameric motifs are GTCGTT (SEQ ID NO:16).

12. The immunostimulatory combination of claim 11 or the first composition for use according to claim 11, wherein said oligonucleotide comprises a nucleotide sequence as shown in SEQ ID NO: 10 or a nucleotide sequence as shown in SEQ ID NO: 11.

13. The immunostimulatory combination of any of claims 1 to 12 or the first composition for use according to any of claims 1 to 12, wherein the therapeutic vaccine and the oligonucleotide are formulated for subcutaneous, intramuscular or intratumoral administration route preferably at the same site or at close proximity.

14. The immunostimulatory combination of claim 13 or the first composition for use according to claim 13, wherein said first composition comprises from 10⁴ to 10¹³ pfu or vp of a viral vector and said second composition comprises from 0.25 to 25mg of an oligonucleotide.

15. The immunostimulatory combination of any of claims 1 to 14 or the first composition for use according to any of claims 1 to 14, wherein the first and the second compositions are administered sequentially, with a preference for administration of the first composition being initiated before the administration of the second composition.

16. The immunostimulatory combination of claim 15 or the first composition for use according to claim 15, wherein the time interval between the administration of the first composition and the administration of the second composition varies from approximately 6 hours to approximately 3 days, preferably from approximately 6 hours to approximately 48 hours and more preferably is about 24 hours.

17. The immunostimulatory combination of any of claims 1 to 16 or the first composition for use according to any of claims 1 to 16, for use in the treatment of: (i) a proliferative disease and preferably a proliferative disease selected from the group consisting of renal cancer, bladder cancer, prostate cancer, breast cancer, colorectal cancer, lung cancer, liver cancer, gastric cancer, pancreatic cancer, melanoma, ovarian cancer and glioblastoma, and especially metastatic ones or (ii) an infectious disease resulting from infection with a pathogenic organism selected from the group consisting of bacteria, parasite, virus and fungus and preferably a chronic HBV infection.

18. The immunostimulatory combination of any of claims 1 to 17 or the first composition for use according to any of claims 1 to 17, for use for inducing or enhancing an immune response or function, such as innate immunity.

19. A method of treatment of a proliferative disease or an infectious disease in a subject in need thereof comprising administering to the subject at least (a) a first composition comprising a therapeutic vaccine as described in any of claims 1 to 8 and 13-18 and (b) a second composition comprising one or more oligonucleotide(s) as described in any of claims 1, 2 and 9-18 in an amount sufficient to treat or prevent said proliferative or infectious disease.

20. A method of inducing or stimulating an immune response in a subject in need thereof comprising a) administering to a subject a first composition comprising an immunologically effective amount of a therapeutic vaccine as described in any of claims 1 to 8 and 13-18 and (b) administering to the subject a second composition comprising an immunologically effective amount of one or more oligonucleotide(s) in any of claims 1, 2 and 9-18.

21. The method according to claim 19 or 20, wherein, said a) and b) steps are conducted sequentially with a specific preference for a) being 6-48h before b).

22. The method according to claim 20 or 21, wherein said method provides an induction or a stimulation of an innate immune response.

23. The immunostimulatory combination of claim 18 or the first composition for use according to claim 18 or the method according to claim 22, wherein said induction or enhancement of the innate immune response is preferably correlated with at least one of the following properties:
An increase in the number of macrophages at or at close proximity of the injection site;
An increase in the number of activated CD69+ NK (natural killer) cells at or at close proximity of the injection site;
An increase in the number of KLRG1 (killer cell lectin receptor) positive CD3+ CD8+ lymphocytes at or at close proximity of the injection site;
An increase in the number of activated DC (dendritic cells) in the lymph node draining the injection site;
An increase of the concentration of IL-18 at or at close proximity of the injection site; and/or
An increase of the concentration of IL-1β at or at close proximity of the injection site; and/or
A decrease of CD163 positive cells at or at close proximity of the injection site; or
Any combination of two or more such properties.

24. A method of treatment according to any one of claims 19 or a method according to anyone of claims 20 to 23, wherein said subject is afflicted with a cancer selected from the group consisting of renal cancer, bladder cancer, prostate cancer, breast cancer, colorectal cancer, lung cancer, liver cancer, gastric cancer, pancreatic cancer, melanoma, ovarian cancer and glioblastoma, and especially metastatic ones or with an infectious disease such as a chronic HBV infection.

25. A kit of parts comprising a) the first composition and b) the second composition comprised in the immunostimulatory combination according to any one of claims 1 and 3-18 together with instructions for use.

26. A composition comprising a therapeutically or an immunologically effective amount of an oligonucleotide having at least 21 nucleotides in length and comprising at least three hexameric motifs represented as RRCGYY (SEQ ID NO:13) or RYCGYY (SEQ ID NO:14), wherein each R occurrence is a purine nucleotide or a purine nucleotide derivative; C is a cytosine nucleotide or a cytosine nucleotide derivative; G is a guanosine nucleotide or a guanosine nucleotide derivative; and Y is a pyrimidine nucleotide or a pyrimidine nucleotide derivativePDE5 inhibitor, for use for treating a subject having a chronic infectious disease such as a chronic hepatitis B, with a preference for a composition wherein said oligonucleotide comprises a nucleotide sequence as shown in SEQ ID NO: 10 or a nucleotide sequence as shown in SEQ ID NO: 11.


SEQUENCE LISTING (20 sequences)

SEQ ID NO: 1 - DNA, 6 bp, artificial sequence
  hexameric CpG motif
  misc_feature (2)..(2): n is a, c, g, or t
rncgyy

SEQ ID NO: 2 - DNA, 7 bp, artificial sequence
  heptameric CpG motif
  misc_feature (1)..(1): n is a, c, g, or t
  misc_feature (3)..(3): n is a, c, g, or t
nrncgyy

SEQ ID NO: 3 - DNA, 8 bp, artificial sequence
  octameric CpG motif
  misc_feature (1)..(2): n is a, c, g, or t
  misc_feature (4)..(4): n is a, c, g, or t
nnrncgyy

SEQ ID NO: 4 - DNA, 7 bp, artificial sequence
  heptameric CpG motif
  misc_feature (2)..(2): n is a, c, g, or t
  misc_feature (7)..(7): n is a, c, g, or t
rncgyyn

SEQ ID NO: 5 - DNA, 8 bp, artificial sequence
  octameric CpG motif
  misc_feature (2)..(2): n is a, c, g, or t
  misc_feature (7)..(8): n is a, c, g, or t
rncgyynn

SEQ ID NO: 6 - DNA, 8 bp, artificial sequence
  octameric CpG motif
  misc_feature (1)..(1): n is a, c, g, or t
  misc_feature (3)..(3): n is a, c, g, or t
  misc_feature (8)..(8): n is a, c, g, or t
nrncgyyn

SEQ ID NO: 7 - DNA, 9 bp, artificial sequence
  nonameric CpG motif
  misc_feature (1)..(1): n is a, c, g, or t
  misc_feature (3)..(3): n is a, c, g, or t
  misc_feature (8)..(9): n is a, c, g, or t
nrncgyynn

SEQ ID NO: 8 - DNA, 9 bp, artificial sequence
  nonameric CpG motif
  misc_feature (1)..(2): n is a, c, g, or t
  misc_feature (4)..(4): n is a, c, g, or t
  misc_feature (9)..(9): n is a, c, g, or t
nnrncgyyn

SEQ ID NO: 9 - DNA, 10 bp, artificial sequence
  decameric CpG motif
  misc_feature (1)..(2): n is a, c, g, or t
  misc_feature (4)..(4): n is a, c, g, or t
  misc_feature (9)..(10): n is a, c, g, or t
nnrncgyynn

SEQ ID NO: 10 - DNA, 26 bp, artificial sequence
  sequence of  litenimod (Li28)
taaacgttataacgttatgacgtcat

SEQ ID NO: 11 - DNA, 24 bp, artificial sequence
  CpG oligonucleotide ODN2006
tcgtcgttttgtcgttttgtcgtt

SEQ ID NO: 12 - protein, 557 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPSPGSTAPPAHGVTSAPDTRPAPGSTAPPAHGVTSAPDTRPAPGSTAPPAHGVTSAPDTRPAPGSTAPPAHGVTSAPDTRPAPGSTAPPGHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 13 - DNA, 6 bp, artificial sequence
  hexameric CpG motif
rrcgyy

SEQ ID NO: 14 - DNA, 6 bp, artificial sequence
  hexameric CpG motif
rycgyy

SEQ ID NO: 15 - DNA, 6 bp, artificial sequence
  hexameric CpG motif
aacgtt

SEQ ID NO: 16 - DNA, 6 bp, artificial sequence
  hexameric CpG motif
gtcgtt

SEQ ID NO: 17 - protein, 1007 aa, Artificial Sequence
  fusion between truncated core (1-148)- mutated      Y07587 polymerase - env1 and env2
MDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRLGLQSQQGHLARRQQGRSWSIRAGIHPTARRSFGVEPSGSGHSTNLASKSASCLYQSPVRKAAYPAVSTFEKHSSSGHAVELHNLPPNSARSQSERPVFPCWWLQFRNSKPCSDYCLSHIVNLLEDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHLYSHPIILGFRKIPMGVGLSPFLLAQFTSAICSVVRRAFPHCLAFSVLQAGFFLLTRILTIPQSLDSWWTSLNFLGGTTVCLGQGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSKQAFTFSPTYKAFLCKQYLNLYPVARQRPGLCQVFAHATPTGWGLVMGHQRMRGTFLWASARFSWLSLLVPFVQWFVGLSPTVWLSVSRKYTSFPWLLGCAANWILRGTSFVYYPSALNPYHDPSRGRLGLSRPLLRLPFRPTTGRTSLYADSPSVPSHLPDRVHFASPLHVAWRPP

SEQ ID NO: 18 - protein, 1095 aa, Artificial Sequence
  fusion between signal sequence -core (1′148)-      mutated Y07587 polymerase -env1-env2 and TM sequence
MVPQALLFVPLLVFPLCFGKFPIDIDPYKEFGATVELLSFLPSDFFPSVRDLLDTASALYREALESPEHCSPHHTALRQAILCWGELMTLATWVGGNLEDPISRDLVVSYVNTNMGLKFRQLLWFHISCLTFGRETVIEYLVSFGVWIRTPPAYRPPNAPILSTLPETTVPLSYQHFRRLLLLDDEAGPLEEELPRLADEGLNRRVAEDLNLGNLNVSIPWTHKVGNFTGLYSSTVPVFNPHWKTPSFPNIHLHQDIIKKCEQFVGPLTVNEKRRLQLIMPARFYPNVTKYLPLDKGIKPYYPEHLVNHYFQTRHYLHTLWKAGILYKRETTHSASFCGSPYSWEQKLQHGAESFHQQSSGILSRPPVGSSLQSKHRKSRLGLQSQQGHLARRQQGRSWSIRAGIHPTARRSFGVEPSGSGHSTNLASKSASCLYQSPVRKAAYPAVSTFEKHSSSGHAVELHNLPPNSARSQSERPVFPCWWLQFRNSKPCSDYCLSHIVNLLEDWGPCAEHGEHHIRIPRTPARVTGGVFLVDKNPHNTAESRLVVDFSQFSRGNYRVSWPKFAVPNLQSLTNLLSSNLSWLSLDVSAAFYHLPLHPAAMPHLLVGSSGLSRYVARLSSNSRIFNYQHGTMQNLHDSCSRNLYVSLLLLYQTFGRKLHLYSHPIILGFRKIPMGVGLSPFLLAQFTSAICSVVRRAFPHCLAFSVLQAGFFLLTRILTIPQSLDSWWTSLNFLGGTTVCLGQGAKSVQHLESLFTAVTNFLLSLGIHLNPNKTKRWGYSLHFMGYVIGCYGSLPQDHIIQKIKECFRKLPVNRPIDWKVCQRIVGLLGFAAPFTQCGYPALMPLYACIQSKQAFTFSPTYKAFLCKQYLNLYPVARQRPGLCQVFAHATPTGWGLVMGHQRMRGTFLWASARFSWLSLLVPFVQWFVGLSPTVWLSVSRKYTSFPWLLGCAANWILRGTSFVYYPSALNPYHDPSRGRLGLSRPLLRLPFRPTTGRTSLYADSPSVPSHLPDRVHFASPLHVAWRPPYVLLSAGALTALMLIIFLMTCCRRVNRSEPTQHNLRGTGREVSVTPQSGKIISSWESHKSGGETRL

SEQ ID NO: 19 - protein, 10 aa, Artificial Sequence
  HBV Pol-specific VSA peptide
VSAAFYHLPL

SEQ ID NO: 20 - protein, 9 aa, Artificial Sequence
  Ad-specific FAL peptide
FALSNAEDL